(19) United States
(12) Patent Application Publication
(10) Pub. No.: US 2026/0104424 A1
(43) Pub. Date: Apr. 16, 2026

(54) METHODS, SYSTEMS AND KITS FOR IDENTIFYING BIOACTIVE COMPOUNDS AND THERAPEUTIC METHODS AND COMPOSITIONS

(71) Applicants: The Johns Hopkins University, Baltimore, MD (US); SEOUL NATIONAL UNIVERSITY R&DB FOUNDATION, Seoul (KR)

(72) Inventors: Harry Larman, Baltimore, MD (US); Meng-Hsuan Hsiao, Baltimore, MD (US); Zixing Liu, Baltimore, MD (US); Martin Steinegger, Gwanak-gu (KR)

(21) Appl. No.: 19/101,854

(22) PCT Filed: Aug. 7, 2023
(86) PCT No.: PCT/US2023/029669

§ 371 Date: Feb. 6, 2025

Related U.S. Application Data
(60) Provisional application No. 63/395,814, filed on Aug. 6, 2022.

(51) Int. Cl. G01N 33/68; C12N 15/10

(52) U.S. Cl. CPC G01N 33/6845; C12N 15/1037; C12N 15/1058; C12N 15/1089

(57) ABSTRACT

An extracellular vesicle (EV) associated with a viral vector is provided. The combination of the EV and virus vectors provide widespread and highly efficient transgene expression in lungs, following localized administration, as well as in mucus-covered air-liquid interface (ALI) cultures with primary human bronchial epithelial (HBE) cells and nasal epithelial (HNE) cells.

### CROSS-REFERENCE TO RELATED APPLICATION

The present application claims priority of U.S. provisional application No. 63/395,814 filed Aug. 6, 2022, which is incorporated by reference herein in its entirety.

### STATEMENT REGARDING FEDERALLY SPONSORED RESEARCH

This invention was made with government support under grant no. R01GM136724, awarded by the National Institutes of Health. The government has certain rights in the invention.

### FIELD

In one aspect, the present disclosure provides systems and methods for identifying or detecting a bioactive peptide. Such systems and methods include contacting at least one target with a sample comprising a library of molecularly displayed peptides and determining whether a peptide in the sample selectively binds to the at least one target, thereby identifying or detecting candidate bioactive peptides.

In a further aspect, treatment methods and pharmaceutical compositions are provided.

### BACKGROUND

Stable polypeptide structures, particularly those incorporating disulfide bonds, have been evolutionarily co-opted for myriad biological functions that range from signaling, to defense, to predation.¹⁻⁵

It would be desirable to identify additional stable compounds for therapeutic and other applications.

### SUMMARY

We now provide new methods and systems for identifying candidate bioactive compounds, including peptide-based molecule that have therapeutic use.

More particularly, in one aspect, a method of identifying candidate bioactive compounds is provided, the method comprising: (a) preparing and cloning an oligonucleotide library encoding diverse toxin-like polypeptides from a multitude of scaffolds to create a polypeptide display library; (b) contacting the toxin-like polypeptide display library with target molecules; (c) removing members of the toxin-like polypeptide display library that do not sufficiently bind to the target molecules; (d) sequencing the members of the toxin-like polypeptide display library which bind to the target; and (e) identifying candidate bioactive compounds.

In certain aspects, the polypeptide display library is comprised of one or more of a mRNA display library, a ribosome display library, a PLATO library, a MIPSA library, a PepSeq library, a bacteriophage display library, a hyperphage display library, a bacterial display library, a yeast display library, an insect cell display library, an invertebrate cell display library or a mammalian cell display library.

In certain preferred aspects, a polyvalent display display is utilized. In certain preferred aspects, a bacteriophage display or hyperphage display is utilized. In certain aspects, the polyvalent display, bacteriophage display or hyperphage display comprises M13 polyvalent display, M13 bacteriophage display or M13 hyperphage display.

In certain aspects, the toxin-like polypeptide may be covalently linked (fused) to the N-terminus of the p3 protein.

In certain aspects, the toxin-like polypeptide display library includes sequences identified from metagenomic database similarity searching using toxin or toxin-like sequences or structures.

In certain aspect, a toxin-like polypeptide display library may include sequences generated from a computer program to resemble toxin-like polypeptides. In certain embodiments, the computer program may involve an artificial intelligence or machine learning approach. In certain embodiments, the computer program may involve structure-guided sequence design.

In certain aspects the displayed toxin-like polypeptides are designed to be their mature forms.

In certain aspects the displayed toxin-like polypeptides are designed to be their active forms.

In certain aspects, the displayed toxin-like polypeptides are designed to contain between 0 and 5, 10, 15, 20, 25 or 30 or more cysteine residues one or more of which are capable of forming cysteine bonds. In certain aspects, the displayed toxin-like polypeptides suitably contain at least 1 or 2 cysteine residues, preferably which are capable of forming disulfide bonds.

In certain aspects, the displayed toxin-like polypeptides are fused with a polypeptide linker, for example to promote their availability to bind a target.

In certain aspects, the oligonucleotide library encoding diverse toxin-like polypeptides contains degenerate nucleotide positions.

In certain aspects, the oligonucleotide library encoding diverse toxin-like polypeptides is mutagenized or undergoes error prone PCR in order to introduce library diversity.

In certain aspects, the number of rounds of target binding is one. In certain aspects, the number of rounds of target binding is greater than one.

In certain aspects, the toxin-like polypeptide display library is mutagenized between rounds of target binding.

In certain aspects, the toxin-like polypeptide display library is sequenced after and/or between rounds of target binding.

In certain aspects, the target molecules are purified and conjugated to a surface during or after interaction with the toxin-like polypeptide display library.

In certain aspects, the target molecules are expressed in or on target cells.

In certain aspects, the library is sequenced using high throughput DNA sequencing.

In certain aspects, sequencing of the library is used to identify differential binding of toxin-like polypeptide library members to target molecules versus molecules or surfaces lacking the target or to different molecules that are related but not identical to the target molecules.

In certain aspects, the oligonucleotide library encoding diverse toxin-like polypeptides is comprised of shuffled domains.

In certain aspects, the displayed toxin-like polypeptides are designed to also display library members that lack individual or multiple cysteine residues to identify disulfide binds that are critical for binding to the target molecules.

In certain aspects, the displayed toxin-like polypeptides comprise versions of candidate bioactive compounds with targeted mutations, such as scanning mutagenesis, or tiled fragments, for the purpose of identifying key residues or regions that are critical for binding to the target molecules.

In certain aspects, residues or regions that facilitate binding to the target molecules are further optimized for improved binding.

In certain aspects, the candidate bioactive polypeptides identified by sequencing are selected for evaluation by one or more assays and/or statistical evaluation.

In another embodiment, a method of identifying candidate therapeutic compounds, the method comprising: (a) preparing and cloning an oligonucleotide library encoding candidate therapeutic polypeptides into a hyperphage phagemid and culturing in bacteria; (b) infecting the bacteria with a helper phage lacking a native P3 protein and generating a phage library; (c) contacting the phage library with ligands or cells expressing a target protein; (d) sequencing of the phage library bound to the ligands or target proteins; and identifying candidate therapeutic compounds.

In certain aspects of the present methods, the candidate therapeutic polypeptides comprise diverse folding patterns.

In certain aspects of the present methods, the candidate therapeutic polypeptides comprise diverse disulfide bonding patterns.

In certain aspects of the present methods, the candidate therapeutic polypeptides comprise peptides isolated from toxins or venoms.

In certain aspects of the present methods, the candidate therapeutic polypeptides displayed on phage surfaces further comprise a linker linking the candidate therapeutic polypeptides to phage surface coat proteins.

In certain embodiments, candidate therapeutic polypeptides identified by sequencing are selected for evaluation by one or more assays and/or statistical evaluation.

Additionally, in certain aspects, identified candidate therapeutic polypeptides are subjected to a second-stage screening comprising comparing identified sequences to genomic databases, computer-based structural modeling or machine learning executable programs.

In certain embodiments of the present methods, candidate therapeutic polypeptides identified in either screening stage are subjected to random mutagenesis or insertion of degenerate sequences.

In further embodiments, kits are provided, including kits for identifying candidate bioactive compounds comprising a diverse toxin-like polypeptide display library comprised of multitude scaffolds.

In additional embodiments, kits are provided identifying candidate bioactive compounds that comprise a library of DNA encoding diverse toxin-like polypeptides from a multitude of scaffolds to be used for creating a polypeptide display library.

In yet additional embodiments, kits are provided that comprise materials for conducting a method as disclosed herein.

A diverse sequence as described herein is a sequence which varies between the members of a population i.e. the sequence is different in different members of the population. A diverse sequence may be random i.e. the identity of the amino acid or nucleotide at each position in the diverse sequence may be randomly selected from the complete set of naturally occurring amino acids or nucleotides or a sub-set thereof.

Preferably, a library is a display library. The binding members in the library may be displayed on the surface of particles, or molecular complexes such as beads, ribosomes, cells or viruses, including replicable genetic packages, such as yeast, bacteria or bacteriophage (e.g. Fd, M13 or T7) particles, viruses, cells, including mammalian cells, or covalent such as mRNA display or MIPSA, ribosomal or other in vitro display systems. Each particle or molecular complex may comprise nucleic acid that encodes the binding member that is displayed by the particle and optionally also a displayed partner domain if present. In other cases, the molecular complex may comprise nucleic acid that uniquely identifies the displayed polypeptide.

In a further aspect, methods for treating a mas-related G protein coupled receptor-mediated (MrgprX4) condition in a subject are provided and suitably comprise administering an effective amount of a compound comprising a Kunitz-type domain to the subject, thereby treating the G protein coupled receptor-mediated condition. In particular aspects, the mas-related G protein coupled receptor-mediated condition may be adverse drug reactions (e.g., Stevens-Johnson Syndrome (SJS)), pruritus including cholestatic pruritus and other chronic itch conditions, and autoimmune diseases (e.g. multiple sclerosis).

In a particular aspect, methods for treating a chronic itch condition or disorder in a subject are provided and suitably comprise administering an effective amount of a compound comprising a Kunitz-type domain to the subject, thereby treating the chronic itch condition or disorder.

Suitably, a subject may be identified as suffering from or susceptible to a mas-related G protein coupled receptor-mediated condition such as an adverse drug reaction (e.g., Stevens-Johnson Syndrome (SJS)), pruritus including cholestatic pruritus and other chronic itch conditions, and/or an autoimmune disease (e.g. multiple sclerosis), and the compound comprising a Kunitz-type domain is administered to the identified subject. Preferably, the subject is a human.

The administered compound suitably may be a Kunitz-type protease inhibitor class protein that comprises a Kunitz-type inhibitor domain, which can be characterized by six cysteines forming a distinctive disulfide bond pattern of C1-C6, C2-C5, C3-C4. Specific Kunitz-type compounds that may be administered in accordance with these present therapeutic methods include TFPI (including human TPPI) and ERR1712142 and derivatives thereof. FIG. 21 also provides additional Kunitz-type compounds that may be administered in accordance with these present therapeutic methods. Additional preferred Kunitz-type compounds are reported or are commercially available and can be identified empirically. Preferred compounds for use in the present methods and pharmaceutical compositions include TPPI/Kunitz inhibitor 1, TPPI/Kunitz inhibitor 2, and TPPI/Kunitz inhibitor 3 (sequences set forth in FIGS. 29B and 31) and derivatives of each of those. Additional preferred compounds for use in the present methods and pharmaceutical compositions include the compounds set forth in FIG. 32A-32D and derivatives of those compounds.

### Definitions

It is understood that the present disclosure is not limited to the particular methods and components, etc., described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to a 5 “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this disclosure belongs. Specific methods, devices, and materials are described, although any methods and materials similar or equivalent to those described herein can be used in the practice or testing of the present disclosure.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present disclosure.

### Definitions

By “fragment” is meant a portion of a nucleic acid molecule or polypeptide. This portion contains, or at least 1%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, 100, 200, 300, 400, 500, 600, 700, 800, 900, or 1000 nucleotides or amino acids.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a pathogen.

By “reference sequence” is meant a defined sequence used as a basis for sequence comparison. A reference sequence may be a subset of or the entirety of a specified sequence; for example, a segment of a full-length cDNA, RNA, or gene sequence, or the complete cDNA, RNA, or gene sequence. For polypeptides, the length of the reference polypeptide sequence will generally be at least about 16 amino acids, or at least about 20 amino acids, more or at least about 25 amino acids, and even more or about 35 amino acids, about 50 amino acids, or about 100 amino acids. For nucleic acids, the length of the reference nucleic acid sequence will generally be at least about 40 nucleotides, or at least about 60 nucleotides, more or at least about 75 nucleotides, or about 100 nucleotides or about 300 nucleotides or any integer thereabout or there between.

“Percentage of sequence identity” or similar term of sequence identity is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A derivatives of a peptide or compound as referred to herein suitably may include a peptide whose partial or full amino acid sequence shows approximately at least 50%, 50-60%, 60-70%, 70-80%, 80%, 90%, 95%, 97%, 98%, 99% or 99.8% identity to a reference sequence. Preferably, such a derivative also may be a functional derivative and demonstrate efficacy or activity in an in vitro or in vivo assay of a present disease or disorder disclosed herein such as a chronic itch condition.

By “sensitivity” is meant a percentage of subjects correctly identified as having a particular disease or condition, or pathogen.

By “specificity” is meant a percentage of subjects correctly identified as NOT having a particular disease or condition, or pathogen, i.e., normal or healthy subjects.

By “subject” is meant any individual or patient to which the method described herein is applied. Generally, the subject is human, although as will be appreciated by those in the art, the subject may be an animal (e.g., pet, agricultural animal, wild animal, etc.), disease vector (e.g., mosquitoes, sandflies, triatomine bugs, blackflies, ticks, tsetse flies, mites, snails, lice, etc.), or an environmental sample (e.g., sewage, food products, etc.). Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “library” refers to a collection of members. A library may be comprised of any type of members. For example, in some embodiments, a library comprises a collection of phage particles. In some embodiments, a library comprises a collection of peptides. In some embodiments, a library comprises a collection of cells. A library typically includes diverse members (i.e., members of a library differ from each other by virtue of variability in an element, such as a peptide sequence, between members). For example, a library of phage particles can include phage particles that express unique peptides. A library of peptides can include peptides having diverse sequences. A library can include, for example, at least 10¹, 10², 10³, 10⁴, 10⁵, 10⁶, 10⁷, 10⁸, 10⁹, or more unique members.

The term “ligand” refers to any agent that binds to a receptor. Ligands can include, but are not limited to, small molecules (whether synthetic or isolated from natural sources), biodegradable cofactors, proteins, synthetic peptides, and polymers, both synthetic and naturally occurring, including DNA. In many embodiments, ligands are polypeptides. For example, ligands can be protein/peptide toxins and/or other venom/poison components of animal, plant, or microbial origin or natural or synthetic derivatives of the same. In some embodiments, a ligand is a toxin peptide as defined herein. In some embodiments, ligands are expressed and/or presented to a receptor as part of a library, e.g., a phage display library. In some embodiments, ligands are expressed and/or presented singly. A ligand can be presented to a receptor in any other mean or form (e.g., removed from the phage, or expressed in a comparable/other expression systems) suitable for ligand-target selection and/or ligand validation. In some embodiments, a ligand is a phage-only peptide to monitor or alter a selection process. According to methods described herein, ligands can be selected on any of a variety of bases, including for example on the basis of a particular affinity, specificity, or activity toward a receptor of interest. In certain embodiments, a ligand binds to a receptor with a K_{D} of 1×10⁻⁶ M or less, 1×10⁻⁷ M or less, 1×10⁻⁸ M or less, 1×10⁻⁹ M or less, 1×10⁻¹⁰ M or less, 1×10⁻¹¹ M or less, or 1×10⁻¹² M or less. In certain embodiments, a ligand binds a receptor which is a channel, and inhibits an activity of the channel (e.g., ion transport) with an IC₅₀ of 1×10⁻⁶ M or less, 1×10⁻⁷ M or less, 1×10⁻⁸ M or less, 1×10⁻⁹ M or less, 1×10⁻¹⁰ M or less, 1×10⁻¹¹ M or less, or 1×10⁻¹² M or less. In some embodiments, a ligand has specificity a particular receptor such that the ligand binds to the receptor and/or modulates an activity of the receptor with an affinity/potency that is at least twice, 4 times, 5 times, 10 times, 100 times, 1000 times as great as for another receptor in the same class. To give one example, in some embodiments, a ligand binds to one type of potassium channel, Kv 1.3, with an affinity that is at least 10 or 100 times greater than its affinity for another potassium channel (e.g., KvI.1 or KvI.2).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 (includes FIGS. 1A-1G) shows an overview of the candidate bioactive peptide discovery platform.

FIG. 2 shows an initial diverse phage library generation.

FIG. 3 shows steps of candidate verification and prioritization.

FIG. 4 shows candidate library diversification.

FIG. 5 shows composition of an example animal toxin library. In the depicted circular graph, gastropod is at 44%; arachnid is at 33%; reptile is at 14%.

FIG. 6 shows animal toxin phage library and expanded diversity library.

FIG. 7 (includes FIGS. 7A and 7B) shows candidate ligands discovered for Epidermal Growth Factor Receptor (EGFR).

FIG. 8 (includes FIGS. 8A-8G: M13 polyvalent phage display workflow identifies ligands to EGFR and C—X—C chemokine receptor 2 (CXCR2). A. (i) An oligonucleotide library pool encoding full-length 90aa polypeptides is designed and synthesized for cloning into the engineered M13 phagemid vector. (ii) Oligonucleotide library is cloned into the M13 hyperphage display system and phage are grown in E. coli bacteria cells. Proteins of interest fused with the M13 P3 protein are displayed in 5 copies on the phage surface. (iii) The phage library is screened against a target for affinity purification: bead-based screening (left) or cell-based screening (right) (iv-v). (vi) The DNA of the enriched phage library is collected and amplified by PCR for sequencing (vii). (viii) Informatic analysis identifies candidate binder peptides. B. The effect of linker length and receptor expression level on EGF binding to EGFR. C. Evaluation of disulfide bond formation and its impact on EGF binding to EGFR. D. The human secretome library, categorized by molecular function. ECM, extracellular matrix. IgSF, immunoglobulin superfamily. E, F. Volcano plot depicting the ligand enrichment results of phage library screening against cells overexpressing EGFR (E) or CXCR2 (F). Each data point in the plots represents a unique peptide from the phage library. Enriched known or endogenous ligands are highlighted with distinct colors. The spiked-in control is depicted in black, and proteins not enriched from the screening are represented as grey dots. Methods for hits determination can be referred to in the method section. G. Anti-pERK1/2 western blot image demonstrating EGFR activation by recombinantly expressed protein: MPXV, EGF-like protein form monkeypox virus. Mg1a, ant venom.

FIG. 9 (includes FIGS. 9A-9E) Animal venom library design and characterization. A. 21,311 parent animal venom protein sequences were obtained from Uniprot. Of these, 11,128 sequences possessing annotations for mature and active forms were extracted. Oligonucleotides encoding unique protein sequences equal to or less than 90 amino acids in length, regardless of their mature and active form status, were subsequently used to create the M13 animal venom phage library. B. Length distribution for AT sequences before and after the extraction of their mature and active forms. The median sequence length for the parent dataset is 110-aa, which reduces to 62-aa in their mature and active forms. C. The inner layer represents the animal phylum while the outer layer depicts the animal class. Refer to Table S2 for more detailed information. D. Distribution of the number of cysteines in the AT library. E. Evaluation of the ratio of number of disulfide bonds on the AT phage library in comparison to the phagemid library stock (Y-axis), versus number of disulfide bonds expected based on protein annotations from Uniprot (X-axis). The phage library was IPed with anti-FLAG antibody. The number of disulfide bonds are shown according to information from Uniprot, where ‘NA’ indicates sequences that possess cysteines but have unknown disulfide bond patterns. The red dashed line marks the median ratio (0.59) for the phage library. A two-tailed, unpaired T-test with Welch's correction was used to assess the statistical significance of the decrease in ratio for library members with four or more disulfide bonds. A p-value of less than 0.05 was considered significant and is denoted by a single asterisk.

FIG. 10 (includes FIGS. 10A-10D) FIG. 3. Metavenome library design and characterization. A. (i) Animal venoms were used as annotated queries to search for homologous sequences in two databases, the Big Fantastic Database (BFD) and Serratus. (ii) Identical target sequences and those exceeding 100 amino acids were removed. (iii) To create a diverse yet compact sequence set for the library, metagenomic sequences were clustered and excluded if they showed at least 50% sequence identity and a 95% overlap with an animal venom sequence. (iv) The final metavenome library consisted of sequences of 90 amino acids or shorter, resulting in 42,136 unique sequences that were synthesized as the metavenome library DNA pool. These sequences were subsequently cloned and packaged to form the metavenome phage library. B. The distribution of the number of metavenome sequences that corresponded to each animal venom. C. Heat map depicting the correlation of the number of cysteines between animal venom and metavenome sequence pairs. D. Composition of the metavenome library based on animal classification. The inner layer represents the animal phylum while the outer layer depicts the animal class. See Table S5 for more detailed information.

FIG. 11 (includes FIGS. 11A-11E). Discovery of Diverse Scaffolds and Ligands Targeting EGFR from the Animal Venom, Metavenome and Secretome Libraries. A. Volcano plot analysis of AT, MV and secretome phage libraries bead-based screening of the EGFR-Ig chimera. Each point represents a unique library member, with significantly enriched clones highlighted in yellow. Endogenous and known ligands are color-coded as in FIG. 1E. B. Sequence-based network graph depicting sequence similarities among candidates. Dot color corresponds to the originating library. C. MSA analysis illustrates conserved amino acids shared among sequences within their respective clusters. Black underlines indicate the disulfide bond patterns annotated in the Uniprot database. D. The fold-change values represent ligands binding to EGFR in the presence and absence of EGF in clusters 1-3. The statistical significance of the fold-change values with and without EGF was assessed using a paired T-test. The mean differences of the fold-change values within the paired dots are displayed beneath each cluster. E. Binding modes of selected ligands in each cluster predicted using RosettaDock.

FIG. 12 (includes FIGS. 12A-12E). Kunitz type inhibitor is a potential antagonist for MrgprX4 discovered from the metavenome library. A. Volcano plot analysis of animal venom, metavenome and secretome phage libraries screening against HEK293 cells overexpressing MrgprX4. Each point represents a unique peptide in the AT, MV and secretome libraries. B. Heat map showing selectivity of fold-change values of the 6 peptide candidates across the HEK293-MrgX families. C. MSA analysis and motif discovery with MEME Suite identifies conserved amino acids shared among the candidate hits (left). Structural alignment of the hits using AlphaFold (right). D Flow cytometry analysis of ERR1712142 105-166 binding to HEK293-MrgprX4 and HEK293 cells. MFI, mean fluorescence intensity. E. Calcium signaling assay demonstrates that TFPI is inhibiting the activation of UDCA (EC80), the endogenous ligand of MrgprX4, at 1 and 0.3 uM (n=7) (left panel). For comparison, a negative control protein, osteoprotegerin (OPG), produced in the same manner as the TFPI, was used to assess the inhibitory activity at 1 uM (n=4) (right panel). Peak heights represent the fluorescent intensities normalized against the baseline fluorescent intensities (t=0s). The values displayed on the histogram correspond to the average differences between the maximum normalized peak heights and the normalized peak heights prior to the addition of UDCA (t=25s). A two-tailed, unpaired T-test with Welch's correction was used to assess the statistical significance of the inhibition activity of TFPI at different concentrations.

FIG. 13 shows composition of the human secretome library FIG. 14 shows sequences of tested M13 linker designs.

FIG. 15 shows an animal venom library composition.

FIG. 16 shows the animal venom library quality analysis.

FIG. 17 shows the metavenome library quality analysis.

FIG. 18 shows the metavenome library composition: top 10 phyla.

FIG. 19 show a contact map of ERR1712142|105-166-MrgprX4 docking results from RosettaDock.

FIG. 20 shows the contact map of ERR1712142|105-166-MrgprX4 docking results from AlphaFold.

FIG. 21 shows additional compounds related to ERR1712142|105-166 in human proteins identified with Foldseek.

FIG. 22 (includes FIGS. 22A-22D). M13 phagemid design and linker selection. A. Depiction of the M13 phagemid vector with the key components labelled: POI, peptide of interest (orange), Flag tag (green), G4S linker (grey), PAS linkers (blue), P3 protein (purple), and restriction digestion sites (black lines). The linker lengths range from 13 (NL) to 291 amino acids. B. Flow cytometry analysis of EGFR expression levels on four cell lines: CHOK1-PDL1 (negative control cell line), NCI-H358 (low), MDA-MB-231 (medium), and MDA-MB-468 (high). The lower expression level peaks represent background staining with only the fluorescent anti-mouse mAb, and the higher expression level peaks represent cells stained with anti-EGFR mAb (primary mAb) and detected with a fluorescent anti-mouse mAb. C. Flow cytometry analysis of CXCR2 expression levels on HEK293T cells (CXCR2−) and HEK293T-CXCR2 (CXCR2+) cells. The lower expression level peaks represent background staining with only the fluorescent anti-mouse mAb, and the higher expression level peaks represent cells stained with anti-CXCR2 mAb (primary mAb) and detected with a fluorescent anti-mouse mAb. D. Graph depicting the impact of linker length on the fold change value of IL8 binding to CXCR2. $-Lactoglobulin ($-LG), serving as a negative control protein, exhibits low non-specific binding to HEK293T-CXCR2 cells.

FIG. 23 shows composition of the human secretome library based on molecular functions. The histogram depicts the distribution of annotated protein molecular function versus amino acid length.

FIG. 24 (includes FIGS. 24A-24E). Protein production, purification, and quantification. A. Schematic of the engineered pLicC-MBP vector featuring a 6×His tag positioned at the N-terminal side of MBP, followed by a FLAG tag. The peptide of interest (POI) is cloned immediately downstream of an enterokinase (EK) cleavage site, ensuring a clean C-terminus following enzymatic digestion. B. A step-by-step depiction of the MBP fusion protein purification procedure utilizing nickel resins. Detailed descriptions of each step are provided in the methods section. C and D. Coomassie-stained gels highlighting His-MBP-U18-MYRTX-Mril a (C) and His-MBP-Pox (D) before and after EK digestion. The first lane in each panel shows the purified MBP fusion protein without EK cleavage, serving as a control, while the second lane demonstrates the result of protein post-EK digestion, highlighting the complete cleavage of the fusion protein. E. Protein quantification with Coomassie-stained gel. A serial dilution of both a known protein ladder and the POI were run on an analytical gel, followed by quantifying the bands' intensity to determine protein concentration. The specific approach and steps of this densitometry-based image analysis for protein quantification are further detailed in the Methods.

FIG. 25 (includes FIGS. 25A-25C). Metavenome library characterization. A. The abundance of each type of amino acid in the peptide sequences from both animal venom and metavenome libraries. Each set of bars represent a unique amino acid, with their heights corresponding to their relative abundance in the analyzed peptide sequences. Heat map depicting the correlation of the number of cysteines between animal venom and metavenom sequence pairs. IN FIG. 25A, in each pair of bars, AT is positioned on the left and Meta is positioned on the right. B. Analysis of amino acid abundance and correlation between animal venom and metavenom peptide sequence pairs. The Spearman correlation coefficient calculated from protein sequence pairs, with each bar corresponding to a distinct amino acid. The correlation coefficient is derived based on the quantity of each amino acid present in the animal venom and metavenom protein sequence pairs. C. Assessment of the impact of the cysteine count on the metavenome phage library in comparison to its phagemid library stock, per number of expected cysteines. The same experimental method described for FIG. 2E was used.

FIG. 26 (includes FIGS. 26A-26D). Animal venom and metavenome library quality analysis. A. Animal venom phagemid library stock QC: 99.58% of library members were detected; 98.2% of the library was within one log of the mean (indicated by vertical dashed lines). B. Animal venom phage library QC: 90.78% of library members were detected; 87.8% of the library was within one log of the mean. C. Metavenome phagemid library stock QC: 98.1% of library members were detected; 94.9% of the library was within one log of the mean. D. Metavenome phage library stock QC: 94.98% of library members were detected; 88.2% of the library was within one log of the mean.

FIG. 27 (includes FIGS. 27A-27E). Metavenome library characterization. A. Library members annotated from NCBI-BLAST+. B-E. Essential parameters from NCBI-BLAST+search outcomes with black dotted line in each plot highlighting the median values.

FIG. 28. Enrichment of EGFR ligands in cluster 1-3 from the animal venom and metavenome libraries. Fold change values for ligands binding to EGFR across clusters 1-3 are displayed in each heatmap cell. The left column presents screening results in the absence of EGF, while the right column depicts those obtained in the presence of EGF.

FIG. 29. Structural Homologs to ERR1712142|105-166 in human proteins identified with Foldseek. A. MSA analysis illustrates conserved amino acids shared between the 20 homologs and ERR17121421105-166. Black underlines represent the disulfide bond patterns as annotated in the Uniprot database. B. The TFPI protein, encompassing three Kunitz-type domains, exhibits a high-confidence structural alignment with ERR1712142|105-166.

FIG. 30. ERR1712142|105-166 docking result. A. Contact map of ERR1712142|105-166-MrgprX4 docking results via RosettaDock and AlphaFold. Amino acids between ERR1712142|105-166 and MrgprX4 within a 5 A radius are colored; consensus predictions between both modeling methods are indicated in red, while unique RosettaDock and AlphaFold predictions are displayed in grey and blue, respectively. B. Visualization of the docking prediction from RosettaDock, with consensus amino acid interactions highlighted in red.

FIG. 31 shows sequences of TPPI/Kunitz inhibitor 1, TPPI/Kunitz inhibitor 2 and TPPI/Kunitz inhibitor 3.

FIG. 32 (includes FIGS. 32A-32D) shows agents useful in the present methods and compositions. The listed target sequence (Target seq.) is the sequence of the agent.

### DETAILED DESCRIPTION

Animal venoms have emerged as a vast reservoir of bioactive molecules with unexplored potential for drug discovery. These naturally occurring toxins, with their high potency, selectivity, and unique structural features, have evolved over millions of years to target various biological systems¹⁻⁴. A key characteristic of many animal venoms is the presence of multiple disulfide bridges, which lock the polypeptide chain into a single conformation, resulting in distinct properties that contribute to their potent bioactivities, including resistance to protease digestion and high binding selectivity and affinity^{4,5}.

Recent, well-known successes of toxin-derived therapeutics include ziconotide^{4,6}, an analgesic derived from cone snail venom, as well as exenatide^{4,6} and tirzepatide^{7,8}, antidiabetic drugs derived from Gila monster venom. In addition, venom-derived drugs have been successfully developed for a variety of clinical applications, including anticoagulation and chemotherapy, underscoring the broad versatility of these natural compounds^{6,9}.

Membrane proteins play vital roles in numerous cellular processes and are targeted by approximately 30-40% of FDA approved therapeutics”. However, the technical complexity of screening against membrane proteins and assaying their function necessitate innovative drug discovery strategies^{2,13}. One such strategy involves utilizing miniproteins, small proteins typically consisting of 20-100 amino acids, as scaffolds for drug development¹⁴⁻⁶. Compounds derived from these scaffolds, such as designed ankyrin repeat proteins (DARPins) and affibodies¹⁶⁻¹⁷, may then be optimized for specific therapeutic indications. Due to their compact size, stability, potency, and ease of engineering, peptides and miniproteins can provide especially attractive scaffolds for deriving novel membrane protein targeting therapeutics^{16,17}.

Cysteine-reinforced miniproteins utilize disulfide bonds to facilitate proper folding, impart structural rigidity and reduce protease susceptibility. A variety of animal venoms are known as cysteine dense proteins (CDPs), which are frequently found in spider and snake venom¹⁶. Kunitz type domains share similarities with CDPs but are ubiquitously found in natural proteins, feature a hydrophobic core, and have evolved for protease resistance^{16,18,19}. These cysteine-rich miniproteins occur in proteins from exceptionally diversity organisms, especially from animal venoms, representing an abundance of structurally varied scaffolds that share similar biophysical properties^{16,20} In pursuit of a high-throughput approach to discover novel drug candidates from diverse venom and CDP scaffolds, we extracted all full-length, active (mature) venoms and poisons contained in the Uniprot database^{21,22}, which range in size from 2 to 90 amino acids long (−0.2 to 10 kDa) and can thus be encoded by commercially available synthetic oligonucleotide libraries. Of the mature venom sequences in Uniprot, peptides below 90 aa and can thus be represented in such a library. To expand the structural diversity of this venom scaffold library, we performed sequence homology searching of the Big Fantastic Database (BFD)²³ and Serratus databases²⁴, which contain over 2.5 billion metagenomic DNA sequences from various marine, terrestrial, and aerial microbiome data sets. To express these proteins and link them to a massively parallel sequencing-based readout, we employed the M13 hyperphage display system, as it facilitates efficient polyvalent display of proteins with disulfide bonds²⁵⁻²⁷. Furthermore, polyvalency enhances binding avidity and thus sensitivity to detect lower affinity interactions. A large fraction of the known and related universe of cysteine-rich scaffolds and animal venom-derived polypeptides can therefore be encoded and displayed in candidate drug discovery campaigns. Here we coupled single-round screening with a sequencing readout, which facilitates rapid identification of binding interactions, even those of rare library members that might otherwise be outcompeted in a multiple-round panning process typical of traditional phage display.

As an initial demonstration of this platform, we focused on two critical receptors with distinct structural conformations: human epidermal growth factor receptor (hEGFR) and Mas-related GPCR family member X4 (MrgprX4). EGFR has a set of well-studied ligands and can be expressed both on the cell surface and in the form of a secreted immunoglobulin fusion protein. MrgprX4 is a seven-pass transmembrane protein that must be expressed on the cell surface. Despite its critical role in pain and itch signaling, selective antagonists have yet to be developed. Our platform robustly rediscovered known and novel ligands for these receptors. Screening in the presence and absence of endogenous ligands provides clues about the potential binding modes of candidate ligands, which can then be assessed via standard biochemical assays. In this initial study, our approach led to the discovery of a human venom-like CDP, tissue factor pathway inhibitor (TFPI), with antagonist bioactivity on the MrgprX4 receptor.

As discussed, stable polypeptide structures, particularly those incorporating disulfide bonds, have been evolutionarily co-opted for myriad biological functions that range from signaling, to defense, to predation.¹⁻⁵

A particularly successful class of such molecules relies on multiple intramolecular disulfide bridges to lock the polypeptide chain into a single conformation. Such structures can be endowed with unique properties contributing to their often potent bioactivities. 6 Their confined structures render them resistant to proteolytic degradation, and thus longer half-life in circulation and/or tissues. Additionally, a relatively small hydrodynamic radius and biochemical properties can enable their penetration through tissue barriers, such as the endothelium and even the blood-brain barrier.⁴

The inherently low entropy of their constrained structures reduces the Gibbs free energy of target binding (often to channels and receptors), thereby result in extremely high affinities. Each of these properties can be favorable in the context of a therapeutic modality, and in fact many peptides have been formulated into blockbuster drugs. Examples include Exenatide, Ziconotide and Eptifibatide, etc^{4,7}.

Exenatide is an FDA approved agonist of GLP1 receptor derived from the venom of the Gila monster. Exenatide became the first of a highly successful class of drugs that act as agonists of the GLP1 receptor with its approval for the treatment of type 2 diabetes in 2005. Eptifibatide was derived from the venom of rattlesnake. It was approved in 1998 and has been used as an antiplatelet drug. Ziconotide is an inhibitor of voltage-gated calcium channels from the venom of cone snail. This peptide was approved in 2004 as s treatment of severe chronic pain.

Phage display is a powerful methodology to identify polypeptide binders from large libraries of displayed molecules.⁸⁻¹⁰

Programmable phage display incorporates high throughput oligonucleotide library synthesis to encode large libraries (e.g. hundreds of thousands or more) of desired polypeptides ranging in length up to about 100 amino acids or more. A large fraction of disulfide rich venoms and toxins can therefore be encoded and displayed on phage using state of the art DNA synthesis technology.

High throughput (or “next generation”) DNA sequencing is a powerful approach to characterize phage display libraries, both before and after “panning” (enrichment of binders within the population).

Sequencing libraries after a single round of panning can be used to detect even weak interactions with rare members of a library, which otherwise might be outcompeted during the typical many-round panning process of traditional phage display. We have referred to this as sequencing-assisted selection.

Sequencing of a phage library that is panned against complex cell surfaces, multiple related targets, even in the presence and absence of endogenous ligands, can be used to identify polypeptides with exquisite specificity or even distinct binding modes expected to impart a desired bioactivity.

Protein disulfide bond formation tends to require translation into an oxidizing environment. The widely utilized M13 bacteriophage has been particularly successful in displaying proteins with disulfide bonds, most notably scFvs and the Fab fragments of antibodies.^{9,11-13}

Traditional M13 phage display has utilized monovalent display or coupling the displayed peptide to all of the P3 protein, an apparatus which is critical for infection of E. coli and thus library propagation. Monovalent M13 particles carry either zero or one copy of the peptide, which can fail to detect lower affinity interactions. The full P3 fusion system suffers from library bias due to interference with propagation. The M13 hyperphage system is a polyvalent display that uncouples library propagation from polypeptide display. The examples which follow utilize the M13 hyperphage system, but other phage display systems also can be readily employed.

As an alternative to M13 bacteriophage display, the MIPSA system (Molecular Indexing of Proteins by Self Assembly) or PLATO system (ParalleL Analysis of Translated ORFs) could also be utilized.¹⁵ Any other display technology could also in principle be used.

We have employed programmable phage display libraries for panning against serum antibodies or monoclonal antibodies. Many of the methods developed previously, including sequencing analytics and downstream informatic pipelines, can in many cases be applied to the analysis of programmable phage displayed polypeptide libraries panned against cells and/or their receptors.¹⁶⁻¹⁹

In certain aspects, the M13 hyperphage system is used to display a diverse library of venom and toxin polypeptides to discover novel ligands for a receptor (EGFR).

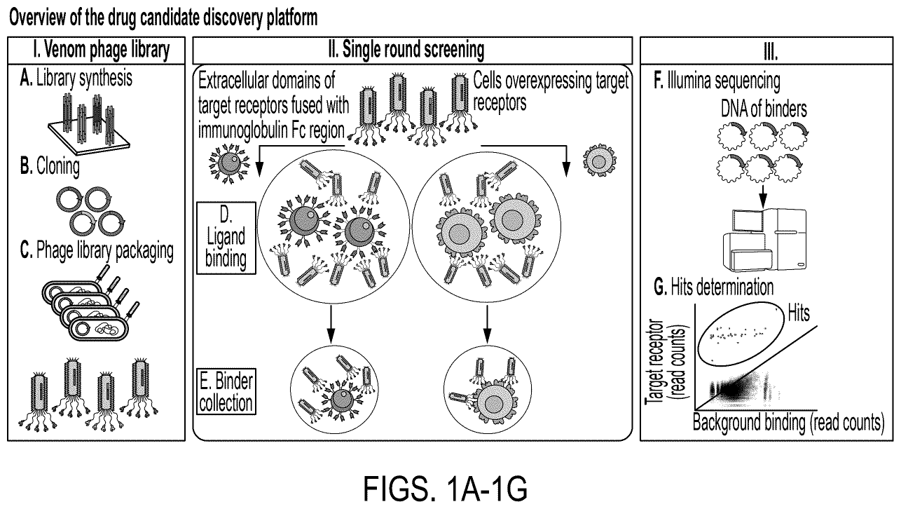

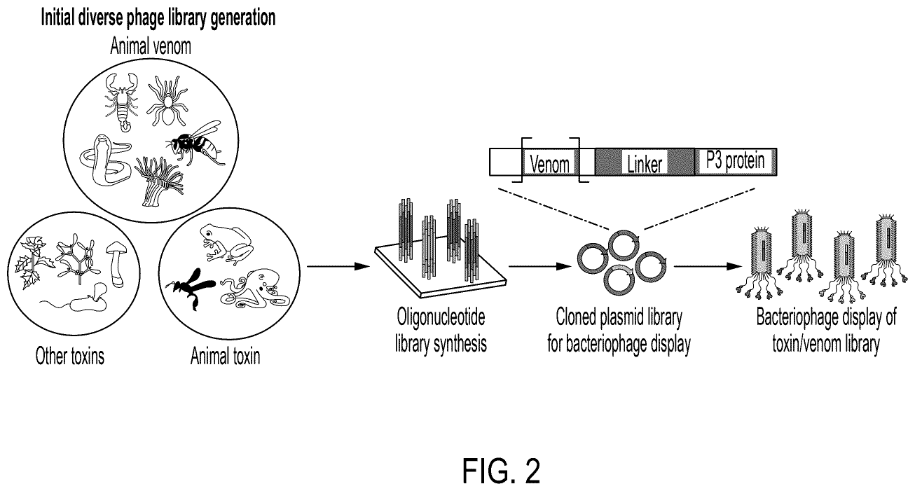

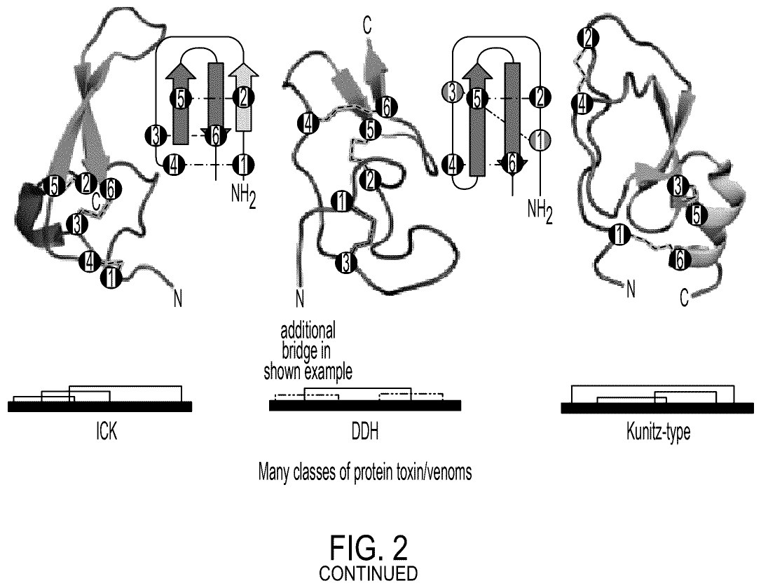

Referring now to the drawings, FIG. 1 depicts an oligonucleotide library synthesis scheme suitably used to identify sequences to encode polypeptides that are likely to display potent biological activities. This library can be comprised of venoms, toxins, poisons, or secreted human proteins for example. As shown in FIG. 1B, the oligonucleotide library is cloned in a single pot reaction into the M13 hyperphage phagemid and grown up in bacteria. As depicted in FIG. 1C, the bacteria are suitably infected with a “helper” phage that lack native P3 protein. The result is that all the P3 molecules produced in the bacterial host cells are derived from the phagemid construct and contain the desired N-terminally fused polypeptide. The encoding phagemid is efficiently packaged into the phage particle after infection with the helper phage.

As depicted in FIG. 1D, the phage library can be panned against cells that express a target on their surface (left), or against a target fused to a protein for affinity purification, for example an Ig chimera. After a single round of panning, unbound phage are washed away, thereby enriching the population for binders. See FIG. 1E. The enriched phage library, as well as the starting library and the library panned against control materials (e.g., cells that don't express the target or protein fusion tags alone) are sequenced to a depth that enables detection of differentially abundant library members. See FIG. 1F. By comparing each peptide's sequencing reads across populations, e.g., panned against cells expressing target versus cells not expressing the target, candidate binder peptides can be identified for further analysis. See FIG. 1F.

In one aspect, the present disclosure provides systems and methods for identifying or detecting a ligand in a sample. Such systems and methods include contacting at least one receptor with a sample comprising at least one toxin peptide; and determining whether a toxin peptide in the sample selectively binds to the at least one receptor, thereby identifying or detecting a ligand in a sample.

In some aspects, at least one receptor is expressed in cells, and the method includes transfecting the cells with a nucleic acid encoding the at least one receptor. The at least one receptor can include a receptor that is heterologous to the cell in which it is expressed. The at least one receptor can include a receptor that is native to the cell in which it is expressed. The at least one receptor can include a receptor that is stably expressed in a cell.

In some aspects, transfected cells include at least two cells comprising a first cell expressing a first receptor and a second cell expressing a second receptor different from the first receptor. In some embodiments, the at least one receptor is immobilized on a substrate.

The at least one receptor can include a transmembrane protein, e.g., a channel protein, e.g., a channel protein selected from the group consisting of a sodium ion channel, a potassium ion channel, a calcium ion channel, a chloride ion channel, a non-specific ion channel. In some embodiments, a transmembrane protein includes a potassium ion channel. In some embodiments, a potassium ion channel is a KvI.3 channel.

In some aspects, a sample includes a library of toxin peptides, e.g., a phage display library. In some embodiments, toxin peptides are about 5-200 amino acids in length. In some aspects, toxin peptides are about 5-100 amino acids in length. In some aspects, toxin peptides are about 5-50 amino acids in length. In some embodiments, toxin peptides are about 20-50 amino acids in length. In some embodiments, toxin peptides are about 30-50 amino acids in length.

Toxin peptides can include sequences found in a toxin that is naturally expressed in an organism (e.g., a snake toxin, a snail toxin, a scorpion toxin, a sea anemone toxin, a spider toxin, a lizard toxin). In some embodiments, a toxin peptide includes six cysteine residues.

In some aspects, spacing of the cysteine residues is conserved with the spacing of cysteine residues found in a natural toxin. In some aspects, a disulfide bonding pattern is conserved with a disulfide bonding pattern found in a natural toxin. For example, in some aspects, a toxin peptide includes at least 20, 25, 30, 25, 40, 45 or 50 amino acids, and has an amino acid sequence including at least six cysteine residues, so that the cysteine residues are located at each of the following positions within the 35 amino acids: 7 or 8, 13 or 14, 27 or 28, 32 or, 33, and 34 or 35. In some embodiments, a toxin peptide library includes toxin peptides in which a disulfide bonding pattern is conserved with a disulfide bonding pattern found in a natural toxin, and in which residues other than cysteines are altered (e.g., by randomization of residues at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 positions of a toxin sequence).

A toxin peptide library can include a plurality of unique toxin peptides, e.g., at least 10¹, 10², 10¹, 10⁴, 10⁵, 10⁶, 10?, 10⁸, 10⁹, 10¹⁰, 10¹¹, 10¹², or more unique toxin peptides.

The present methods and systems include identify, characterizing, and/or detecting ligands for receptors. In various embodiments, ligands are toxin peptides (e.g., toxin peptides derived from an animal venom). Provided methods can include the use of libraries of toxin peptides to permit simultaneous screening of multiple candidate ligand species. Methods herein are applicable to identifying ligands that derive from (i.e., are structurally related to) any toxin. For example, methods are applicable to toxin peptides which are derived from toxins of organisms such as sea anemone (e.g., Stichodactyla helianthus), scorpion (e.g., Androctonus mauretanicus, Odontobuthus doriae), snakes (e.g., Dendroaspis), spiders (e.g., tarantula), and snails.

Toxins that can serve as a scaffold for toxin peptides and libraries of toxin peptides include any of the toxins listed in a Table or Figure herein. For example, one or more of kaliotoxin, dendrotoxin, ShK toxin, hongotoxin-1, tarantula venom toxin vstxl, hanatoxin, fasciculin-2, dendroaspis natriuretic peptide (DNP), sarafotoxin, Odontobuthus doriae ODl toxin, Thrixopelma pruriens prototoxin-1, Thrixopelma pruriens prototoxin-2 can serve as a scaffold for generating a toxin peptide or library toxin peptides.

Ligand libraries for phage display can be generated by standard methods (e.g. Clackson and Lowman, Phage display. Oxford University Press, 2004; Barbas et al., Phage display. A laboratory manual. Cold Spring Harbor Laboratory Press, 2001).

In one example, ligand libraries for phage display with a combinatorial arrangement of ligand-domains are generated by designing overlapping or non-overlapping oligonucleotides corresponding to each individual domain. These oligonucleotides are phosphorylated, annealed, mixed in a desired combination and concentration and ligated into a phagemid vector with or without linker sequences to create a library by standard methods (Sambrook et al., Molecular Cloning: A Laboratory Manual. VoIs 1-3. Cold Spring Harbor Laboratory Press, 1989). For example, ligands may be composed by domains AlBlCl, A1B2C1, and A3B2C1, respectively. A combinatorial library of ligands in this representative example yields the pattern AnBnCn, where n is the i-th domain (for example, A2B1C3 is a novel ligand present in this library).

Library diversity can be verified by sequencing or by other physical, chemical or biochemical means, either with or without statistical analysis, that is suitable to use for diversity verification. Domains can be defined by functional, structural or sequence properties and can be of any length and a domain can be present or absent. Domains can be singular or highly varied to expand diversity.

In one example, a library includes toxin peptides (e.g., toxin peptides having sequences from one or more animal toxins). Toxin peptide libraries can include peptide animal toxins in their native or natural (wild-type) form or in any variation in amino acid sequence or may be comprised of DNA and/or RNA sequences encoding animal toxins. The library may contain toxins representing one or more scaffolds (also known as toxin-types or toxin families). For example, in some embodiments, a library includes toxin peptides from scorpion toxins, venom three-finger molecular scaffolds, or animal toxins that interact with K⁺ ion channels irrespective of the toxin's scaffold and species origin. In some embodiments, a library includes all known toxins from a given species, or all known toxins from all species.

In one example, an animal toxin library may include toxins from one or more of sea anemone, scorpion, and snake. Sea anemone Stichodactyla helianthus ShK toxin is pharmacologically active and blocks KvI.3 K⁺ channels in mammalian cells when expressed on the phage.

In some embodiments, a library is incorporated into a phage display system. In phage display, candidate ligands (e.g., toxin peptides) are functionally displayed on the surface of the phage and nucleic acid sequences encoding the ligands are enclosed inside phage particles. The functional display permits the selection of ligands that interact with a target or targets. A selection can be based on the ligand type (e.g., toxin type) and/or target biochemistry, pharmacology, immunology and/or other physicochemical or biological property. For example, a K⁺ channel toxin can be identified by screening scorpion toxin library on a K⁺ channel for binding to the channel.

In one example, a toxin peptide library is constructed and maintained such that each toxin peptide is individually constructed and stored and can be mixed into the library in any desired combination for the test to be performed. In another example, two or more toxin peptides may be constructed in the same reaction and stored and used together.

A phagemid library can be transfected into E. coli or other suitable bacterial species, propagated and the phages purified. At this stage, ligands (e.g., toxin peptides) can be functionally expressed on the surface of the phage and physically linked to their respective genes inside of the phage particle. A library is brought into contact with a target. After incubation with the target, those phages that express a ligand with no or weak recognition for the target are washed away. The remaining ligands that interact with the target can be (i) genotyped to establish the ligand identity, or (ii) processed for one or more rounds of panning, or (iii) otherwise quantified and/or identified (e.g., ELISA, microbiological titering, functional testing).

A ligand library (e.g., toxin peptide library) may be created by any known method. For example, a toxin library may be created by collecting peptides and/or nucleic acids encoding animal toxins and, if desired, non-venom homologues. Non-venom homologues include any molecule present outside of a venom gland or not used as a venom component but similar in sequence or structure to toxins. In applications employing phage display, N-terminal and C-terminal nucleotide sequences can be designed to join sequences for subcloning into a phagemid or other phage-display compatible vector. Overlapping or nonoverlapping DNA oligonucleotides are designed and synthesized for synthetic genes. This includes positive and negative DNA strands and N-terminal and C-terminal joining regions. The respective DNA oligonucleotide pairs (positive and negative strands) and sets are phosphorylated and annealed to create full length genes (e.g., genes encoding toxin peptides) with or without joining regions. Ligation into phagemid or other phage-display compatible vector is performed, for example using coat protein III as a fusion protein. Other suitable phage proteins may also be used. The sequences or genotypes can be confirmed.

In some embodiments, a linker sequence used to connect a ligand (e.g., toxin) sequence to a signal peptide and/or a coat protein of a phage (for phage display methods) and/or to any other domain is varied to optimize one or more of ligand expression, binding, or function. Varied sequences can be produced by any method.

In some embodiments, a phage library is contacted with and allowed to bind to the target of interest. To facilitate separation of binders and non-binders in the selection process, it is often convenient to immobilize the receptor on a solid support, although it is also possible to first permit binding to the target receptor in solution and then segregate binders from non-binders by coupling the receptor to a support. Bound phage may then be liberated from the receptor by a number of means, such as changing the buffer to a relatively high acidic or basic pH (e.g., pH 2 or pH 10), changing the ionic strength of the buffer, adding denaturants, adding a competitor, adding host cells which can be infected, or other known means.

In some embodiments, receptors are purified prior to ligand selection.

In another example, receptors may be expressed in cells. For example, cells may be stably or transiently transfected with one or more receptors can be utilized as expressed in native tissues.

Panning may be performed by the binding of ligands to the receptors, followed by washes and ligand recovery. In one example, panning is performed according to standard methods of phage display. Panning may be repeated until the desired enrichment is achieved. In addition, libraries can be pre-depleted on surfaces or cells that contain no receptors or on a receptor where the putative ligand receptor domain may be directly or indirectly altered. Additionally, any and all conditions of panning may be varied, altered or changed to achieve optimal results, such as the isolation of a specific ligand. Panning variations include, but are not limited to, the presence of competing ligand(s), presence of excess target(s), length and temperature of binding, pre-absorption of the ligand library on one or more different receptor(s) or cells or surfaces, composition of binding solution (e.g., ionic strength), stringency of washing, and recovery procedures. Phages recovered from panning may be processed for further rounds of panning, functional analysis, and/or sequencing/genotyping to deduce the resulting ligands' amino acid sequence or biological properties.

### Pharmaceutical Compositions

Compositions are also provided that include one or more of the present ligands. In particular aspects, pharmaceutical compositions are provided that include one or more of the present ligands, optionally together with one or more pharmaceutically acceptable carriers.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

### Kits

Kits are also provided as disclosed herein.

In one aspect, a kit is provided for identifying candidate bioactive compounds comprising a diverse toxin-like polypeptide display library comprised of multitude scaffolds.

In one aspect, a kit is provided for identifying candidate bioactive compounds comprising a diverse toxin-like polypeptide display library comprised of multitude scaffolds.

In another aspect. kits are provided identifying candidate bioactive compounds that comprise a library of DNA encoding diverse toxin-like polypeptides from a multitude of scaffolds to be used for creating a polypeptide display library.

The container means of the kits will generally include at least one vial, test tube, flask or bottle.

### Treatment Methods

As discussed, methods for treating a mas-related G protein coupled receptor-mediated (MrgprX4) condition in a subject are provided and suitably comprise administering an effective amount of a compound comprising a Kunitz-type domain to the subject, thereby treating the G protein coupled receptor-mediated condition. In particular aspects, the mas-related G protein coupled receptor-mediated condition may be adverse drug reactions (e.g., Stevens-Johnson Syndrome (SJS)), pruritus including cholestatic pruritus and other chronic itch conditions, and autoimmune diseases (e.g. multiple sclerosis).

In a particular aspect, methods for treating a chronic itch condition or disorder in a subject are provided and suitably comprise administering an effective amount of a compound comprising a Kunitz-type domain to the subject, thereby treating the chronic itch condition or disorder.

Suitably, a subject may be identified as suffering from or susceptible to a mas-related G protein coupled receptor-mediated condition such as an adverse drug reaction (e.g., Stevens-Johnson Syndrome (SJS)), pruritus including cholestatic pruritus and other chronic itch conditions, and/or an autoimmune disease (e.g. multiple sclerosis), and the compound comprising a Kunitz-type domain is administered to the identified subject. Preferably, the subject is a human.

The administered compound suitably may be a Kunitz-type protease inhibitor class protein that comprises a Kunitz-type inhibitor domain, which can be characterized by six cysteines forming a distinctive disulfide bond pattern of C1-C6, C2-C5, C3-C4. Specific Kunitz-type compounds that may be administered in accordance with these present therapeutic methods include TFPI (including human TPPI) and ERR1712142.

Derivatives of TFPI and ERR1712142 of suitably include a peptide whose partial amino acid sequence shows approximately at least 50%, 50-60%, 60-70%, 70-80%, 80%, 90%, 95%, 97%, 98%, 99% or 99.8% identity to TFPI or ERR1712142 and preferably demonstrates efficacy in an in vitro or in vivo assay of a present disease or disorder such as a chronic itch condition.

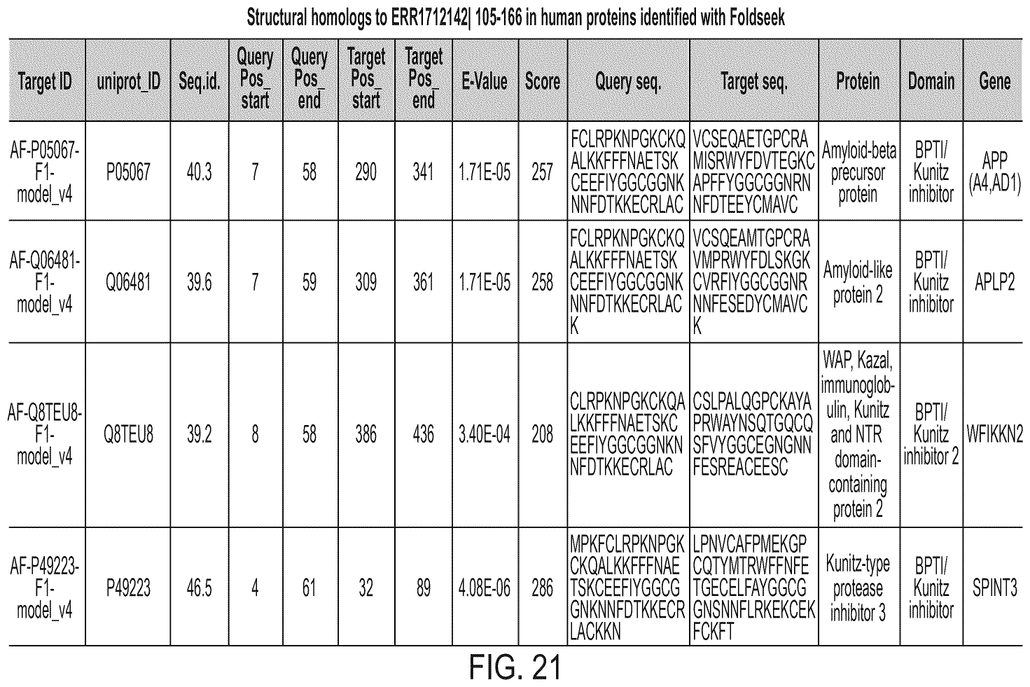

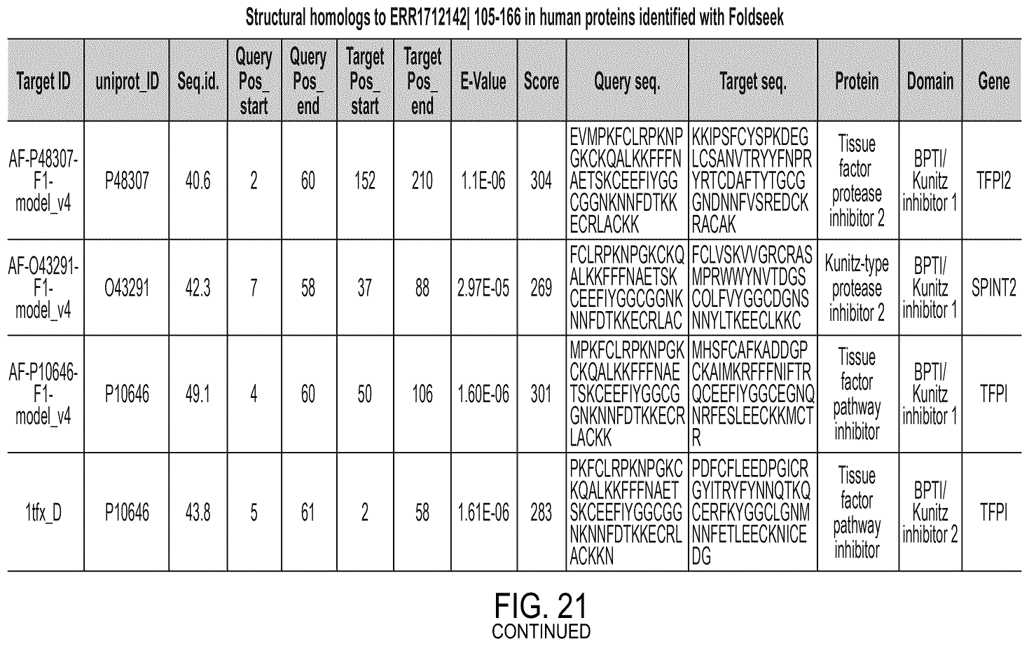

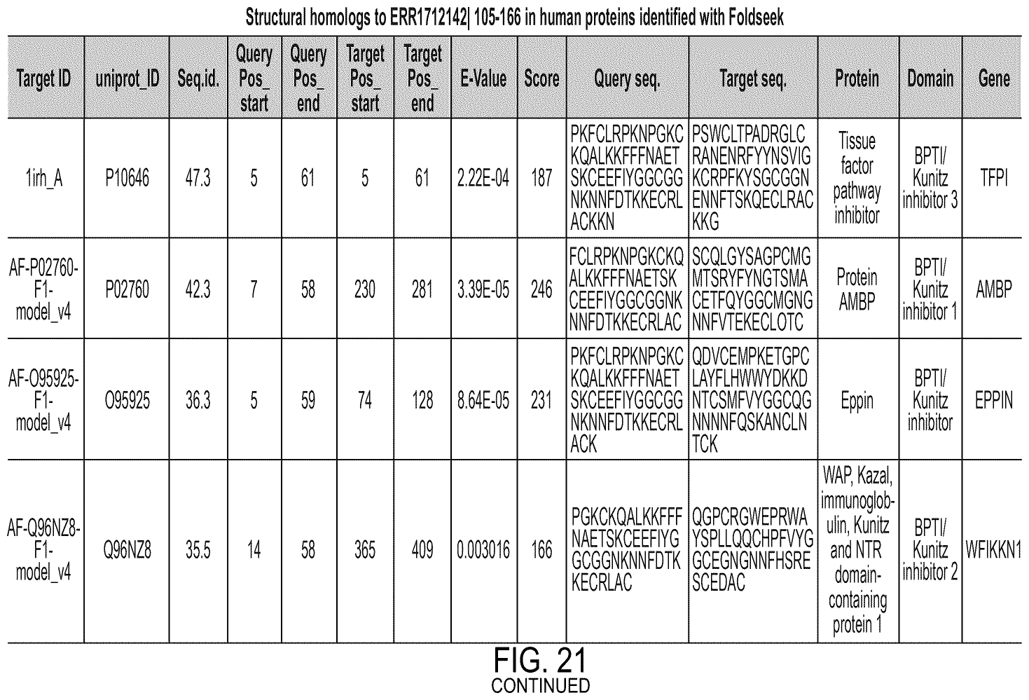

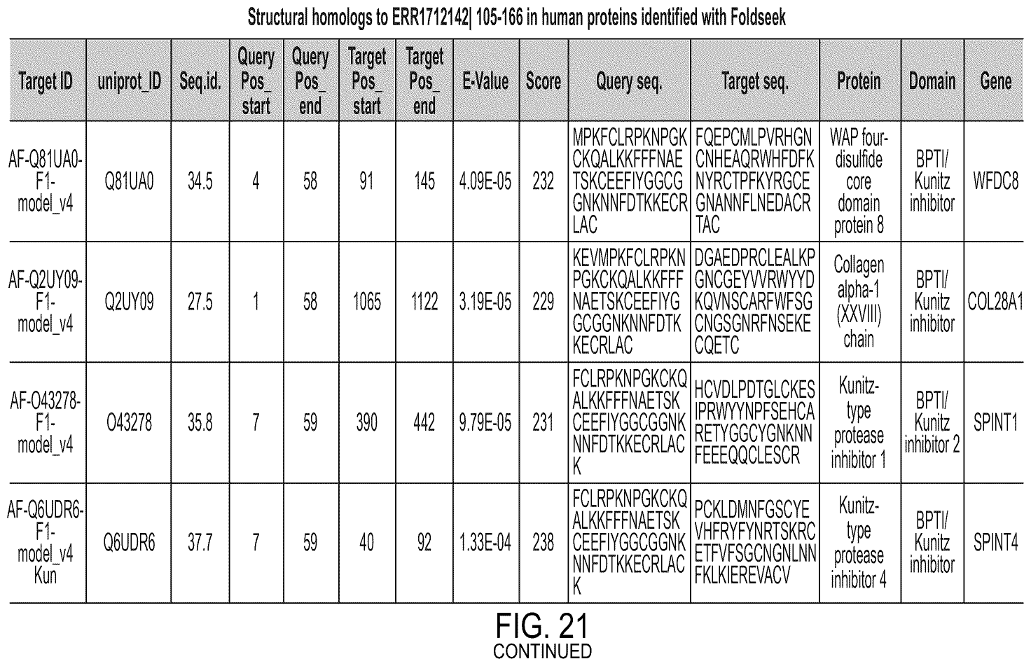

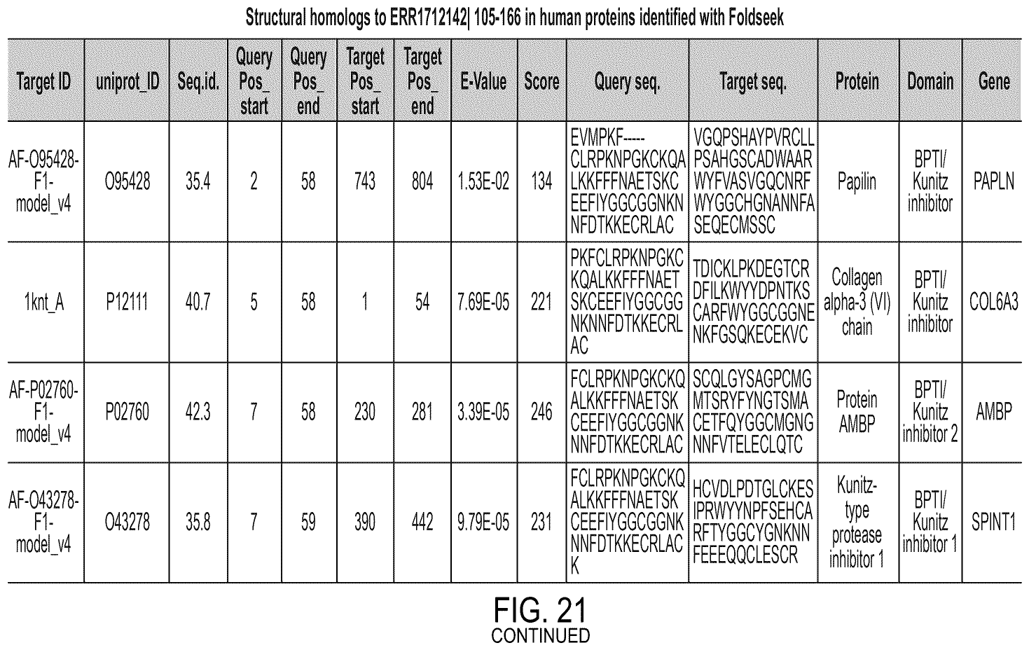

FIG. 21 also provides additional Kunitz-type compounds that may be administered in accordance with these present therapeutic methods.

The administered compound suitably may be a Kunitz-type protease inhibitor class protein that comprises a Kunitz-type inhibitor domain, which can be characterized by six cysteines forming a distinctive disulfide bond pattern of C1-C6, C2-C5, C3-C4. Specific Kunitz-type compounds that may be administered in accordance with these present therapeutic methods include TFPI (including human TPPI) and ERR1712142 and derivatives thereof. FIG. 21 also provides additional Kunitz-type compounds that may be administered in accordance with these present therapeutic methods.

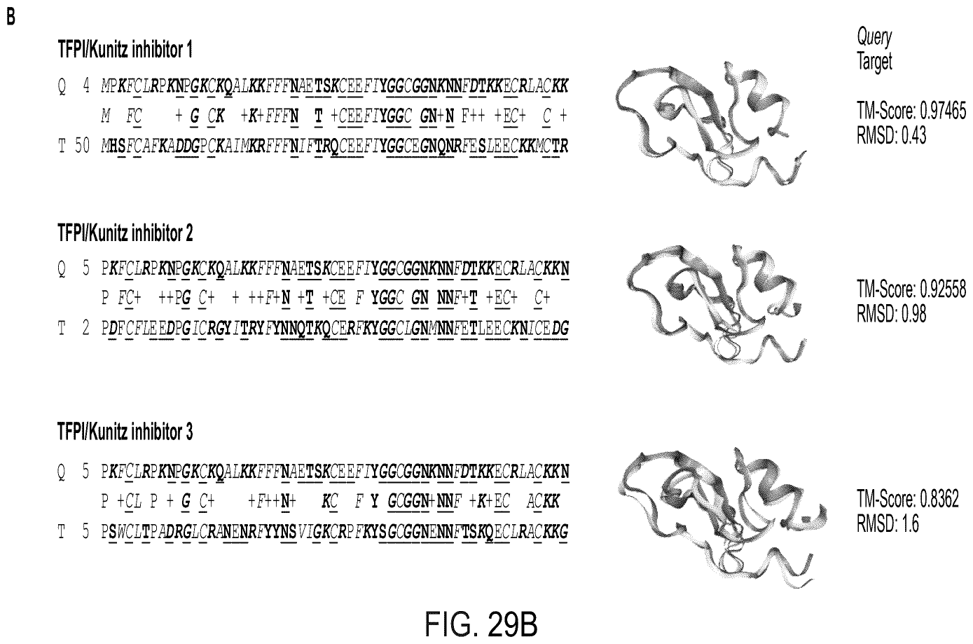

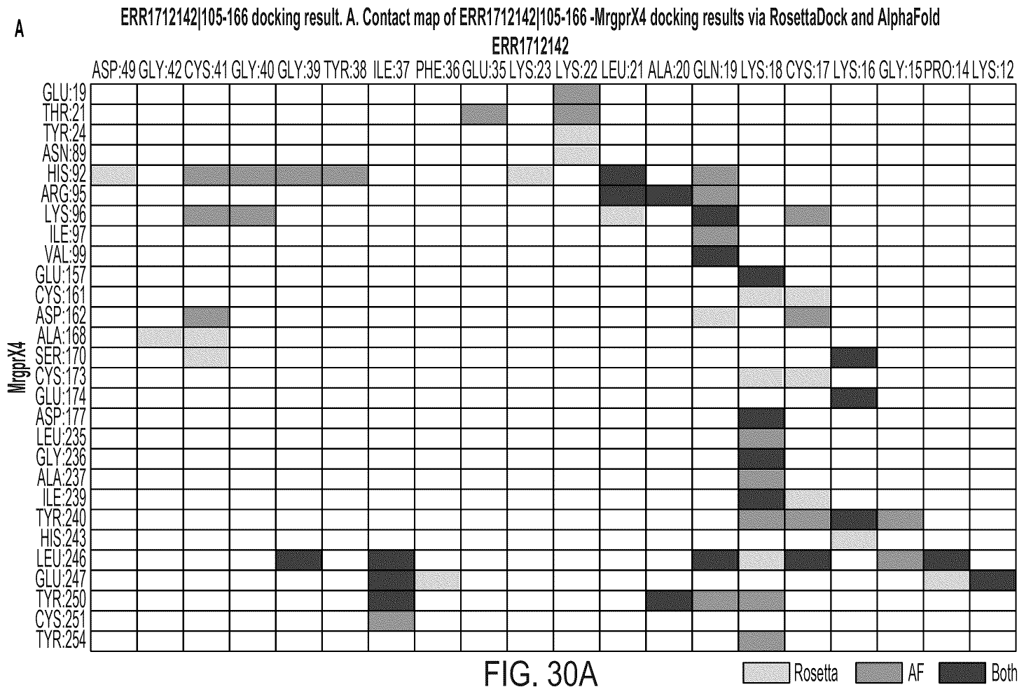

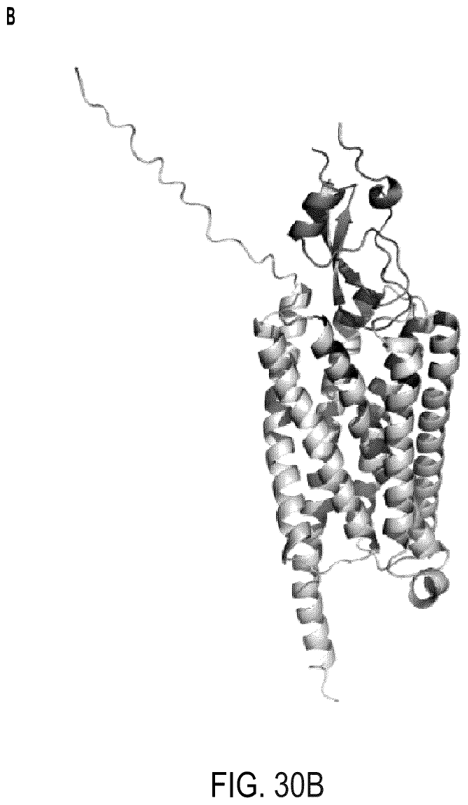

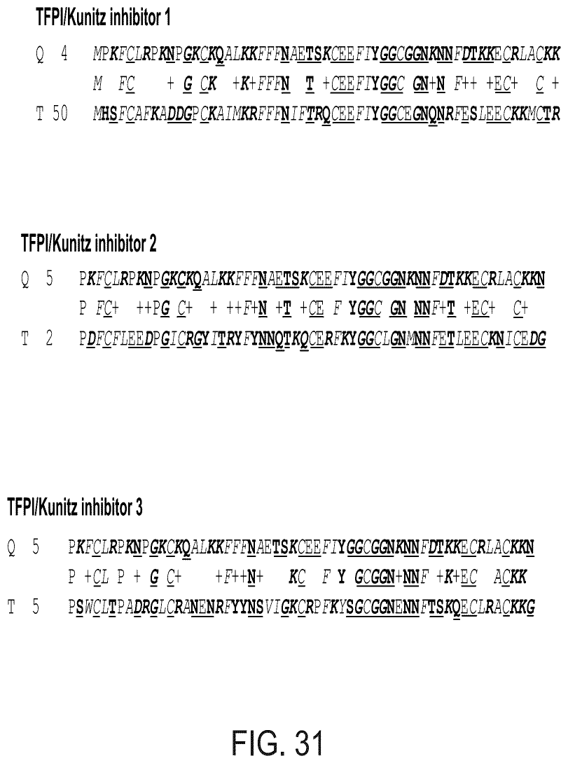

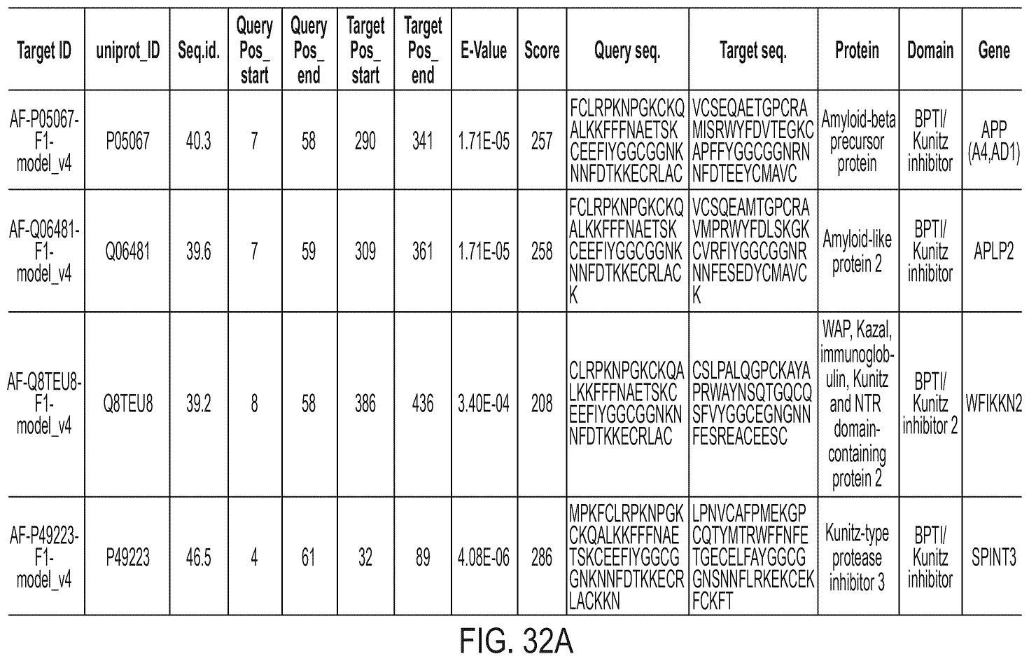

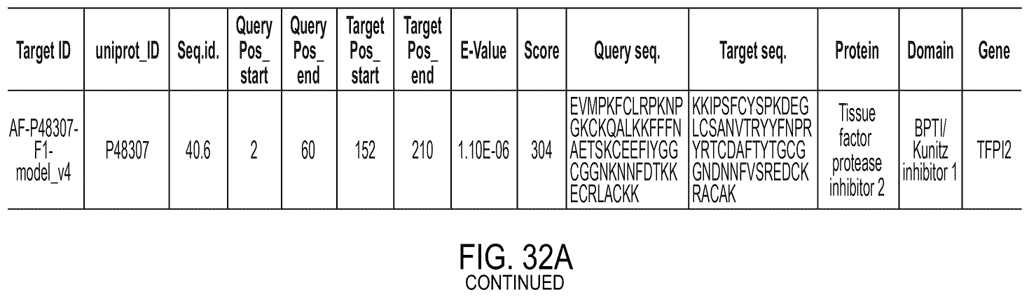

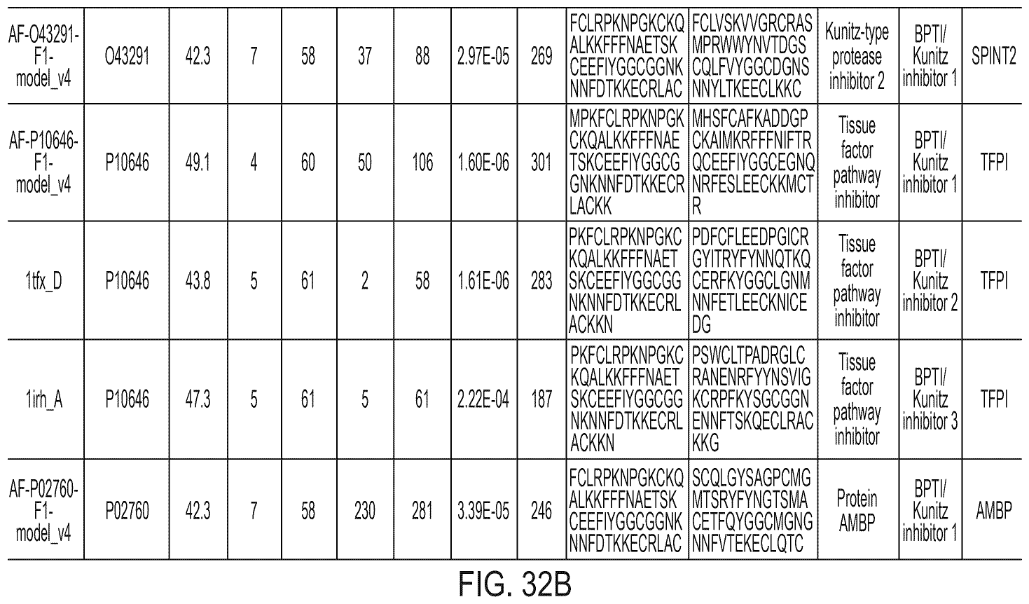

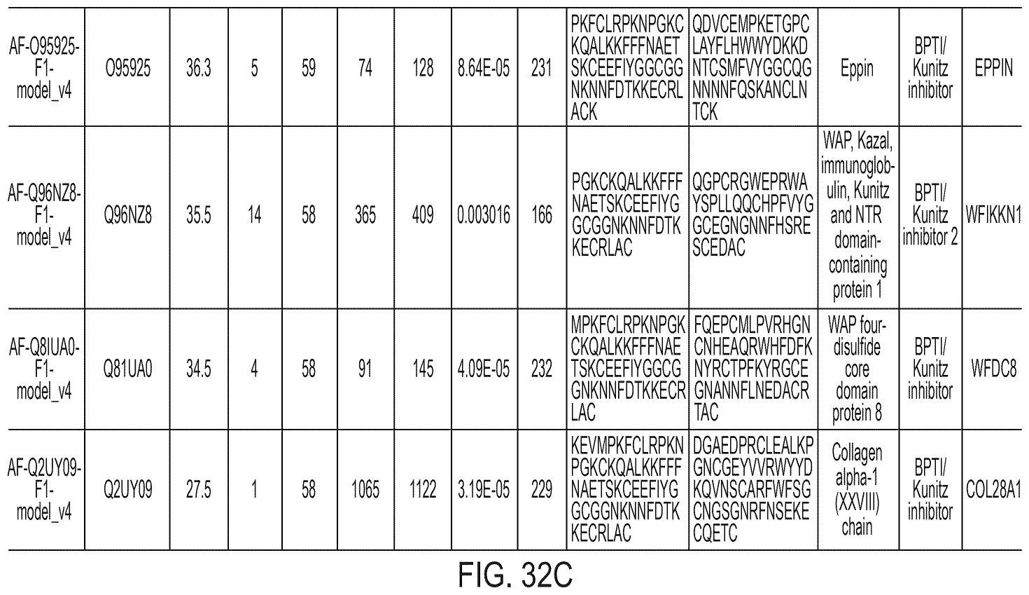

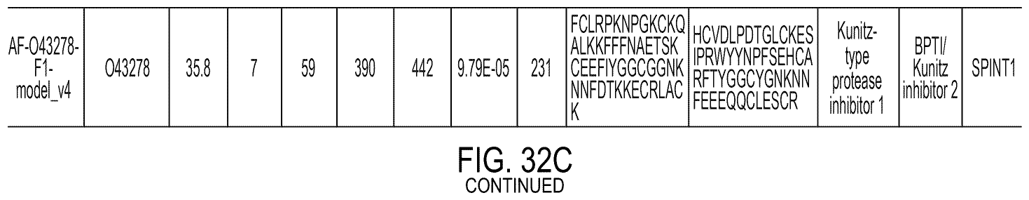

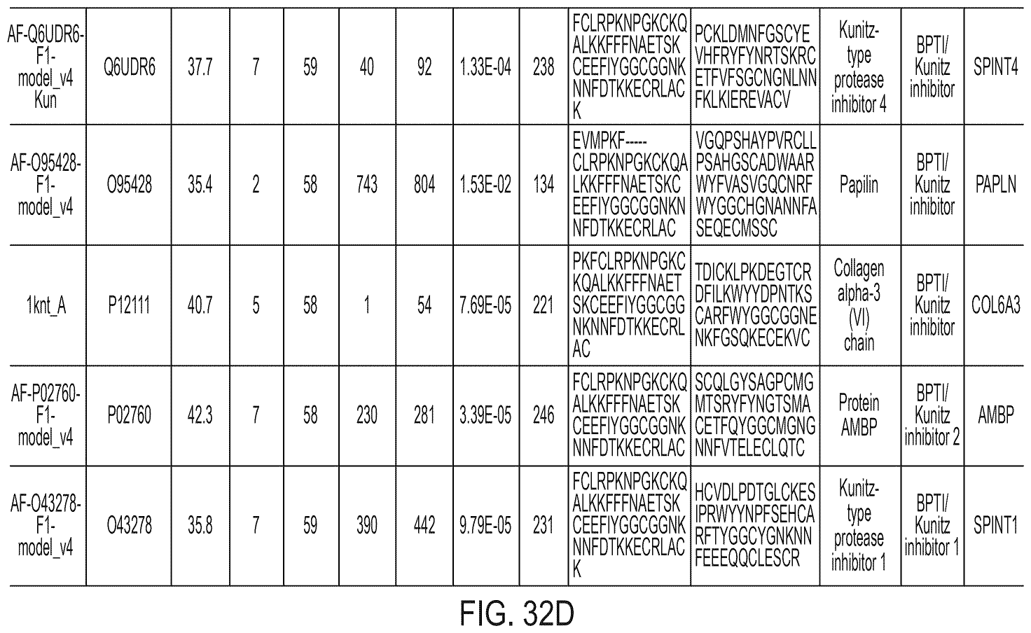

Preferred compounds for use in the present methods and pharmaceutical compositions include TPPI/Kunitz inhibitor 1, TPPI/Kunitz inhibitor 2, and TPPI/Kunitz inhibitor 3 (sequences set forth in FIGS. 29B and 31) and derivatives of each of those. Additional preferred compounds for use in the present methods and pharmaceutical compositions include the compounds or peptides set forth in FIG. 32A-32D and derivatives of those compounds.

Additional preferred Kunitz-type compounds are reported or are commercially available and can be identified empirically.

Further exemplary Kunitz inhibitor compounds that may be used in the present treatment methods and pharmaceutical compositions are disclosed in WO2014/144658; WO2022/055882; and US2004/0029312.

In certain aspects, preferred therapeutic compounds used in the present treatment methods and pharmaceutical compositions are peptides, including those peptide sequences and derivatives specifically disclosed herein.

As defined herein, a therapeutically effective amount of an agent (i.e., an effective dosage) depends on the agent selected. For instance, single dose amounts of an agent in the range of approximately 1 pg to 1000 mg may be administered: in some embodiments, 10, 30, 100, or 1000 pg, or 10, 30, 100, or 1000 ng, or 10, 30, 100, or 1000 pg, or 10, 30, 100, or 1000 mg may be administered In some embodiments, 1-5 g of the compositions can be administered.

A therapeutically effective amount of the compound of the present invention can be determined by methods known in the art. In addition to depending on the agent and selected/pharmaceutical formulation used, the therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for example, 50-500 mg/day, 50-300 mg/day, 100-20) mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, optionally, can include a series of treatments.

The following examples are illustrative only, and not limiting of the remainder of the disclosure in any way whatsoever.

### EXAMPLES

Animal venoms including related miniproteins exhibit unique structural and functional attributes for therapeutic development, such as high potency, target selectivity, and serum stability. However, the potential of these molecules remains largely underexploited, since the capability to screen diverse venom libraries in molecular display format has not been developed. We therefore established an approach to design and screen a comprehensive animal venom library, which was further sequence diversified to encompass the animal metavenome. Use of single-round M13 selection, coupled to high throughput next-generation DNA sequencing-based analysis, enables rapid discovery of lead molecules for further investigation.

Comprehensive screening of the animal venom, metavenome, and secretome libraries against EGFR unveiled binders associated with diverse molecular scaffolds. Alignment of the novel EGFR binders reveals conserved and divergent residues, information that may be used to guide further molecular diversification for candidate evolution. Sequencing-assisted selection can further guide candidate selection by directly measuring the impact of competing endogenous ligand. In the case of EGF, for example, our models predict that some candidate ligands, if further developed, may be able to interfere with EGF binding without agonizing the receptor. This approach can therefore accelerate the pace of discovering new peptide therapeutics with desired bioactivities.

In an effort to discover novel candidate therapeutics for cholestatic pruritus, we undertook a comprehensive screening campaign that identified six proteins that specifically bound MrgprX4, each of which appeared to contain a Kunitz-type protease inhibitor domain. Foldseek was then employed to search for candidate endogenous human scaffolds, with the potential to be developed into a therapeutic treatment with likely lower level of immunogenicity. One of these human proteins, tissue factor pathway inhibitor (TFPI), inhibited MrgprX4 activation at 300 nM in the presence of the MrgprX4 activator UDCA. However, MrgprX4 and TFPI exhibit a non-significantly overlapping pattern of tissue co-expression, suggesting that TFPI may be less likely to actually serve as an endogenous inhibitor of MrgprX4.

Single round, sequencing-assisted selection of venom libraries may be seemlessly integrated with machine learning approaches and generative artificial intelligence models to enhance library design and iteration³²⁻³⁴. Advanced structural models and docking algorithms will additionally provide powerful tools for scaffold engineering, as well as optimizing lead peptide drug candidates.

Despite its enormous potential, it is important to acknowledge certain inherent limitations of this drug discovery platform. First, the formation of disulfide bonds in E. coli cells may deviate from that in animal cells. Second, the M13 phage display system produces libraries with significant distributional skewing. The over- and under-representation of certain library members will reduce the fraction of the library actually queried in a screen. Mitigating library skew will be important for future platform improvement. Eukaryotic display systems such as yeast and mammalian display could be viable alternatives to the M13 phage display system. Fully in vitro systems like the Molecular Indexing of Proteins by Self Assembly (MIPSA)³⁵ or the ParalleL Analysis of Translated ORFs (PLATO)³⁶ might also provide certain advantages in specific settings. Being restricted to binding-based screening, however, a weakness common to all pooled screening approaches is their incompatibility with functional or phenotypic screening campaigns.

In summary, we have developed a drug discovery platform which unites polyvalent M13 phage display of cysteine-rich animal venom-derived libraries, single round sequencing-assisted selection, and cutting-edge computational approaches. We demonstrate the utility of the platform to discover ligands for receptors expressed on intact human cells. Specifically, we describe novel EGFR binders and a candidate endogenous antagonist of the pain and itch sensor MrgprX4. This drug discovery platform is therefore of broad utility for diverse classes of target molecules.

### Example 1: Animal Toxin Library

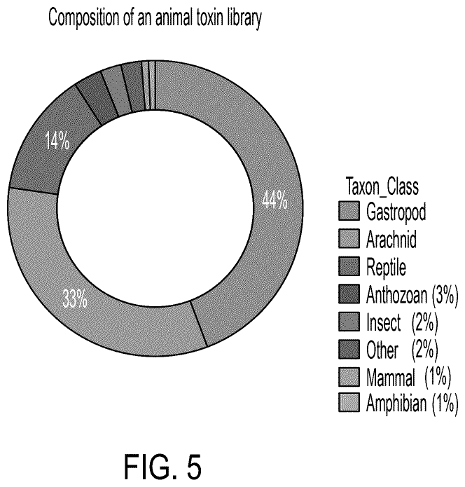

A highly diverse animal venom/toxin library was constructed from taxonomically and structurally annotated protein sequences in the Uniprot database. The relative composition of the library by taxon is shown in FIG. 5. Wherever possible, mature functional chain sequences were extracted according to protein structure annotation. In many cases, the known target of the toxin is also captured, which can help prioritize or deprioritize candidate molecules.

### Example 2: Animal Toxin Phage Library and Expanded Diversity Library

Two libraries were constructed and characterized by sequencing.

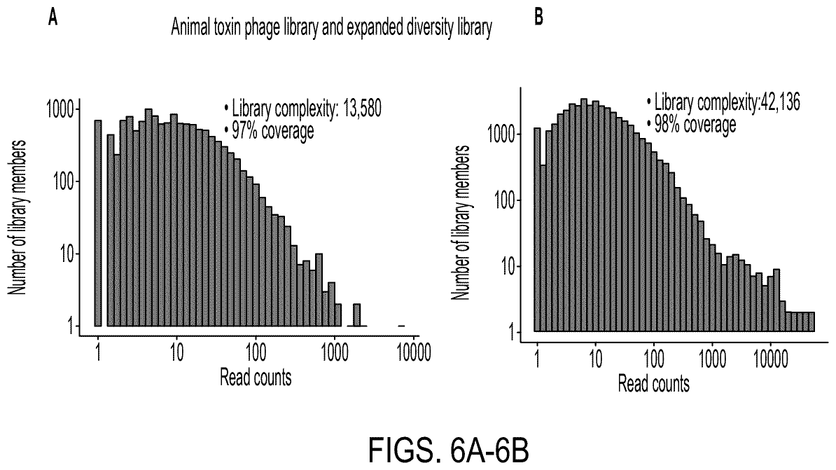

The animal venom/toxin library (composition presented in Example 1 above and FIG. 5), was cloned and packaged into the M13 hyperphage system. The designed complexity is 13,580. Sequencing revealed that about 97% of the intended library members were present in the final library. See FIG. 6A.

A second library of expanded sequence diversity, which was designed by searching for venom/toxin homologs in metagenomic databases, was cloned and packaged into the M13 hyperphage system. The designed complexity is 42,136. Sequencing revealed that about 98% of the intended library members were present in the final library. See FIG. 6B.

### Example 3: Ligands Discovered for Epidermal Growth Factor Receptor (EGFR)

Results of single round library screening against Ig-EGFR, Ig-EGFR in the presence of the endogenous ligand EGF, and against a negative control immunoglobulin (Isotype Ig).

In this experiment, M13 hyperphage libraries were used. A human secretome library was combined with the animal venom/toxin library (FIG. 5) and the expanded diversity library (FIG. 6). Similar to many toxins and venoms, the binding of EGF to the EGF Receptor requires proper disulfide bond formation, demonstrating that the M13 hyperphage system is appropriate to detect these types of interactions.

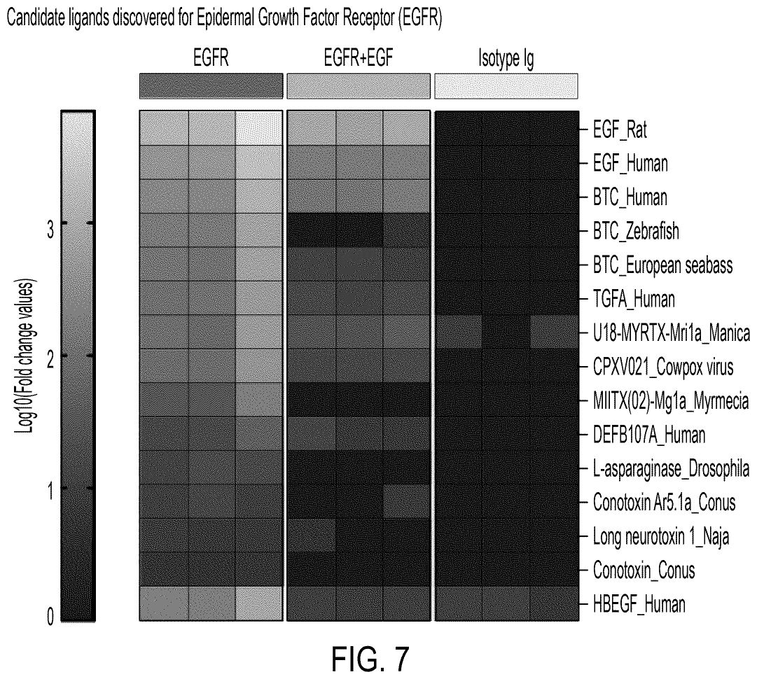

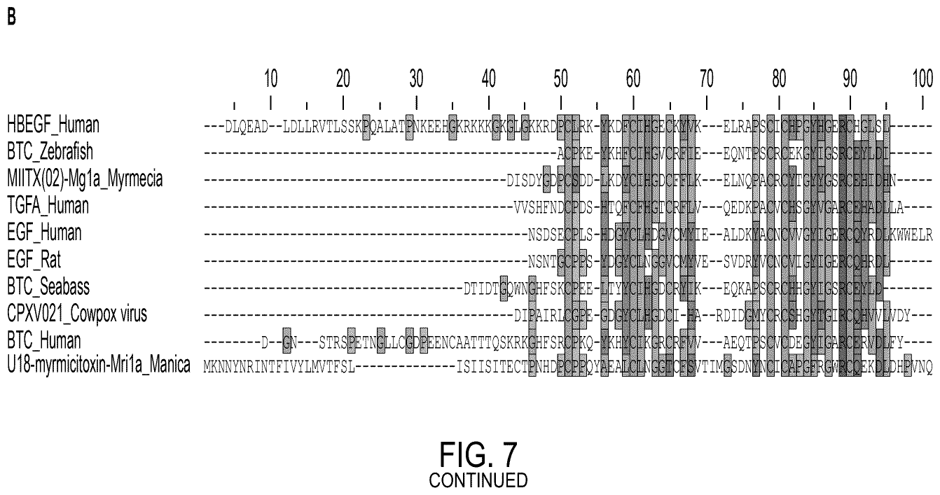

The top scoring polypeptides from the libraries are shown in FIG. 7A from triplicate screens in each condition. Fold change for each library member is calculated by comparison with the starting library. Most of the top scoring polypeptides were less strongly enriched by EGFR in the presence of the endogenous ligand EGF, suggesting that these candidates are likely to bind the receptor in a manner which is competitive with EGF.

In addition to human EGF, there are three more well-known high-affinity ligands for EGFR, transforming growth factor-A (TGFA), betacellulin (BTC), and heparin binding EGF-like growth factor (HBEGF). These ligands bind cell-surface EGFR with apparent Kd of 0.1-1 nM. Each of these known ligands was rediscovered using our system. Novel ligands are also represented in this list of hits, including conotoxins, which also appear to bind the receptor competitively with EGF.

Sequence alignment of the candidate hit list can reveal conserved sequence motifs, which can indicate a shared mode of binding. Identifying binding motifs can inform the design of a subsequent library with diversity focused on conserved or non-conserved residues, for example. See FIG. 7B.

### References Through Example 1

1. Peigneur, S. & Tytgat, J. Toxins in drug discovery and pharmacology. Toxins (Basel). 10, 10-13 (2018).
2. Pennington, M. W., Czerwinski, A. & Norton, R. S. Peptide therapeutics from venom: Current status and potential. Bioorganic Med. Chem. 26, 2738-2758 (2018).
3. Cole, T. J. & Brewer, M. S. Killer Knots: Molecular evolution of Inhibitor Cystine Knot toxins in wandering spiders (Araneae: Ctenidae). 1-18 (2021).
4. Smallwood, T. B. & Clark, R. J. Advances in venom peptide drug discovery: where are we at and where are we heading?Expert Opin. Drug Discov. 16, 1163-1173 (2021).
5. Pineda, S. S. et al. Structural venomics reveals evolution of a complex venom by duplication and diversification of an ancient peptide-encoding gene. Proc. Natl. Acad. Sci. U.S.A 117, (2020).
6. Pennington, M. W., Czerwinski, A. & Norton, R. S. Peptide therapeutics from venom: Current status and potential. Bioorg. Med. Chem. 26, 2738-2758 (2018).
7. Muttenthaler, M., King, G. F., Adams, D. J. & Alewood, P. F. Trends in peptide drug discovery. Nat. Rev. Drug Discov. 2021 204 20, 309-325 (2021).
8. Paschke, M. Phage display systems and their applications. Appl. Microbiol. Biotechnol. 70, 2-11 (2006).
9. Mimmi, S., Maisano, D., Quinto, I. & Iaccino, E. Phage Display: An Overview in Context to Drug Discovery. Trends Pharmacol. Sci. 40, 87-91 (2019).
10. Smith, G. P. & Petrenko, V. A. Phage display. Chem. Rev. 97, 391-410 (1997).
11. Nixon, A. E. et al. Drugs derived from phage display From candidate identification to clinical practice Drugs derived from phage display. MAbs 6, 73-85 (2016).
12. Omidfar, K. & Daneshpour, M. Advances in phage display technology for drug discovery. Expert Opin. Drug Discov. 10, 651-669 (2015).
13. Hamzeh-Mivehroud, M., Alizadeh, A. A., Morris, M. B., Bret Church, W. & Dastmalchi, S. Phage display as a technology delivering on the promise of peptide drug discovery. Drug Discov. Today 18, 1144-1157 (2013).
14. Rondot, S., Koch, J., Breitling, F. & Dubel, S. A helper phage to improve single-chain antibody presentation in phage display. Nat. Biotechnol. 19, 75-78 (2001).
15. Credle, J. J. et al. Neutralizing IFNL3 Autoantibodies in Severe COVID-19 Identified Using Molecular Indexing of Proteins by Self-Assembly. bioRxiv 2021.03.02.432977 (2021) doi:10.1101/2021.03.02.432977.
16. Venkataraman, T. et al. Analysis of antibody binding specificities in twin and SNP-genotyped cohorts reveals that antiviral antibody epitope selection is a heritable trait. Immunity 55, 174-184.e5 (2022).
17. Larman, H. B. et al. Autoantigen discovery with a synthetic human peptidome. Nat. Biotechnol. 201129629, 535-541 (2011).
18. Xu, G. J. et al. Comprehensive serological profiling of human populations using a synthetic human virome. Science (80-.). 348, (2015).
19. Román-Meléndez, G. D. et al. Citrullination of a phage-displayed human peptidome library reveals the fine specificities of rheumatoid arthritis-associated autoantibodies. EBioMedicine 71, 103506 (2021).

### Example 4: Adaptation of Programmable M13 Hyperphage System to Ligand Discovery Via Sequencing Assisted Selection

### Methods

### M13 Phagemid and Linker Design

The M13 phagemid vector was derived from the pSEX81 phagemid (PROGEN, Cat No. PR3005) and modified to be compatible with next-generation sequencing (NGS) platforms (FIG. S1A). The protein of interest (POI) was cloned at the N-terminal side of the P3 protein with EcoRI and HindIII restriction sites, and a FLAG epitope was encoded downstream of the POI. To investigate the impact of linker length on ligand enrichment, a series of linkers with various lengths were incorporated between the POI and the FLAG tag. For the M13 and M13-30 constructs, a G4S linker was used directly after the POI. For the M13-50, M13-70, M13-181, and M13-291 constructs, combinations of PAS37-39 and G4S linkers were used to create various linker lengths between the POI and the FLAG tag. The specific linker compositions used in each construct can be found in Table S1.

### Animal Venom, Metavenome, and Human Secretome Library Design and Synthesis

To obtain human secreted protein sequences, we accessed the UniProt database and downloaded entries based on the following search criteria: “taxonomy: ‘Homo sapiens (Human) [9606]’ (goa:(‘extracellular space [0005615]’) OR goa:(‘extracellular region [5576]’) OR locations:(location:‘Secreted [SL-0243]’))”. Subsequently, we extracted mature and active sequences with annotations or labels containing “chain” or “peptide” in the “PTM/Processing” section. For DNA synthesis, we retained only sequences that were equal to or less than 90 amino acids in length, regardless of whether they have mature and active forms. In total, 880 sequences were identified, and these were reverse translated with the pepsyn library design software [ref]. Sequences less than 270 base pairs were filled with PAS linker such that the final DNA length was brought up to 300 bases when appending primer binding sequences GGAATTCCGCTGCGT and CCGAGCATTGGCACC to the 5′ and 3′ end, respectively. This systematic approach for extracting mature and active sequences from the UniProt database was also applied in the design and generation of an animal venom library.

The animal venom library, comprising full-length active (mature) animal venom and poison protein sequences, was obtained from the UniProt animal toxin database [ref: Uniprot annotation project ref] using the search terms: taxonomy:“Metazoa [33208]” (keyword:toxin OR annotation:(type:“tissue specificity” venom)). To retrieve mature and active sequences, we employed the same method as described for the human secretome library generation. We extracted sequences both with and without mature and active forms and reverse translated 10,604 unique proteins that were equal to or shorter than 90 amino acids into their corresponding DNA using the pepsyn library design software. Sequences shorter than 270 bp were supplemented with PAS linkers, and primer binding sequences GGAATTCCGCTGCGT and GTCGTGCCAGGGAAC were appended to the 5′ and 3′ ends, respectively, to achieve a final DNA length of 300 bases.

To generate a metagenomic library expanding from the animal venom sequences, we first retrieved a list of known animal venoms. We searched the UniProt database for entries containing the keywords “toxin” and “animal”. These toxins were used as annotated queries for our searches against metagenomic databases. We searched for homologous sequences in two databases: the Big Fantastic Database (BFD) and a subset of SRA experiments identified by Serratus containing many RdRp's assembled by PLASS. We used mmseqs with the following parameters: 3 iterations, high sensitivity, and a maximum of 1000 hits per sequence (--num-iterations 3-a-s7.5--threads 100--max-seqs 1000--split-memory-limit 1T). The combined number of hits from both searches was 18,184,892. We then filtered these hits to include only proteins that cover the cleaved regions of the venome to at least 90%. We inferred the cleaved region using the alignment start and end position and created a predicted cleaved region for each hit. By considering only those where the database sequence had a cleaved region annotation, this resulted in 4,424,507 pairs from 9,388 annotated queries and 227,857 metagenomic hits. Next, we removed any identical (predicted) cleaved target sequences to reduce redundancy, resulting in 2,924,870 pairs from 8,202 annotated queries and 739,724 metagenomic hits. We also removed predicted cleaved regions longer than 100 amino acids, due to the size restriction of the phage display method, leaving 1,380,546 pairs from 6,913 annotated queries and 381,128 metagenomic hits. To generate a diverse but small set of sequences for the library, we removed any metagenomic sequences with at least 50% sequence identity and a 95% overlap to a cleaved venom sequence by running a mmseqs search. This resulted in 865,100 pairs from 6,471 annotated queries and 333,787 metagenomic hits. To further reduce the size, we clustered the cleaved sequences using mmseqs cluster with -c 0.95--min-seq-id 0.5, which left 70,415 pairs from 5,424 annotated queries and 70,415 metagenomic hits. To counterbalance the venoms that were only paired with a few metagenomic sequences, we added back metagenomic sequences that were removed in the last step so that each venom had at least 5 metagenomic matches, resulting in the final set of 85,406 pairs from 5,941 annotated queries and 39,583 metagenomic hits. From the metagenomic hits, we further extracted 36,140 sequences that were equal to or shorter than 90 amino acids in length. To enhance sequence diversity, we included an additional 4,996 sequences without mature and active forms and 1,000 random sequences that passed the same filtering process described above. In total, 42,136 sequences were reverse translated and supplemented with PAS linkers. To achieve a final DNA length of 300 bases, consistent with the human secretome and animal venom libraries, primer binding sequences GGAATTCCGCTGCGT and GCCTGGAGACGCCAC were appended to the 5′ and 3′ ends, respectively. The entire pipeline is implemented in python, which can be accessed at https://github.com/steineggerlab/phagedisplav-venoms. The code after the BFD/Serratus search stage runs in about 15 min.

The sequences encoding the human secretome library, animal venom library, and the metagenomic library were all synthesized by TWIST Bioscience (San Francisco, CA) and subsequently cloned into the M13-70 phagemid vector with EcoRI and HindIII restriction sites.

### Library Cloning

M13-70 phagemid, serving as the vector for library cloning, was digested overnight with EcoRI and HindIII restriction enzymes (NEB Cat No. R3101, R3104). In order to dephosphorylate the ends, the digested vector was treated with phosphatase (NEB Cat No. M0525) for a brief 10-minute duration followed by gel purification with a 2% low melting temperature agarose (ThermoFisher Cat No. 16500100) in 1×TAE buffer.

The oligo pool, comprising all three libraries used in this study, was reconstituted in molecular biology-grade water to a concentration of 100 ng/L. A total of 10 ng of the reconstituted oligos was utilized for PCR amplification (Agilent Cat No. 600679). Two PCR reactions were carried out. In the first round of PCR, the desired library was selectively amplified by performing 10 cycles using primers with specific binding sequences unique to each library. The amplified PCR product was then column-purified (QIAGEN Cat No. 28104). Following this, 10 second-round PCR reactions were set up, with each reaction containing 30 ng of purified first-round PCR product. A total of 5 cycles were conducted in the second-round PCR to introduce adaptors required for the subsequent restriction digest. Finally, the PCR-amplified product was purified again and digested with EcoRI and HindIII restriction enzymes followed by gel purification.

An adequate number of ligation reactions were prepared, each containing 50 ng of DNA (comprising both vector and insert at a 1:3 molar ratio) and high-concentration T4 DNA ligase (NEB Cat No. M0202T). The ligation mix was incubated at 16° C. overnight and column-purified with molecular biology-grade water. To identify the inserts and evaluate the quality of cloning, 20 colonies from each library were picked individually and miniprepped for Sanger sequencing.

M13 Phagemid E. coli Stock Preparation

To ensure each library member was represented by a minimum of 100 colonies, the number of reactions for phagemid DNA library transformation into TOP10F′ electrocompetent cells (ThermoFisher Cat No. 44-0002) was calculated accordingly. The transformed cells were incubated in SOC media at 37° C. for 1 hour with shaking at 250 rpm, followed by plating on LB agar plates supplemented with carbenicillin (50 mg/mL), tetracycline (5 mg/mL), and 100 mM glucose. The plates were then incubated overnight at 30° C., and the phagemid library stock were then collected and stored in −80° C. with LB supplemented with 25% glycerol, carbenicillin (50 mg/mL), and 100 mM glucose. To generate a single clone phagemid construct, the same transformation and plating protocols were followed as for the library, with the exception of the 100-colony representation rule.

M13 Phage Expression, Purification, and Quantification For phage library production, the E. coli phagemid library stock, which covered at least 100-fold of its library complexity, was diluted in pre-warmed LB media supplemented with 100 μg/mL carbenicillin and 100 mM glucose to an OD600 of 0.1. The culture was then incubated at 30° C. until it reached an OD600 of 0.4. Upon reaching the desired OD, the cells were infected with hyperphage (PROGEN Cat No. PRHYPE) at a multiplicity of infection (MOI) of 20 and incubated at room temperature for 15-20 minutes without shaking. The cells were subsequently incubated at 30° C. with shaking at 250 rpm for 45 minutes. The hyperphage-infected cell pellet was collected by centrifugation at 4,000 g and resuspended in 2XYT media supplemented with 100 μg/ml carbenicillin, 50 μg/ml kanamycin, and 200 M IPTG to induce protein production. To facilitate phage library production, the cell culture was incubated for 16 hours at 30° C. with shaking at 230 rpm. The bacteria were then pelleted by centrifugation at 8,000 rpm and 4° C., and the phage library was collected by filtering through a protein low binding 0.22 μm filter. To remove any residual bacterial debris, the library underwent two rounds of centrifugation at 10,000 g for 5 minutes at 4° C.

Subsequently, the phage library was concentrated to a volume at least ten times smaller than its original volume and buffer exchanged with PBS using a 15 mL Amicon 50 kDa spin filter. A protease inhibitor (Millipore Cat No. 11836170001) was added to the PBS-based concentrated phage library. To determine the number of phage particles in the solution, 100 μL of the phage library was subjected to small-scale dialysis with DNAse I buffer (10 mM Tris-HCl, 2.5 mM MgCl2, 0.5 mM CaCl₂)) overnight at 4° C. The following day, DNAse I was added to the dialyzed solution and incubated at 37° C. for 10 minutes to digest any remaining single and double-stranded DNA in the solution.

Finally, DNAse I was heat inactivated at 75° C., and the solution was diluted 100-fold with molecular biology-grade water before quantifying the number of phage particles using quantitative polymerase chain reaction (qPCR).

### Metagenomic Library Annotation

In this study, we aimed to annotate protein information and assign taxonomic classifications for the metagenomic library using NCBI-BLAST+software (version 2.13.0). Peptide sequences went through a blastp search against the comprehensive NCBI non-redundant protein database (as of July 2022) to find similarities in sequence. Depending on the query length, we tailored the alignment parameters accordingly. For sequences that were equal or less than 30 amino acids in length, we employed the parameters: “-evalue 200000-max_target_seqs 10-max_hsps 1”. The e-value was adjusted to accommodate short sequences. For amino acid sequences ranging between 31 and 90, the parameters were: “-evalue 1e-3-word_size 6-max_target_seqs 10-max_hsps 1” (default parameters: “-evalue 10-word_size 3-max_target_seqs 500-max_hsps>=1”). All other blastp parameters were maintained at default settings. We had explored more sensitive parameters, such as smaller word sizes, but ultimately settled on the current parameters to achieve a fine balance between sensitivity and program runtime. The blastp output included alignment metrics, protein names and accession IDs, and taxonomy identifiers (TaxIds). Utilizing this output, we identified the best hits based on the lowest E-value and queried the corresponding TaxIds within Taxonkit⁴⁰ to annotate the complete taxonomic lineages.

### Cell Lines and Cell Culture

NCI-H358 cells were cultured in McCoy's 5A medium supplemented with 10% heat-inactivated fetal bovine serum (FBS) and 50 U/ml penicillin-streptomycin, while MDA-MB-468, MDA-MB-231 overexpressing EGFR, and HEK 293T cells overexpressing CXCR2 were maintained in complete DMEM culture media (high-glucose DMEM supplemented with sodium pyruvate, 10% heat-inactivated FBS, and 50 U/ml penicillin-streptomycin). All cell lines were incubated at 37° C., 95% humidity, and 5% CO2.

To generate HEK 293T cells overexpressing EGFR, a lentiviral transduction method was employed. The full-length EGFR gene was inserted into the pCDH lentiviral expression vector (Addgene), and the lentiviral particles were produced using the pPACKH1 HIV Lentivector Packaging System (System Bioscience Cat No. LA500A-1). In brief, 3E+06 HEK 293T cells were plated on a 10 cm dish and incubated overnight in Iscove's Modified Dulbecco's Media (IMDM, Thermo Fisher) containing 10% FBS and 2 mM L-glutamine. On the subsequent day, 2 μg of the pCDH plasmids encoding EGFR were co-transfected with the pPACK packaging plasmid mixture into HEK 293T cells, utilizing GeneJuice (Sigma Cat No. 70967) as the transfection agent. After 48 hours, the media containing EGFR lentivirus was collected, filtered through a 0.45 μm filter, and used for transduction of 1E+05 HEK 293T cells in a 24-well plate along with 8 g/mL polybrene (Sigma Cat No. TR-1003-G) in 500 μL of complete DMEM. Following transduction, the cells were subjected to centrifugation at 800×g for 30 minutes at 32° C. and incubated overnight at 37° C. under 5% C02 conditions in a humidified incubator. The next day, the culture media was replaced with fresh complete DMEM, and the transduced cells were collected 10 days post-transduction to evaluate EGFR expression using flow cytometry. Cells exhibiting the top 10% of EGFR expression were sorted, collected, and subsequently cultured in complete DMEM.

### Flow Cytometry Analysis of Receptor Expression Level

Flow cytometry was utilized to evaluate the expression levels of target receptors in harvested cells. Approximately 5E+05 cells were resuspended in 1 mL FACs buffer (1% BSA or 5% serum in PBS without Ca2+ and Mg2, containing 0.02% NaN3) and transferred to a FACs tube. Following centrifugation at 300 G for 5 minutes, the supernatant was discarded. Cells were then incubated with 1 μg of primary antibody diluted in 100 uL of FACs buffer. For EGFR and CXCR2 overexpressing cells, mouse anti-EGFR mAb (ThermoFisher Cat No. MA5-13070) and mouse anti-CXCR2 mAb (R&D Systems Cat No. MAB331) were used for primary antibody staining, respectively. Samples were then stained for at least 30 minutes on ice before washing with 2-5 mL FACs buffer. After two subsequent washes by centrifugation at 300 g for 5 minutes at room temperature, cells were incubated with goat anti-mouse Alexa Fluor 488 mAb (ThermoFisher Cat No. A11001) secondary reagent for at least 30 minutes ice, shielded from light. Following a final double wash with FACs buffer, cells were adjusted to a concentration of approximately 1×10\hat{ }6 cells/mL, and the expression levels of target receptors were analyzed using flow cytometry.

### Single-Round Ligand Discovery Screening and NGS Sequencing

Two distinct screening methods were employed to identify novel ligands: a cell-based system and a receptor-Fc chimera immunoprecipitation technique. Both approaches involved incubating phage libraries with target receptors, collecting binders, performing PCR, and determining candidate hits based on next-generation sequencing (NGS) results. To ensure statistical validation and reproducibility, each condition was represented by triplicate samples.

In the cell-based screening method, cells overexpressing target receptors were detached with cell dissociation buffer (Corning Cat No. 25-056-CI) and collected after passing through 40 um cell strainer (Corning Cat No. 431750). The collected cells were washed three times with ice-cold PBS containing 1% (w/v) BSA (PBSA) and then incubated separately with the phage library in 1.5 mL Eppendorf tubes containing 1% PBSA for 30 minutes at 4° C. to minimize non-specific binding. Approximately 1E+06 cells were subsequently incubated with the phage library in 1% PBSA for 4 hours at 4° C. on an end-to-end rotator. After washing the cells three times with ice-cold 1% PBSA, ssDNA of the bound phages was collected using a modified protocol from the Quick-DNA/RNA Miniprep Kit (Zymo Cat No. D7011). Briefly, after cell lysis and chromosomal DNA removal, RNAse A (ThermoFisher Cat No. EN0531) was added to digest cellular RNA at 37° C. for 30 minutes. Next, the RNAse-digested solution was mixed thoroughly with an equal volume of 40% molecular biology-grade ethanol for ssDNA phagemid capture. The standard column purification protocol from the Quick-DNA/RNA kit was then applied, and 15 μL of eluted ssDNA binders were stored at −20° C. for subsequent quantification or sequencing.

For the phage immunoprecipitation-based ligand discovery, 1 μg of EGFR-Fc (R&D Systems Cat No. 344-ER) and Etanercept (Mippipore Sigma Cat No. 185243-69-0) chimera proteins, representing the extracellular domain of the target receptor fuse to the Fc region of immunoglobulin G, was incubated with the pre-blocked phage library overnight at 4° C. The following day, 20 μL of protein G beads (ThermoFisher Cat No. 10009D) were added to the phage-chimera mixture and rotated for 4 hours at 4° C. to immunocapture (IP) all chimeras. The beads were then washed three times with PBS containing 0.01% NP-40 and stored in −80° C. before sequencing.

Binders from both screening methods were subjected to PCR for library insert amplification and sample-specific barcode incorporation. The PCR products were pooled and analyzed using Illumina sequencing. The protocol employed here is a standard PhIP-Seq protocol that has been described in detail (ref. Mohan et al., 2018). Briefly, a first PCR was performed with primers that flank the displayed peptide inserts, and a subsequent PCR added adapters and sample indexes for sing-end read dual index Illumina sequencing.

### NGS Data Analysis

Illumina sequencing FASTQ outputs were demultiplexed and mapped to the reference sequences through alignment. Perfect matches were quantified to generate a read count matrix, with rows representing polypeptides in the library and columns corresponding to samples including the targets of interest and relevant negative controls. Each sample, including the negative control, was replicated three times in order to validate the data and ensure reproducibility. For the cell-based screening method, the negative controls were cells that did not overexpress the target receptor. For the phage immunoprecipitation-based method, the negative controls were human isotype immunoglobulin G (hIgG). EdgeR software^{41,42} was adapted to calculate the maximum likelihood fold-changes and the p-values of differential abundance in order to assess the enrichment for each sample relative to the negative controls. We determined that a significantly enriched polypeptide (“hit”) should have a p-value less than 0.001, a fold-change of at least 5, and a read count of at least 15 in two of the triplicate samples (fold change values and p values were calculated with the EdgeR software). These criteria were established heuristically to achieve optimal sensitivity and reproducibility.

Sequence Alignment-Based Network and MSA Analysis Network graph analyses were performed using R 4.2.0, with the network graphs generated through the R iGraph software package. Enriched peptides for each receptor screened were chosen to create the corresponding network graphs based on their sequence homology. Connectivity between sequences was determined by aligning protein sequences with blastp, utilizing the rBLAST package interfacing with the ncbi-blast+(2.13.0) software. Alignment parameters included “-evalue 1e−3-max_hsps 1-seg no-soft_masking false-word_size 5-max_target_seqs 100000-comp_based_stats none”. In the network graphs, polypeptides were represented by nodes, and those sharing sequence similarity while meeting alignment thresholds (E value<0.001) were connected. The link width was proportional to the BLAST bit-scores. To explore potential conserved motifs, sequences from individual clusters with more than two peptides were extracted for multiple sequence alignment using the Clustal Omega tool provided by the European Molecular Biology Laboratory's European Bioinformatics Institute (EMBL-EBI). The same sequences were also employed for motif discovery using MEME Suite 5.4.1.

### Protein Production and Purification

The genes encoding the peptides of interest were synthesized by IDT and cloned into a pLicC-MBP vector, derived from pLicC-MBP-APETx2 (Addgene)⁴³. This vector features a native MalE signal sequence for periplasmic delivery, facilitating disulfide bond formation, a 6×His-tag for immobilized metal affinity chromatography (IMAC), a maltose binding protein (MBP) to improve solubility, and an enterokinase (EK) recognition site to release bioactive protein. The peptide of interest (POI) was cloned upstream of the stop codons and immediately downstream of the EK cleavage site, ensuring a clean N- and C-terminal product after EK digestion.

Plasmids were transformed into E. coli strain BL21 (λDE3) pLysS (Promega Cat No. Li 195) or SHuffle cells (NEB, Cat No.C3029J) for MBP fusion protein production following the manufacturer's instructions. Starter cultures were prepared by inoculating single colonies in 5 mL Luria-Bertani medium with 100 g/mL carbenicillin and incubating at 220 rpm and 37° C. (BL21 (QDE3) pLysS) or 30° C. (SHuffle cells) overnight. These cultures were diluted 1:200 into LB medium supplemented with carbenicillin and grown under the respective temperature and shaking conditions. Protein expression was induced by adding 0.5 mM IPTG when OD600 reached ˜0.5 and incubating overnight at room temperature. The following day, cells were harvested by centrifugation and the cell pellets were stored at −80° C.

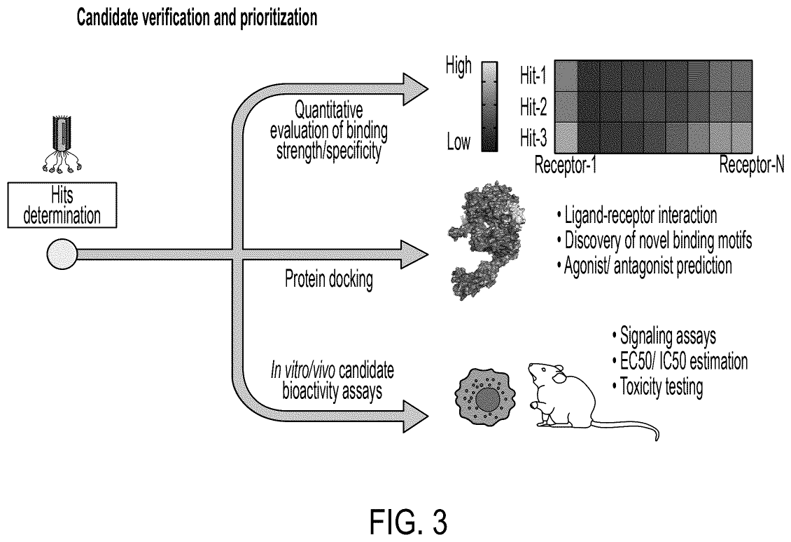

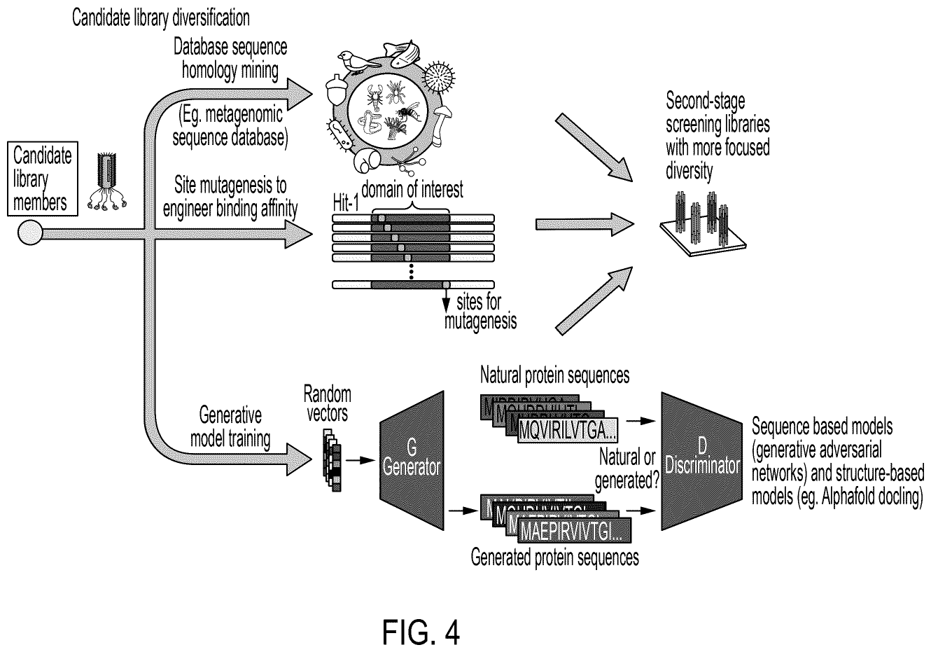

For whole-cell protein extraction, the cell pellets were thawed, resuspended in 1×TBS buffer supplemented with 10% glycerol and cOmplete™ EDTA-free Protease Inhibitor Cocktail (Sigma Cat No. 11836170001), and lysed by sonication at 22% amplitude for 3-5 rounds. In each round, sonication was applied for 30 seconds, followed by a 90-second rest on ice. The cell lysates were centrifuged at 30,000 g for 1 hour at 4° C. to remove cell debris completely. Fusion proteins were captured by passing the supernatant over gravity flow columns pre-packed with HisPur Ni-NTA resins (ThermoFisher Cat No. 88221). Non-specific binders were removed using a washing buffer containing 25 mM imidazole, and the proteins of interest were eluted with 150 mM imidazole. The procedure is depicted in Supplemental FIG. 3B. The eluted MBP fusion proteins were aliquoted and stored at −20° C. after concentration and buffer exchange into PBS using 15 ml Amicon 50 kDa spin filters (Millipore Cat No. UFC905024).

### Enterokinase Cleavage to Release Bioactive Protein

MBP recombinant proteins eluted from IMAC were concentrated and buffer-exchanged into EK cleavage buffer (20 mM Tris-HCl, pH 7.4, 50 mM NaCl, 2 mM CaCl₂)) for EK digestion. Enterokinase (GenScript Cat No. Z03004) was added to the protein suspension at a ratio of 1 unit per 50 pg protein, and the mixture was incubated at 4° C. with rotation overnight to allow EK cleavage. The following day, protein samples were collected and dialyzed in PBS using Slide-A-Lyzer™ MINI Dialysis Devices, 3.5K MWCO (ThermoFisher Cat No. 88403). The proteins were aliquoted and stored at −20° C. for subsequent assays or at 4° C. for quantification. FIG. S3C, D provide examples of two MBP fusion proteins pre- and post-EK digestion.

### Protein Quantification

Densitometry-based image analysis of protein gel staining was employed for protein quantification. MBP fusion proteins (approximately 50 kDa) were quantified using Precision Plus Protein Unstained Protein Standards (Bio-Rad Cat No. 1610363). The 50 kDa protein band containing 750 ng of protein per 10 μL loading volume served as the protein standard for quantification. Post-EK cleavage proteins were quantified with the Ultra Low Range Molecular Weight Marker (Sigma Cat No. M3546), with 100 ng/μL of aprotinin (corresponding to the 6.5 kDa band) used as the protein standard.

Serial dilutions of the designated molecular weight markers were included on 10-20% Tricine gels (ThermoFisher Cat No. EC66252BOX) to generate a standard curve for reference. For EK-released proteins, an additional 1-hour fixation step using fresh 5% glutaraldehyde solution was applied. Gels were stained with PageBlue™ Protein Staining Solution (ThermoFisher Cat No. 24620) for 1 hour at room temperature and de-stained with water for 2-3 hours before imaging. ImageJ software was utilized for image processing and densitometric analysis. Protein concentrations were determined by averaging the values from more than three distinct dilutions. An example of this quantification methodology is provided in FIG. S3E.

### Binding Assay for ERR1712142|105-166 Screening Against MrgprX4

pLicC-MBP-ERR1712142 plasmid was extracted from bacterial lysate with Maxiprep (Qiagen Cat No. 12662) after overnight incubation in BL21 strain BL21 (λDE3) pLysS. The plasmids were transcribed in-vitro using the HiScribe T7 High Yield RNA Synthesis Kit (New England Biolabs Cat No. E2050S) to produce RNA. The 40 μl reaction contained 500 ng plasmid template, 20 μl NTP buffer mix and 4 μl T7 RNA polymerase and was incubated at 37° C. for 2 hours. After transcription the product was diluted with 60 μl molecular biology grade water DNA and the plasmid was cleaved at 37° C. for 15 minutes by the addition of DNAse I. Then 50 μl of 1 M LiCl was added to the solution and incubated at −20° C. for 30 mintues. The centrifuge was cooled to 4° C., and the RNA was spun at maximum speed for 30 minutes. The supernatant was removed, and the RNA pellet washed with 70% ethanol. The sample was spun down at 4° C. for another 10 minutes, and the 70% ethanol removed. The pellet was dried at room temperature for 15 minutes, and subsequently resuspended in 100 μl water.

RNA of translated using the PURExpress ΔRibosome Kit (New England Biolabs Cat No. E3313S). The translation reactions contained 0.4 μM mRNA, 10 μl Solution A, 3 μl Factor Mix, 0.3 μM Ribosomes, 20 U Murine RNase inhibitor (Protector RNase inhibitor, Millipore Sigma Cat No. 3335399001), 1 μl of Disulide Bond Enhancer 1 and 1 μl of Disulfide Bond Enhancer 2 (New England Biolabs Cat No. E6820S). The reactions were incubated at 37° C. for 8 hours and used immediately or stored at −80° C. 2.5 μl of the translated product was run on a 4-12% SDS-PAGE (Thermofisher Scientific Cat No. NP0321BOX) gel and transferred onto a PVDF membrane and stained for validation of translation.

HEK293-MrgprX4 and HEK293 cell lines were detached with cell dissociation buffer (Corning Cat No. 25-056-CI) and collected after passing through 40 m cell strainer (Corning Cat No. 431750). The collected cells were washed three times with ice-cold 1% PBSA. The cells were then incubated with ligand prepared in 1% PBSA for 4 h at 4° C. on an end-to-end rotator. After washing the cells three times with ice-cold 1% PBSA, the cells were washed one more time with FACS washing buffer (lx PBS, 0.05% azide, 2% FBS) and then incubated with fluorescence-conjugated FLAG tag antibody (PE, BioLegend Cat No. 637309, 1:150 dilution; or Alexa-647, ThermoFisher Cat No. MA1-142-A647, 1:400 dilution) for 10-15 min at room temperature in the dark. The cells were then washed three times with ice-cold FACS washing buffer and resuspended in 400 ul buffer for the following flow cytometry analysis. Flow cytometry analysis was conducted on Beckman Coulter CytoFLEX. Data from flow cytometry was processed on Flowjo software.

### EGFR Cell Signaling Assay

For the EGFR signaling assay, MDA-MB-468 cells were seeded in 6-well plates (6E+05 cells/well) and starved in serum-free DMEM medium. After 20 hours, cells were stimulated with 3.2 nM commercial EGF (R&D Cat No. 236-EG-200), 5 nM EK-released TX21 and monkeypox proteins for 30 minutes respectively, then lysed using RIPA buffer (ThermoFisher Cat No. 89900) with protease and phosphatase inhibitor cocktails (ThermoFisher Cat No. 1861278, Sigma Cat No. P0044). Supernatants were collected for Western blot analysis after centrifugation at 14,000 g for 15 minutes. Protein samples were separated on NuPAGE™ 4-12% Bis-Tris precast gels (Invitrogen, Cat #NPO322BOX) and transferred to PVDF membranes using the iBlot™ Gel Transfer System (Invitrogen, Cat #IB401032). Membranes were blocked with 5% non-fat dry milk in TBST (1×TBS, 0.1% Tween-20) at room temperature for 30 minutes, then incubated with primary monoclonal antibodies targeting pErk (Thermo, Cat #14-9109-82; 1:5000 dilution) or GAPDH (R&D, Cat #MAB5718; 1:10000 dilution) overnight at 4° C. or for 4 hours at room temperature. After washing with TBST, membranes were incubated with HRP-conjugated anti-mouse IgG (Cell Signaling, Cat No. 7076S; 1:2000 dilution) for 1 hour at room temperature in the dark. Protein detection was achieved using a chemiluminescent kit (Cytiva, Cat #GERPN2236).

### MrgprX4 Calcium Signaling Assay

The HEK293-MrgprX4-GFP and HEK293 cell lines were dissociated with 0.25% Trypsin (Gibco, Cat No. 25200-056) and collected after 1-2 minutes incubation. The harvested cells were then seeded at a density of 2.5E+04 cells/well in a 96-well half-area flat clear bottom black plate (Greiner Bio-One, Cat No. 675090) that was pre-treated with 0.1 mg/ml poly-D-lysine. The following day, cells were stained with FLIPR Calcium 5 dye (Molecular Devices, Cat No. R8186) in a loading buffer composed of Hanks' balanced salt solution (HBSS) with 20 mM HEPES, adjusted to pH 7.4. After a 30-minute incubation period at 37° C., TFPI (Acro Biosystems Cat No. TFI-H5226) was introduced, and the cells were incubated for an additional hour at 37° C. Subsequently, UDCA or the buffer control were automatically dispensed into the wells, which were immediately imaged using the Flexstation 3 system (Molecular Devices).

### (a) Docking Prediction

We employed the AlphaFold-Multimer implementation in ColabFold version 1.5.2, incorporating templates from pdb70, to perform docking of four targets on EGFR (EGF, MIITX(02)-Mg1a, omega-HXTX-Hi2g_2, and phospholipase A2 homolog) and ERR1712142|105-166 on MrgprX4. AlphaFold-Multimer generated five docked models for each target, of which we selected the top-ranked model, guided by the average pLDDT scores. We further subjected the top-ranked model to RosettaDock 4.0, using conformational ensembles on ROSIE. From the 1000 models generated by RosettaDock, we selected the top 10 based on interface scores. To identify key amino acids involved in binding, we generated contact maps of the top-ranked models from both AlphaFold and RosettaDock using MAPIYA, considering the distance between amino acids with a cut-off set at 5 Å⁴⁴.

To discover novel interactions involving animal venom-derived ligands with diverse structural characteristics, we first optimized a programmable M13 polyvalent “hyper” phage display screening platform²⁵. The M13 phage's minor coat protein p3 is secreted through the E. coli periplasm where disulfide bond formation can occur, thus facilitating proper folding of the fused venom-like polypeptides.

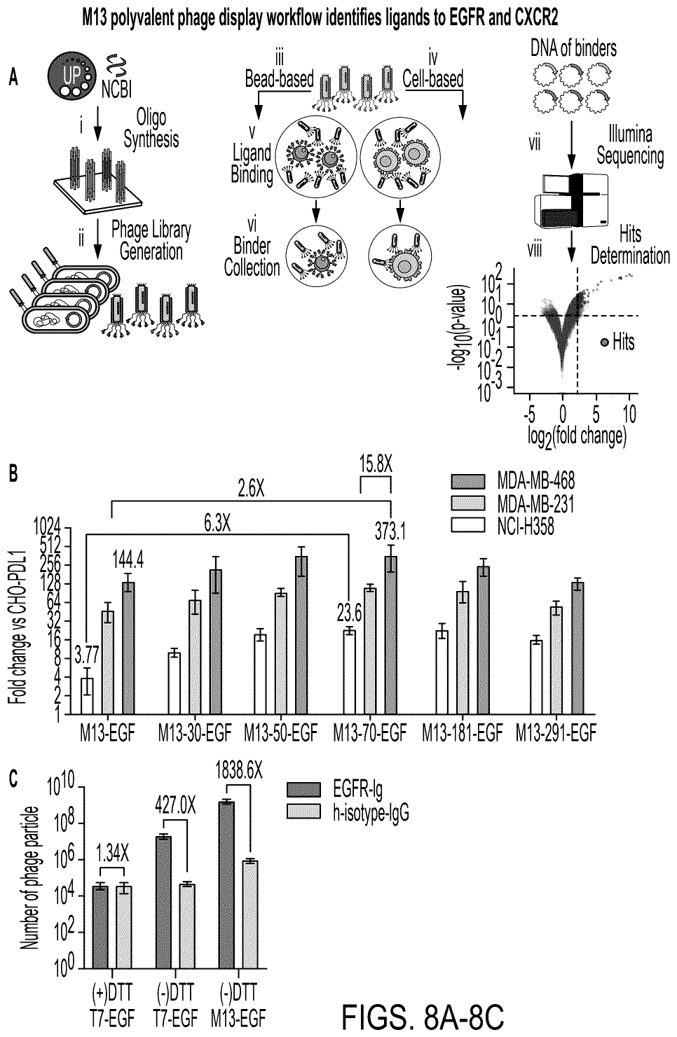

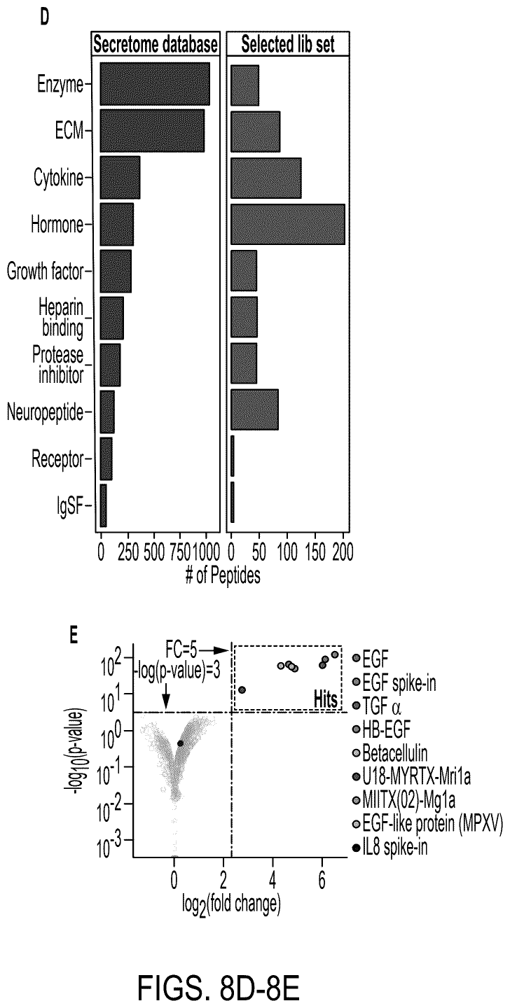

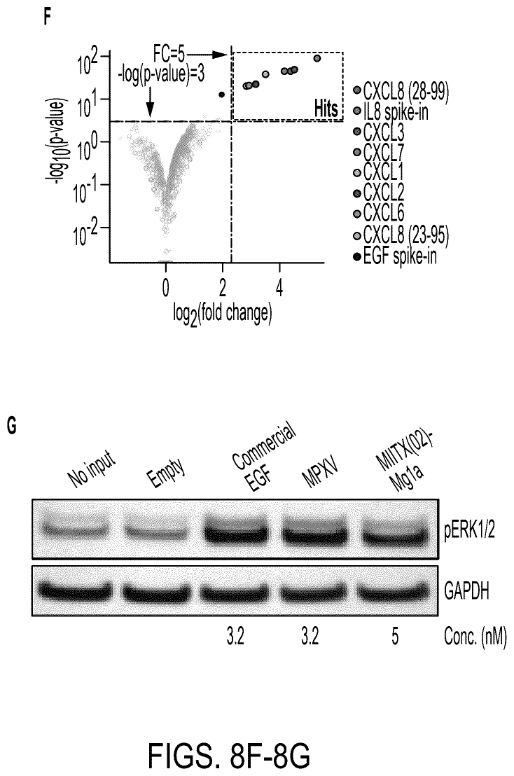

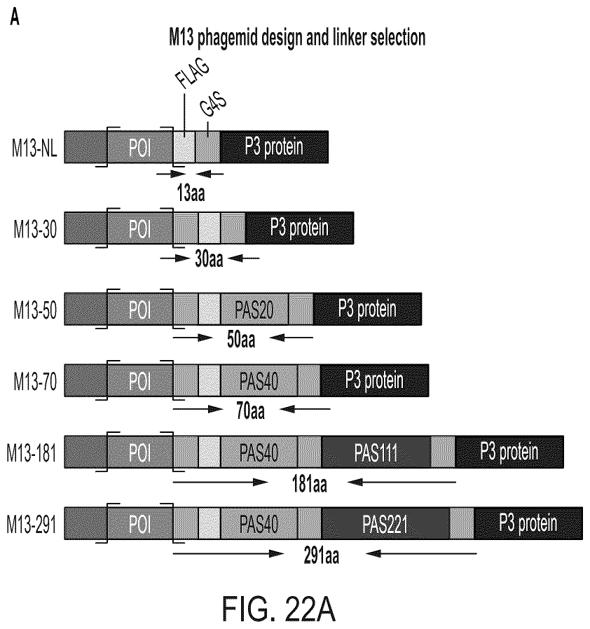

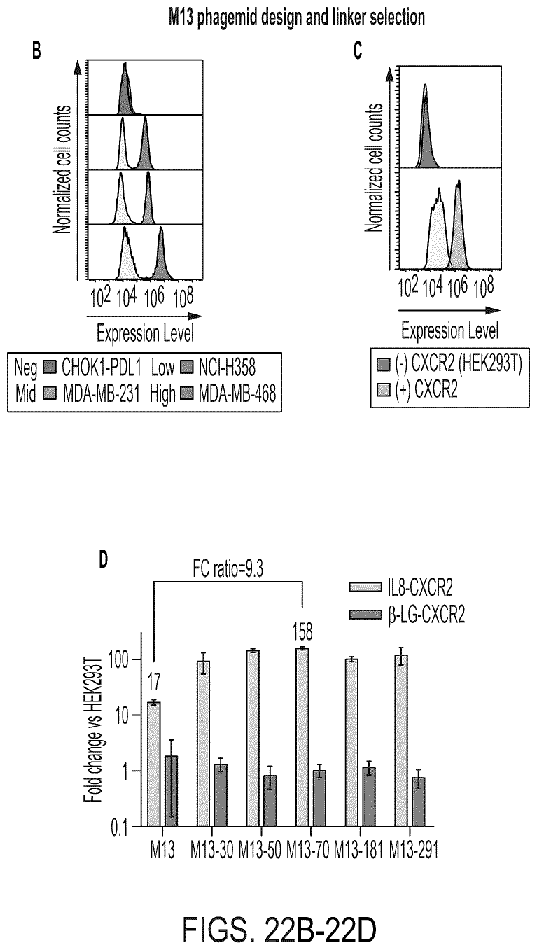

The M13 screening process consists of three main steps: phage library construction, single-round selection, and candidate identification via library sequencing-based analysis (FIG. 8A). To optimize ligand selection, varying linker lengths were tested in the context of M13-displayed epidermal growth factor (EGF) binding to native human EGFR (hEGFR) expressed at different levels. The EGF-hEGFR interaction is high affinity (Kd-0.1-1 nM)²⁸ and absolutely requires EGR's three disulfide bonds. A range of linker lengths between 13 to 291 amino acids (FIG. 14 and FIG. 22A) were tested in conjunction with three cell lines of varying EGFR expression: high expression (MDA-MB-468), medium expression (MDA-MB-231), and low expression (NCI-H358). As a negative control, we used CHO K1 cells overexpressing PDL1, which are known to have low levels of EGFR expression (FIG. 22B).

The impact of linker length on cells with lower EGFR expression levels (NCI-H358) were significantly more pronounced compared to MDA-MB-468 cells, which exhibited the highest EGFR expression level. When adopting a 70 aa-long linker, higher fold changes were observed across all cell lines, with approximately 16-fold higher enrichment between MDA-MB-468 and NCI-H358 cells (FIG. 8B). A similar linker-length dependent trend was found when phages expressing interleukin-8 (IL8) were tested for binding to cells overexpressing the GPCR for IL8, C—X—C Motif Chemokine Receptor 2 (CXCR2) (FIG. 22C, D). Based on these findings, subsequent libraries were constructed in a phagemid vector incorporating the 70aa-long linker (M13-70).

In addition, we evaluated the importance of disulfide bond formation and its impact on the binding of EGF to EGFR by comparing the M13 and T7 phage display systems with a bead-based immunoprecipitation assay (FIG. 8C). T7 are lytic phages that do not use the periplasm for replication, thus forming disulfide bonds much less efficiently in their displayed peptides. EGF expressed on M13 phage demonstrated a much higher binding ratio compared to the T7 phage system. T7-EGF binding was ablated by disulfide bond reduction with DTT, while T7 phage integrity and infectivity were unaffected.

(b) Re-Discovery of Known Membrane Protein Receptor Ligands with Programmable M13 Polyvalent Phage Display of the Human Secretome

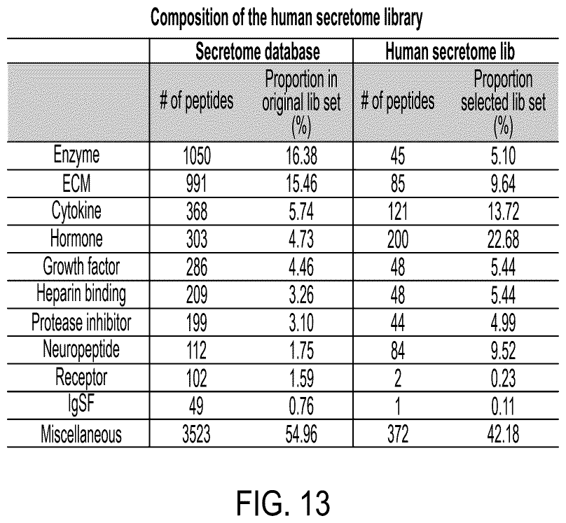

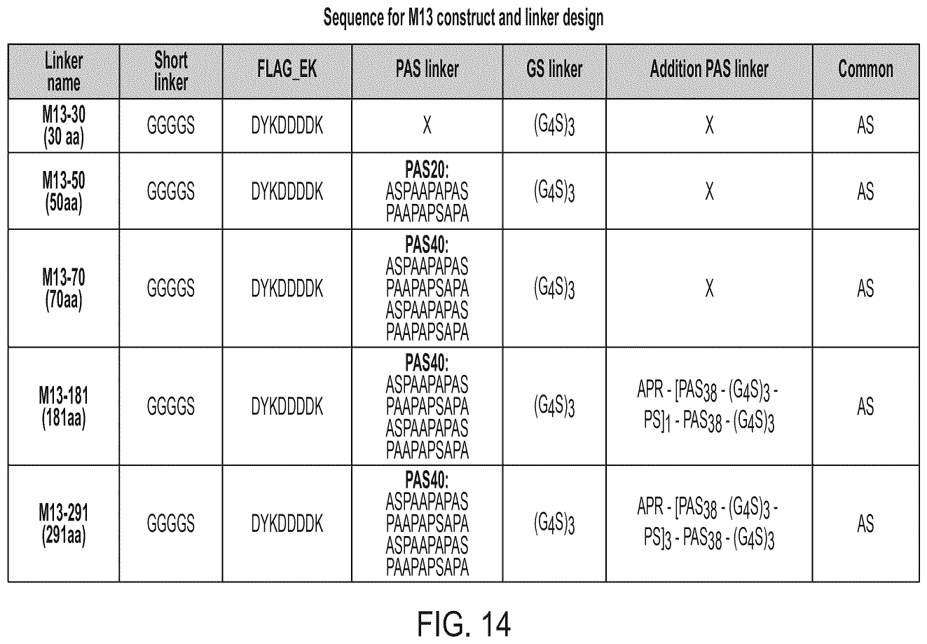

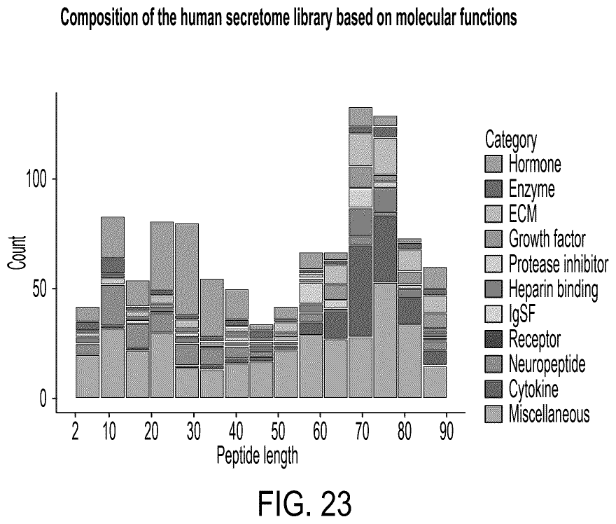

To further assess how well the optimized M13 polyvalent display system could identify diverse ligands for cell surface expressed receptors, we constructed a library encompassing all 880 full-length human extracellular and secreted proteins (the human secretome library) whose mature polypeptide chains are equal to or less than 90 amino acids in length. Our library includes approximately 66% of hormones listed in the Secretome database, including known EGFR ligands such as EGF, betacellulin (BTC), transforming growth factor-alpha (TGFa), and heparin-binding EGF-like growth factor (HB-EGF). We also incorporated three additional proteins as positive controls for EGFR studies, namely, MPXV, an EGF-like protein from the monkeypox virus, and two ant venoms, Mri 1a and Mg1a, which are known EGFR agonists. In addition to hormones, nearly 33% of cytokines and 75% of neuropeptides from the comprehensive Secretome database are included in the human M13 secretome library. Most extracellular matrix (ECM) proteins and secreted enzymes are not included in the library due to their sizes (FIG. 13, FIG. 8D). However, proteins included in the human M13 secretome library cover a diverse range of molecular functions, including hormones (200), enzymes (45), growth factors (48), protease inhibitors (44), and neuropeptides (84) (FIG. 23).

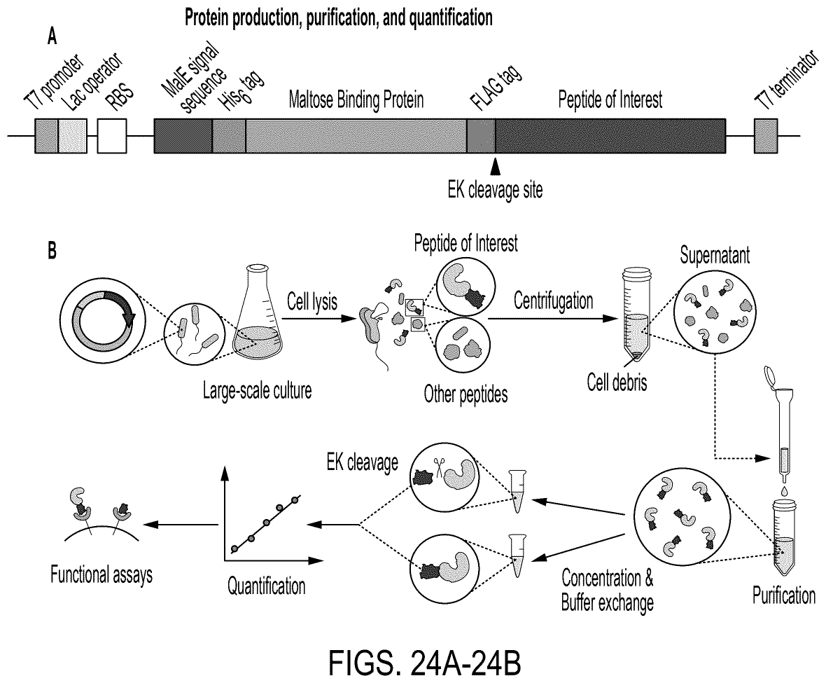

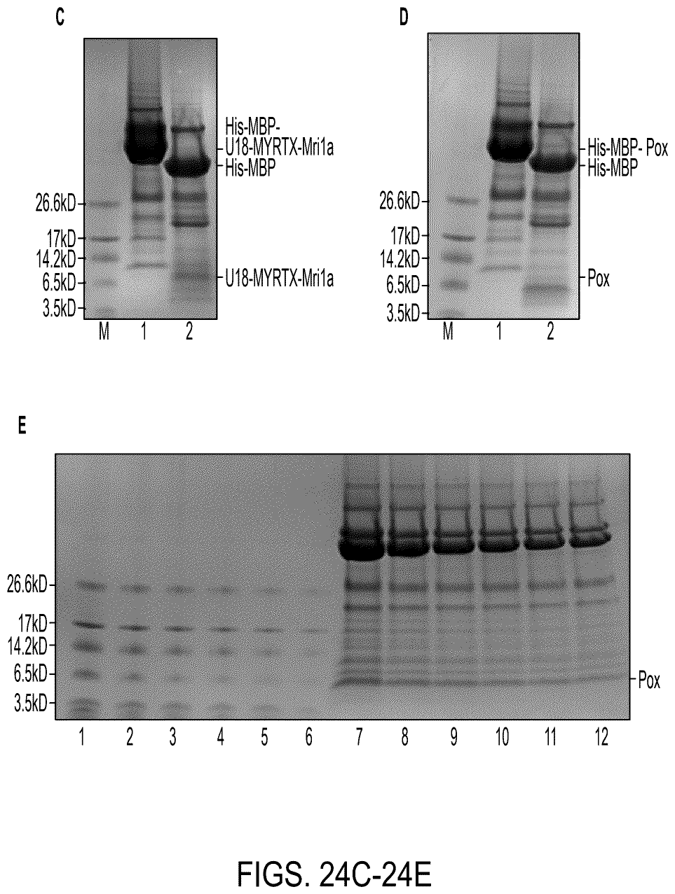

We employed both bead-based (FIG. 8A, iii) and cell-based (FIG. 8A, iv) screening approaches by incubating phage libraries with either magnetic beads coated with receptor extracellular domain-immunoglobulin (ECD-Tg) fusion proteins or cells overexpressing target receptors. Screens were performed in triplicate to assess reproducibility. Candidates were identified by sequencing the collected binders after washing away unbound phage particles (FIG. 8A, v to viii). Known endogenous ligands of EGFR (EGF, BTC, HB-EGF, TGFα Mri1a and Mg1a) were robustly identified using beads coated with Ig-EGFR and HEK293T cells overexpressing EGFR (FIG. 8E). In parallel, six of the seven known endogenous ligands of CXCR2 (CXCR1, 2, 3, 5, 6, 7, 8) were successfully re-discovered after screening against CXCR2-overexpressing HEK293T cells (FIG. 8F). We next established a workflow to reformat screening candidates into purified protein preparations for subsequent bioactivity testing (FIG. 24A, B). EGFR activation studies validated this workflow (FIG. 8G). These results highlight the robustness and utility of our screening platform for identifying biologically relevant ligand-receptor interactions.

### (c) Construction of a Comprehensive Animal Venom Scaffold Library

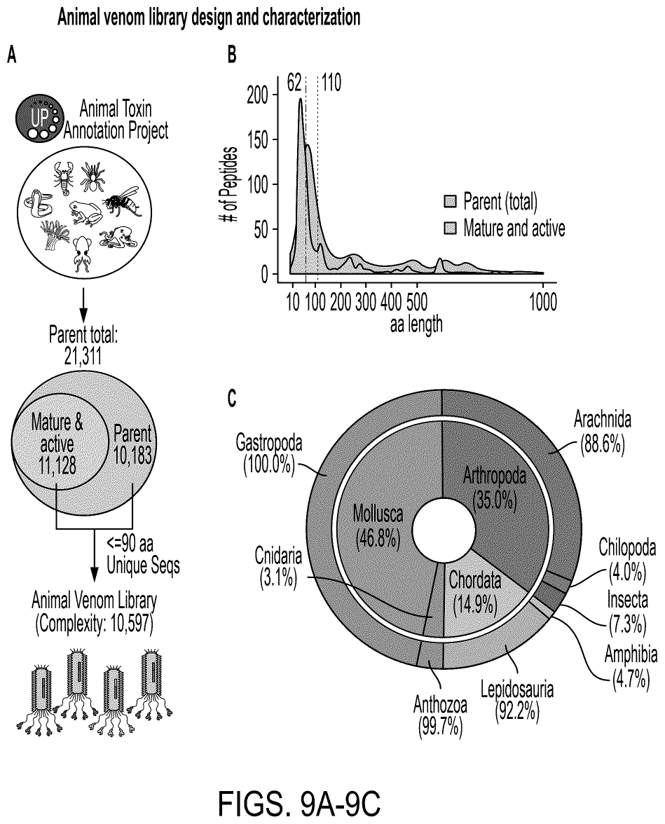

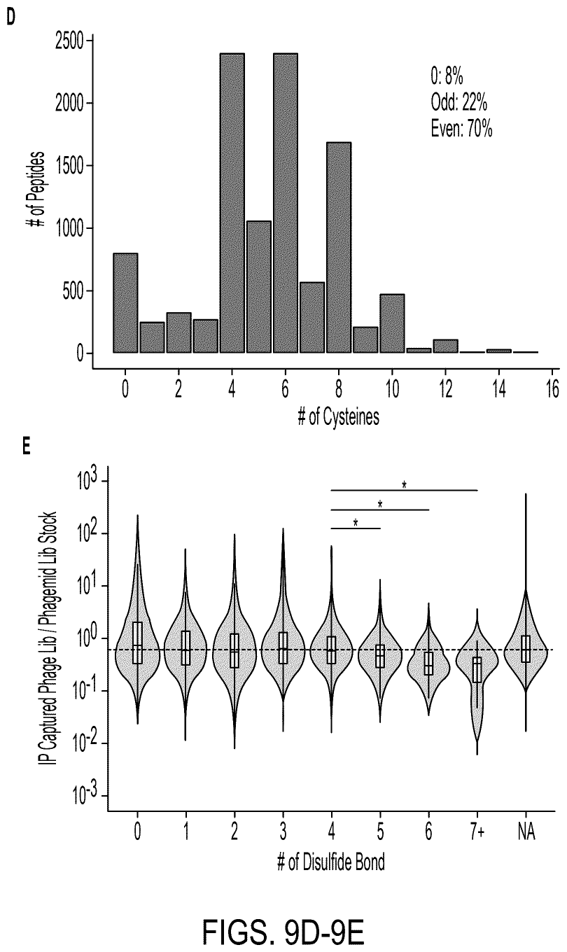

We next designed a diverse library of all known animal venoms, which encompasses highly divergent target classes and bioactivities. A total of 21,311 animal venom (AV) sequences were retrieved from the Uniprot database to serve as a parental set of sequences. By parsing annotations describing post-translational modifications (PTMs) and/or processing events from Uniprot, we were able to extract 11,128 mature and active forms of these sequences. The remaining 10,183 sequences lacked such features. The final AV library included 10,597 mature and active sequences that were less than or equal to 90 amino acids in length (FIG. 9A). A comparison of the length distributions of parental sequences and mature, active sequences revealed a lower median length for the latter, with 62.0% of mature and active sequences having a length of 90 amino acids or less (FIG. 9B), thus being capturable into our AV library design.

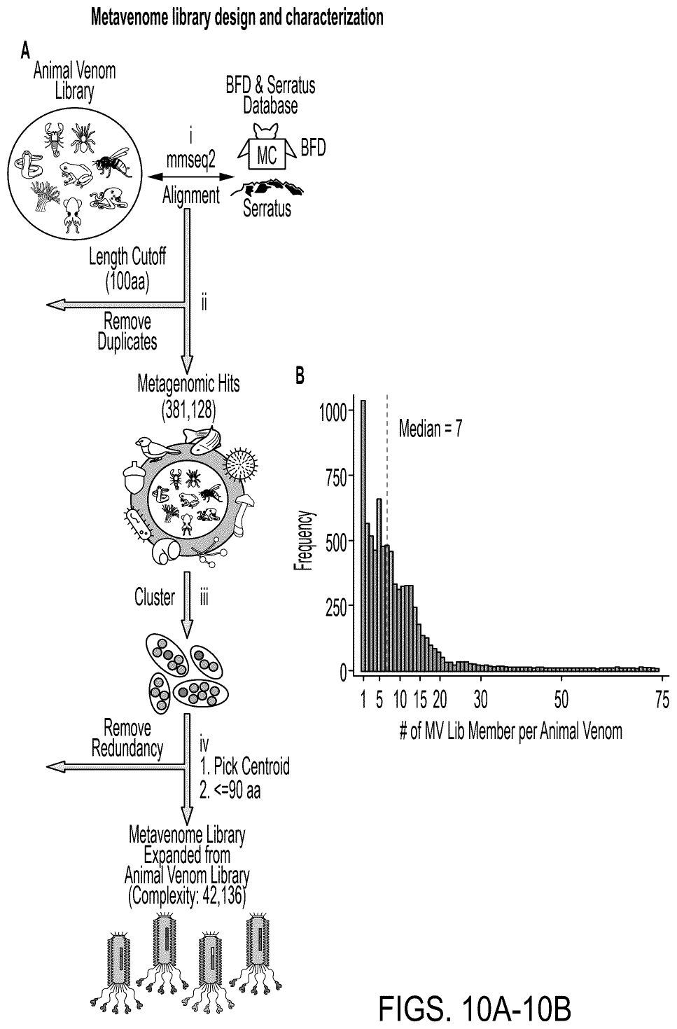

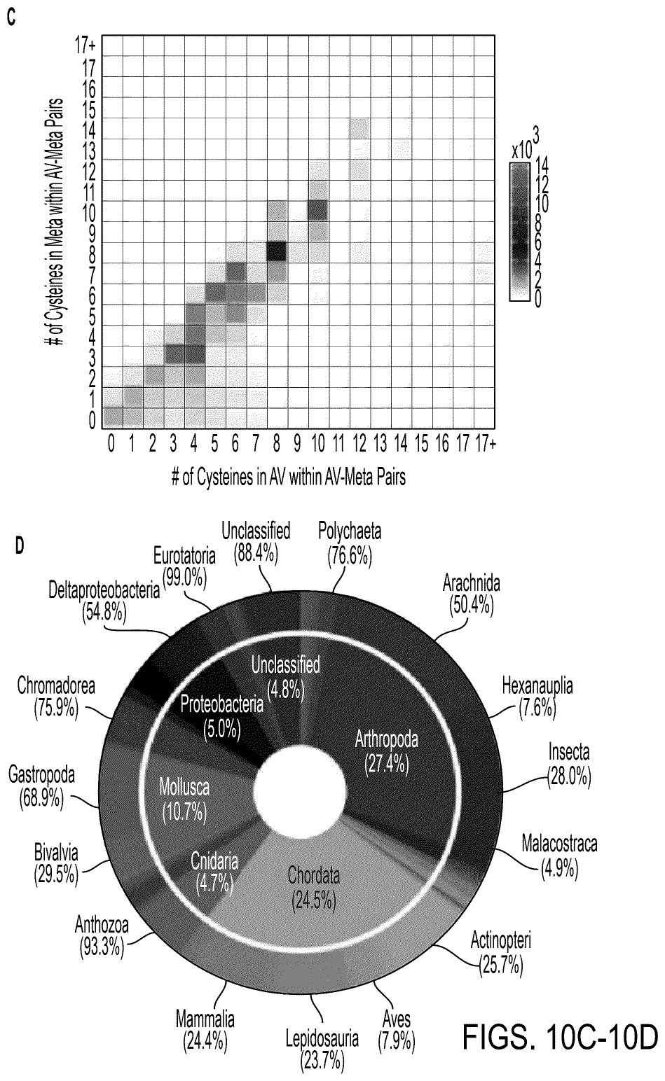

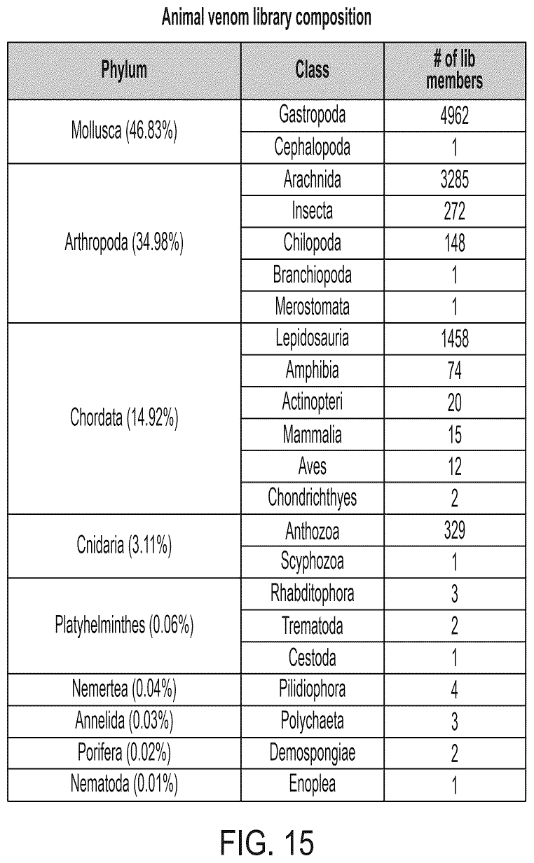

The organisms represented in the final AV library span 22 taxonomic classes, with the majority of sequences originating from cone snails (Gastropoda), scorpions and spiders (Arachnida), and snakes (Lepidosauria) (FIG. 10C, FIG. 15). The library displayed a broad distribution of cysteine numbers: sequences devoid of cysteines make up 8% of the total, those with an odd number of cysteines account for 22%, while a substantial majority, 70%, contain an even number of cysteines. Remarkably, 90.2% sequences contain at least two cysteines, suggesting that disulfide bond formation is likely to be a key feature of this unique and diverse scaffold library (FIG. 10D).

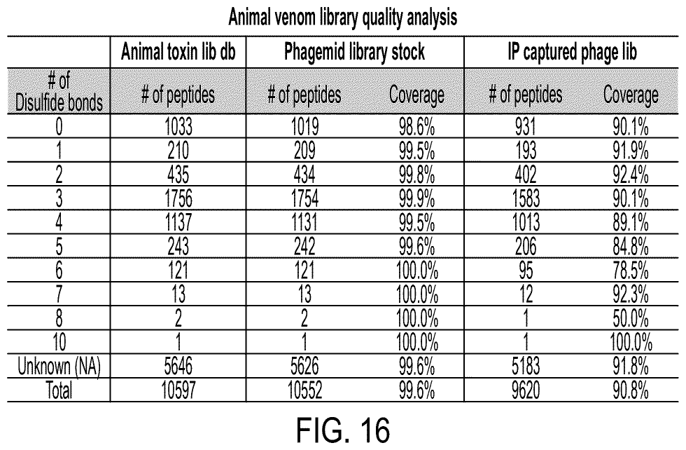

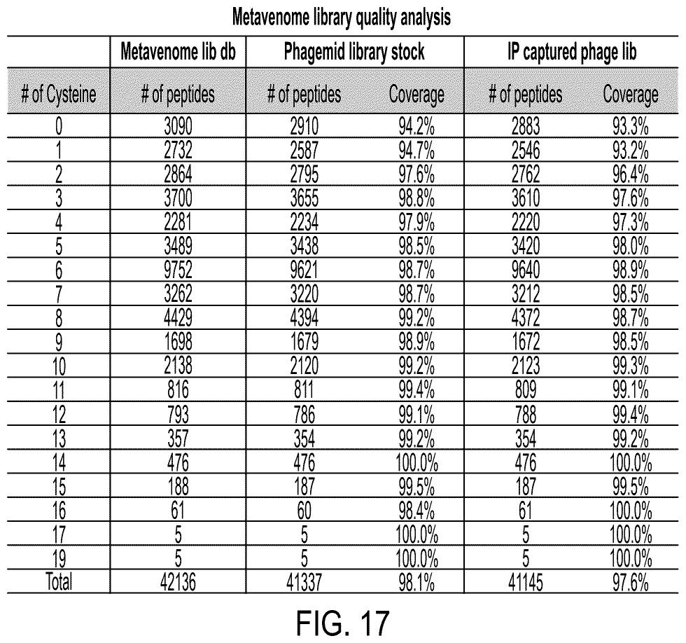

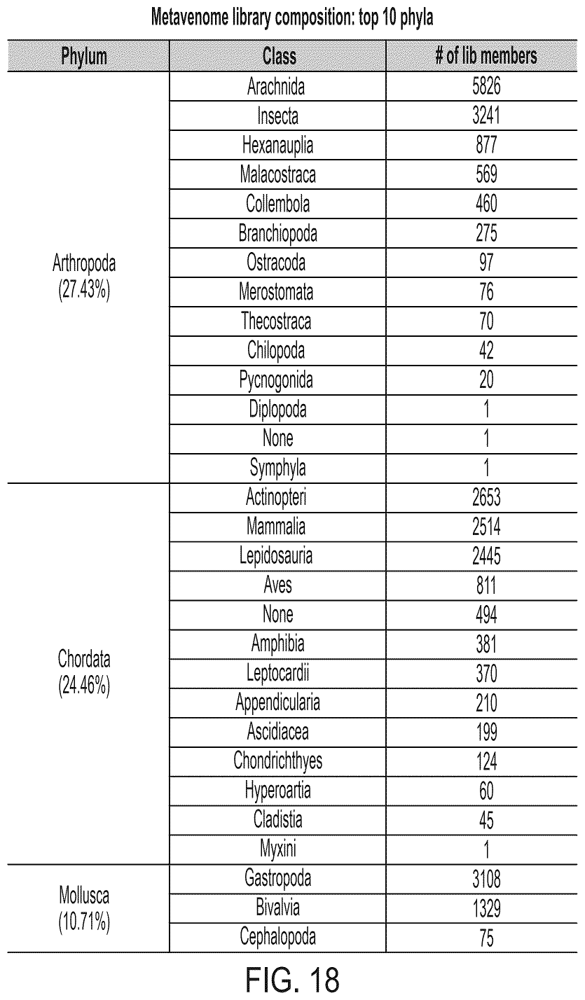

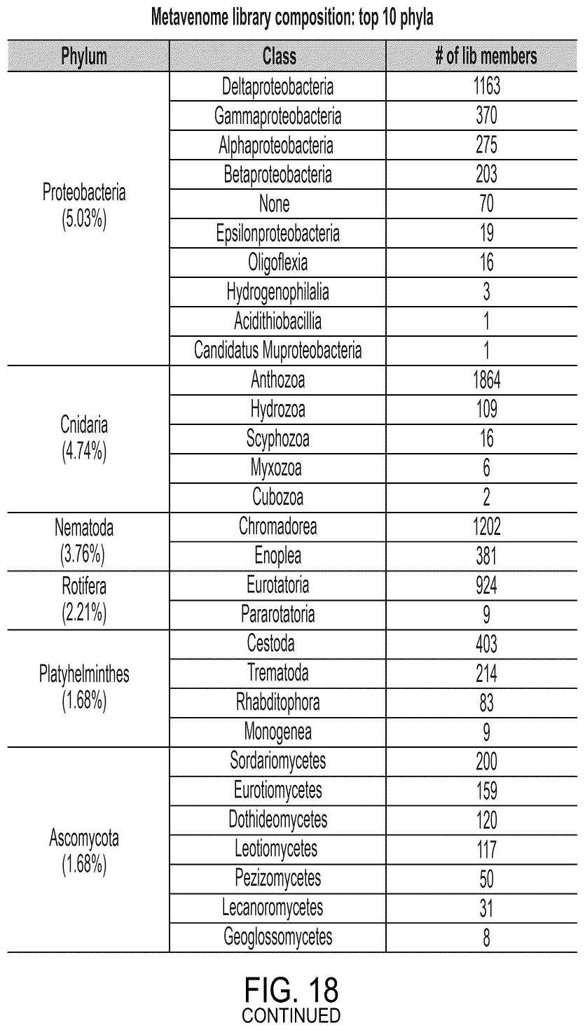

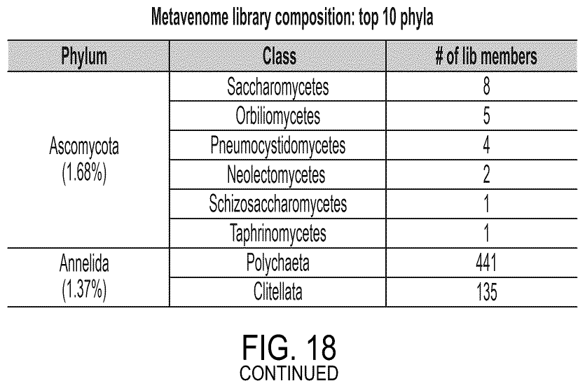

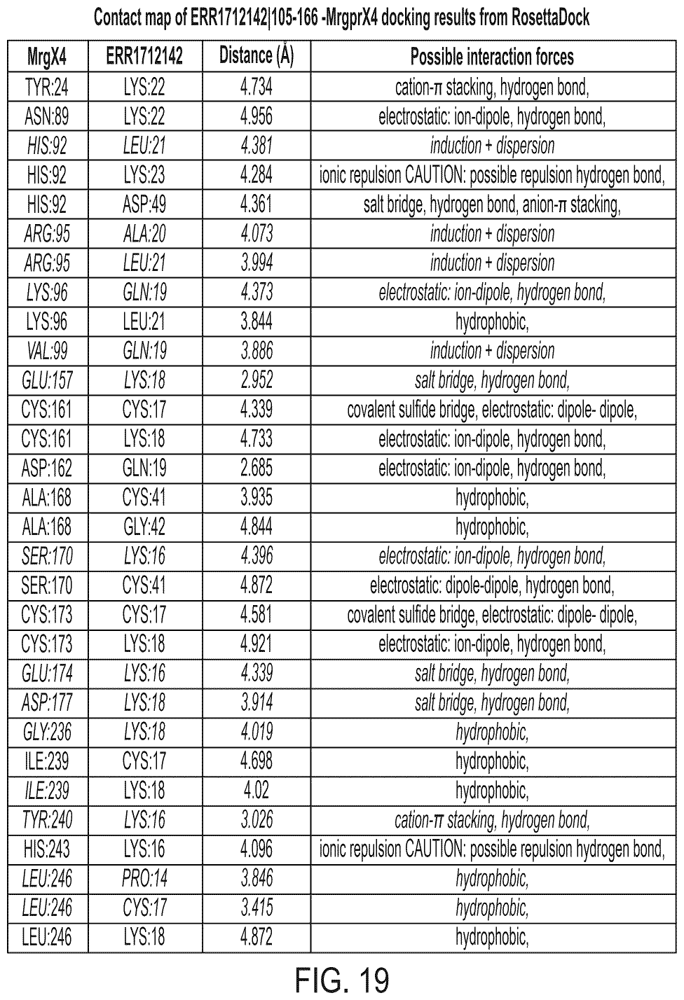

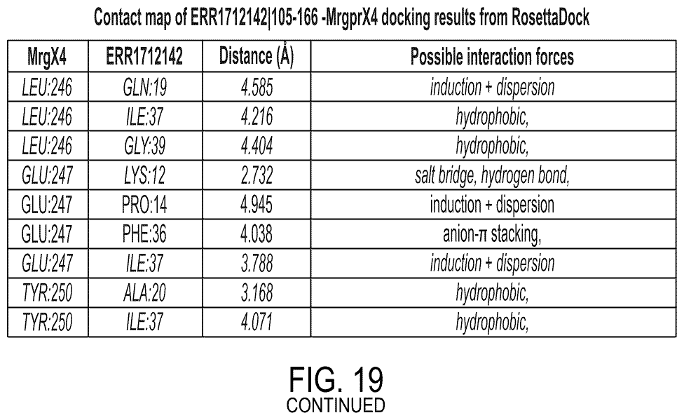

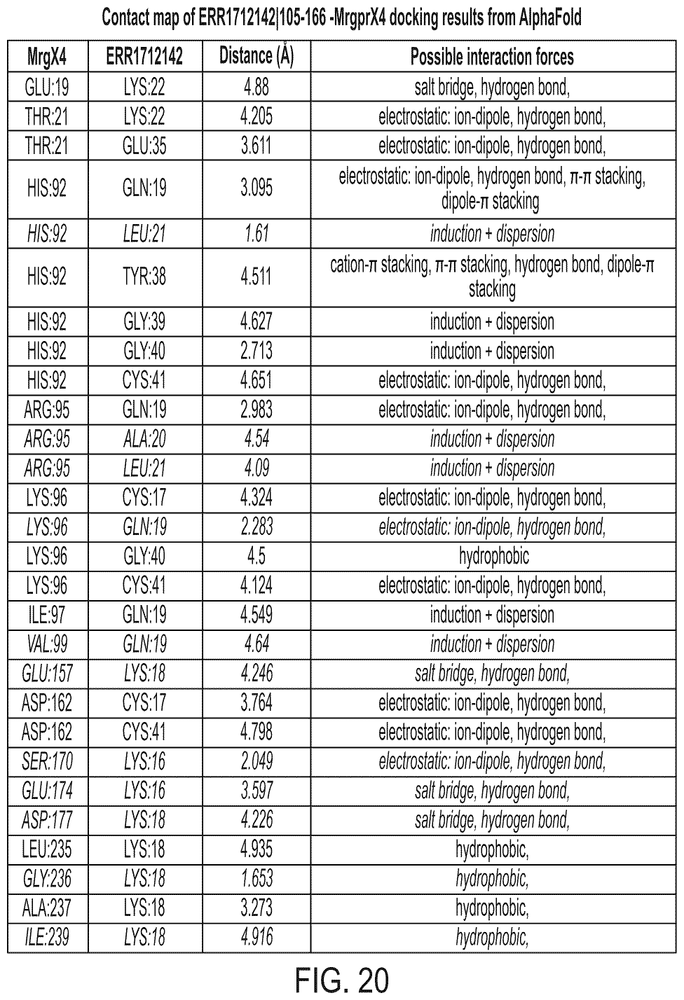

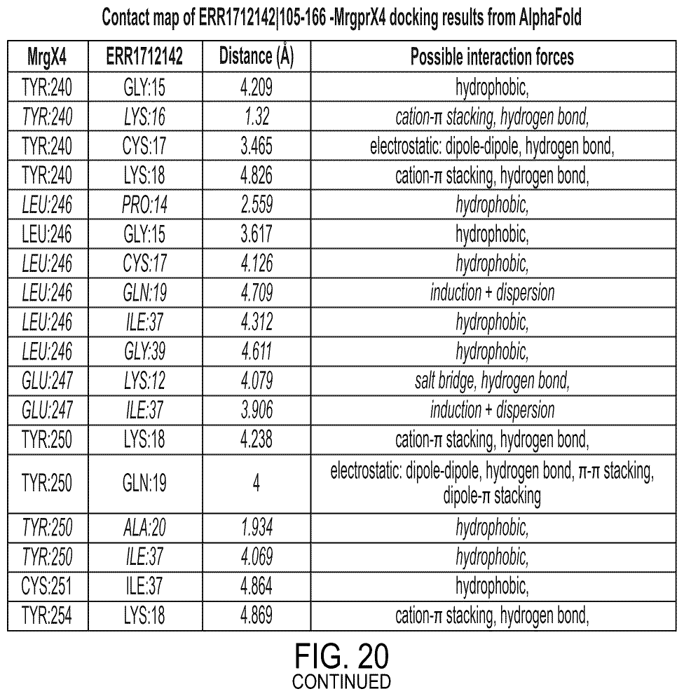

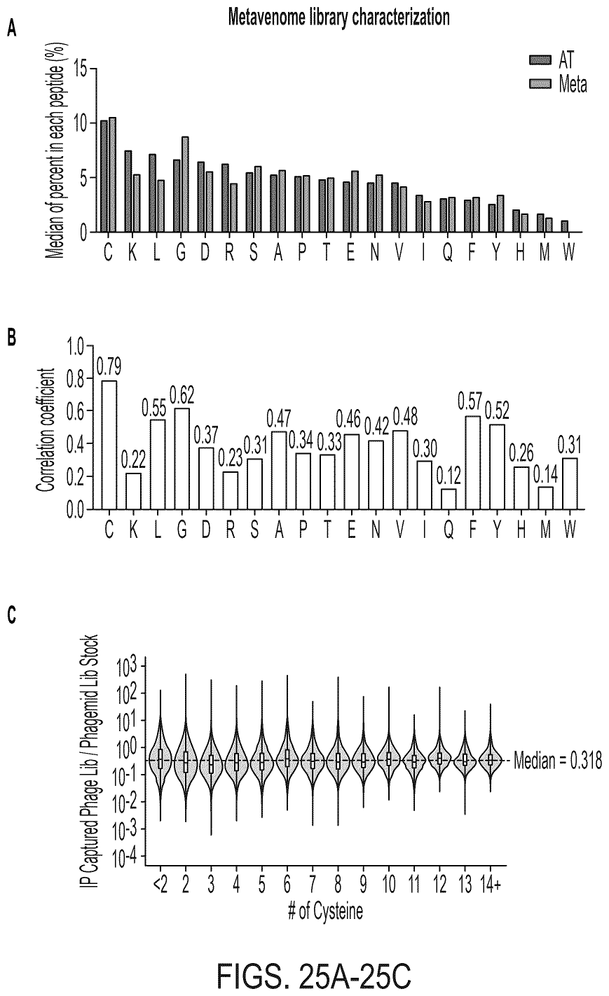

In the cloned library, 99.6% of animal venom library sequences were detectable, with 98.2% of the sequences present within one log (plus or minus) of the median. After phage packaging, 90.8% of the sequences were observed in the final phage library (FIG. 25A, B, FIG. 16). This distribution is in accordance with typical M13 phage display libraries. Factors contributing to skewness include uneven amplification and propagation of different sequences, as well as differential secretion efficiencies of phage particles from the bacterial periplasm. We found no strong correlation between the yield of phage particles and the number of disulfide bonds until four or more were possible, at which point a slight decrease in yields was observed. It is important to note that the exact number of disulfide bonds may differ between the E. coli produced fusion protein and the native polypeptide's annotations in Uniprot (FIG. 9E).

### (d) Expansion of the Animal Venom Library Via Mining for Sequence Homology in Large Metagenomic Databases

To further expand the search space of animal venom related molecular scaffolds, metagenomic databases were mined via high-speed sequence alignment. Using animal venoms as annotated queries in our search for homologous sequences, we employed MMseqs2 to search two databases, the BFD and Serratus (FIG. ′10A, i). After removal of identical target sequences and those longer than 100 amino acids, 381,128 metagenomic sequences with toxin-like attributes were identified. Here we refer to these sequences as the “metavenome” (FIG. 10A, ii). To create a diverse yet compact set of sequences for the library, the metavenome was clustered and sequences filtered out if they exhibited at least 50% sequence identity and 95% overlap with an animal venom sequence (FIG. 10A, iii). DNA oligonucleotides encoding 42,136 cluster representatives equal to or shorter than 90 amino acids were selected to comprise the M13 metavenome (MV) library (FIG. 10A, iv).

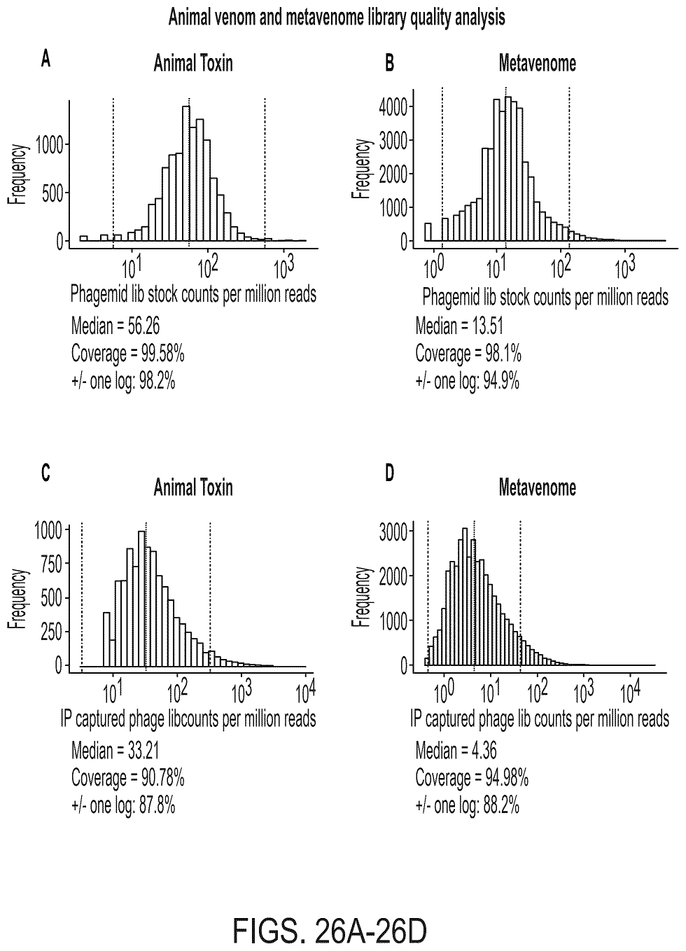

We found a median value of 7 metagenomic hits for each animal venom sequence (FIG. 10B) and importantly, high correlation of cysteine numbers between animal venom and metagenomic pairs (FIG. 10C). Whereas most other amino acids showed little or no correlation, phenylalanine and tyrosine exhibited significant correlation despite being less abundant (FIG. 1g. S4A, B). 98.1% of the MV library members were cloned into the M13-70 phagemid vector, and approximately 95% of library members were successfully represented in the final phage library (FIG. 26C, D). We observed no significant correlation between the yield of the phage particles and the number of cysteines in the protein sequences (FIG. 25C).

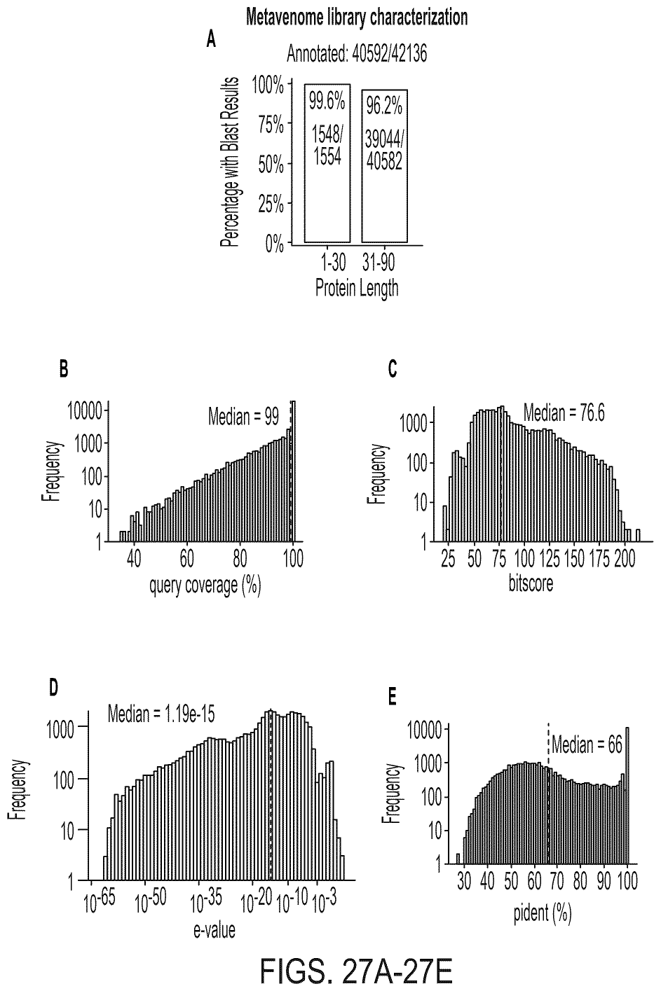

Given that most sequences in the BFD or Serratus databases are not well-annotated or studied, we utilized NCBI-BLAST+ to query the non-redundant database for protein annotations (including taxonomic classifications) that could be adapted to the metavenome library members. 96.3% of MV library members were able to acquire an annotation, with a median evalue of 1.19e−15, suggesting a high level of confidence in the annotated results (FIG. 27D). These metavenome annotations revealed a vastly expanded taxonomic diversity, including proteins from 130 different phyla and 210 different classes (FIG. 10D).

(e) Diverse EGFR Ligands Discovered from the Animal Venom and Metavenome Libraries

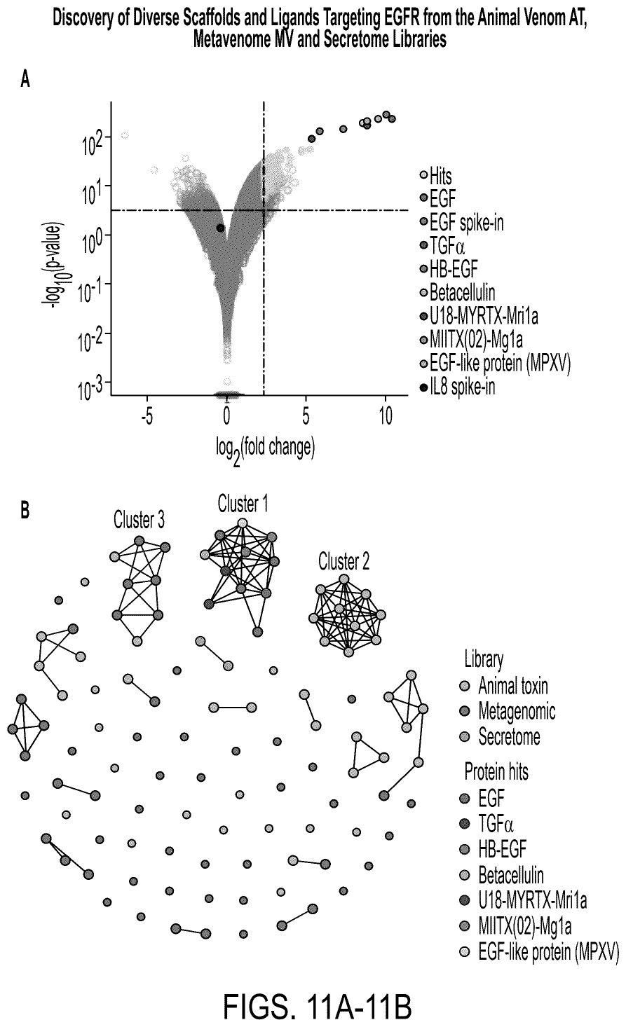

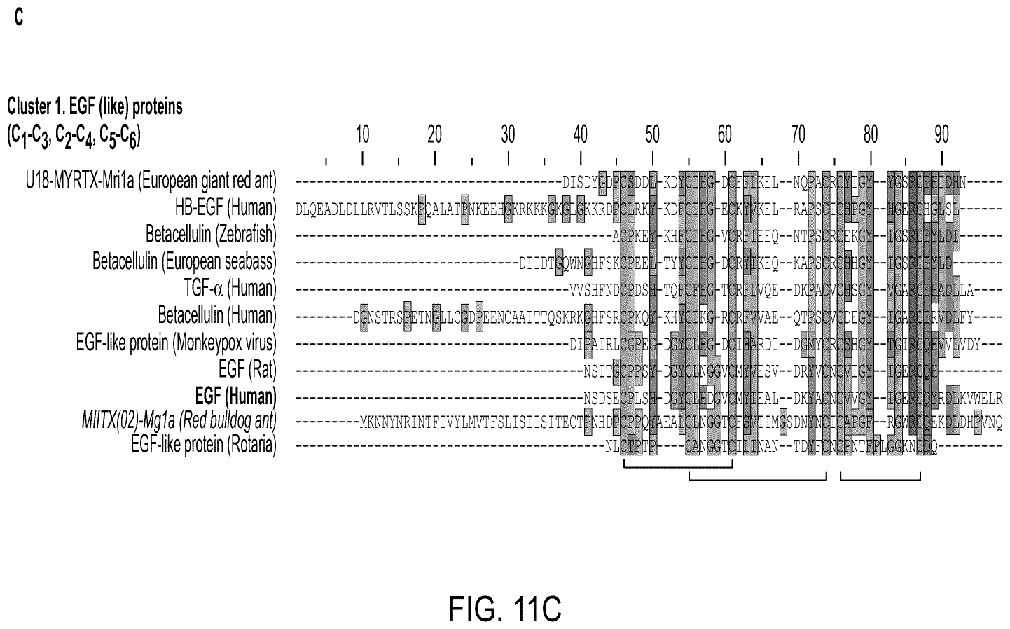

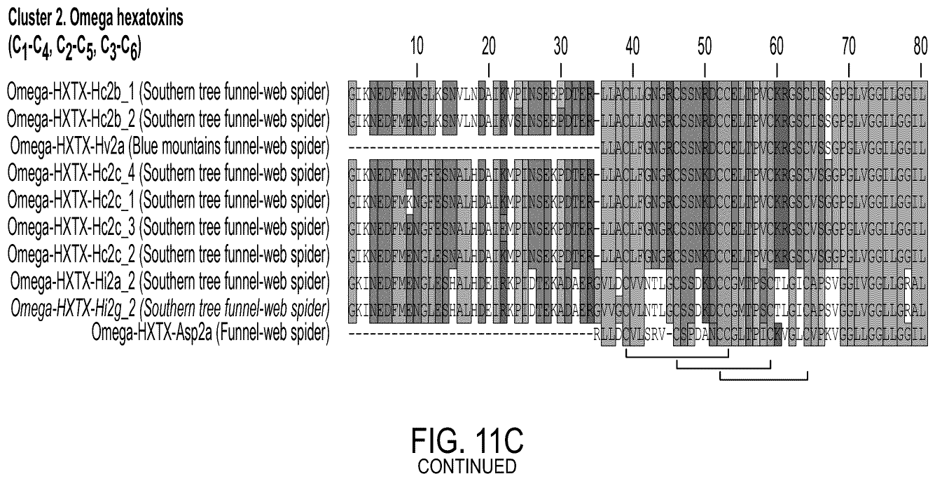

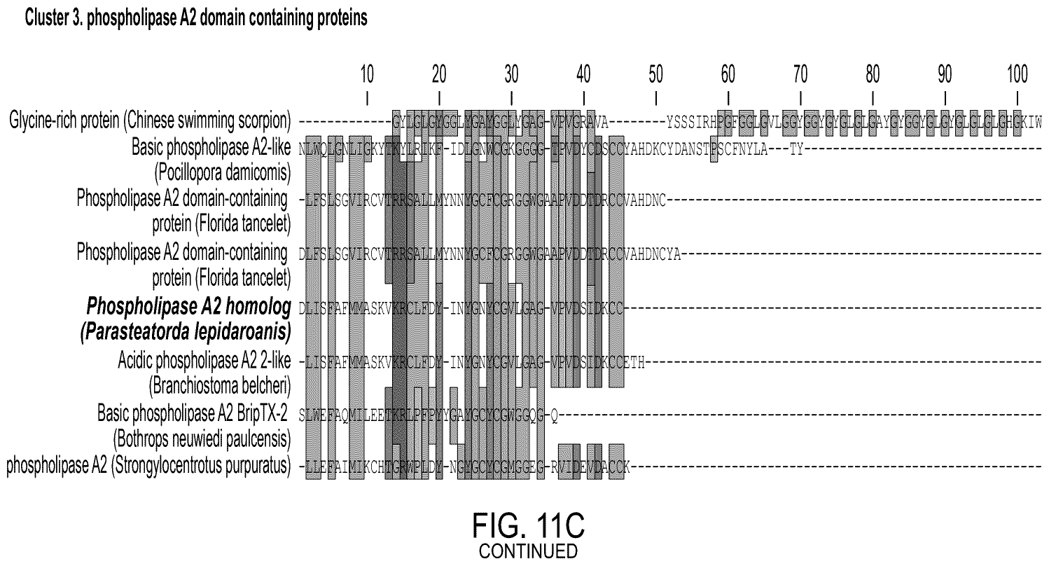

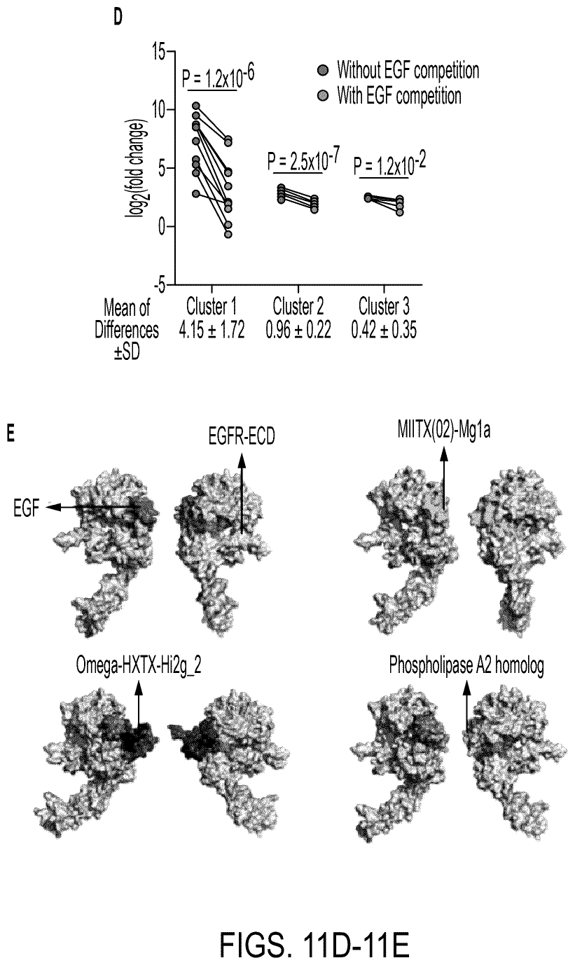

Using EGFR as our model system, we performed a comprehensive screening of the AT, MV, and secretome libraries using Ig-EGFR as the target. Candidate hits were determined by comparing sequencing results against a negative control immunoglobulin (Isotype Ig) from triplicate screens. We identified a multitude of candidate binders from all three libraries, in addition to the endogenous ligands (EGF, BTC, TGFa and HB-EGF) and previously reported animal venoms (Mg1a and Mri 1a) (FIG. 11A).

We employed a sequence-based network graph approach to represent the sequence similarity among all candidate EGFR ligands. In this approach, candidate EGFR ligands, represented as nodes, were linked if they shared sequence similarity as determined by protein sequence alignment. Although many clones demonstrated no sequence homology to other hits, three dominant clusters emerged (FIG. 11B). To identify regions of sequence similarity that could underlie binding, multiple sequence alignments (MSAs) were next generated via Clustal Omega (FIG. 11C). Among all members of cluster 1, which includes human EGF, we observed absolute conservation of the cysteine amino acids known to be crucial for EGF binding. Cluster 2 was composed exclusively of sequences from spider omega hexatoxins of the animal venom library. Cluster 2 also exhibited absolute conservation of cysteine residues, but with a disulfide bond configuration distinct from EGF. Sequences in Cluster 3 did not appear to possess disulfide bonds, and instead appeared to feature a shared phospholipase A2 domain.

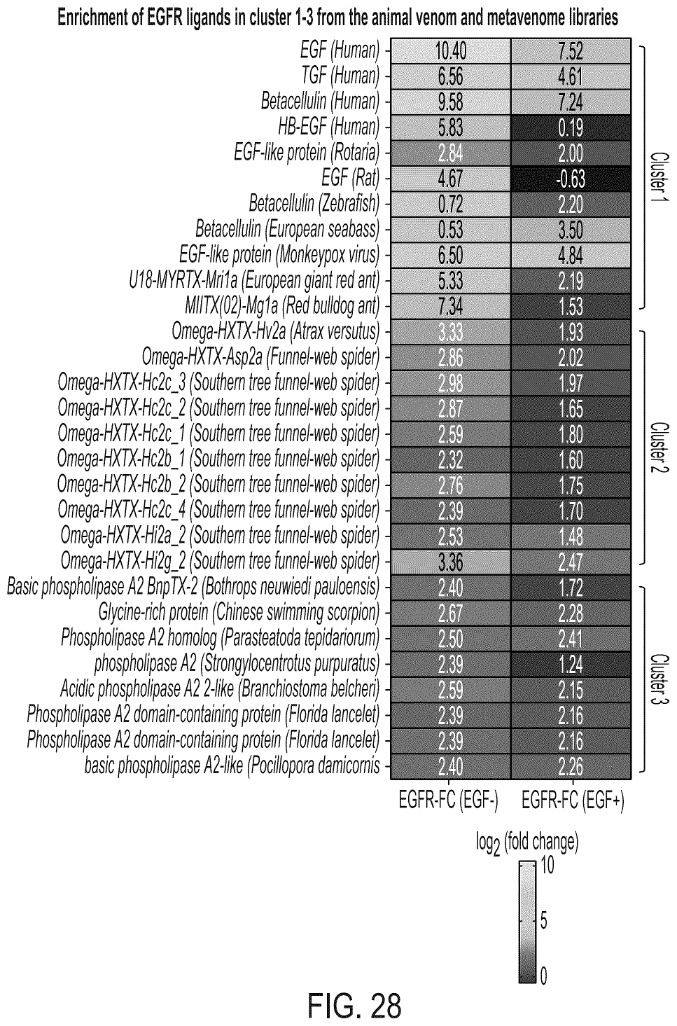

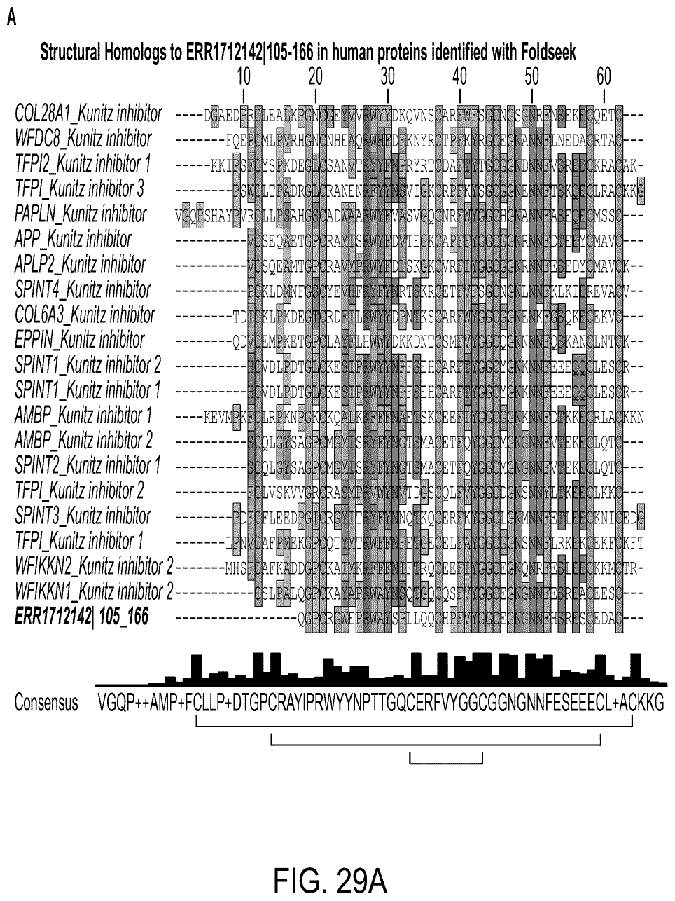

Conserved sequence motifs among candidate hits suggest a shared binding mode. Clusters 2 and 3 exhibited significantly lower binding strength to EGFR versus Cluster 1. In the presence of competing EGF, the binding strengths of ligands in Cluster 1 were greatly diminished, indicative of competitive binding to the receptor (FIG. 28, FIG. 11D). In contrast, EGF competition only minimally impacted binding of proteins in Clusters 2 and 3, suggestive of an alternative binding mode (FIG. 11D). To investigate potential binding modes, a representative from each cluster underwent RosettaDock modeling with EGFR (FIG. 11E). Ant venom (MIITX(02)-Mg1a), a known activator of EGFR signaling, was selected from Cluster 1 and accurately docked into the ligand-receptor binding pocket. However, Omega-HXTX-Hi2g_2 (Cluster 2) and Phospholipase A2 homolog (Cluster 3) docked outside of, but slightly overlapped with the ligand-receptor binding pocket (FIG. 11E), consistent with insensitivity to EGF competition. Engineered proteins derived from these lower-affinity binders may therefore yield novel candidate EGFR antagonists with clinical relevance.

### (f) Discovery of a Kunitz Type Domain Containing Protein as a Potential Endogenous Antagonist of MrgprX4

The MrgprX4 receptor plays a crucial role in the perception of pain and itch sensations. Recent studies have illuminated that MrgprX4 is activated by certain bile acids, such as Ursodeoxycholic acid (UDCA). This activation triggers calcium dependent neuronal excitation, culminating in the sensations of pain and itch. Activation of this pathway is believed to be a key feature of liver disease-associated cholestatic pruritus^{29,30}. Inhibition of MrgprX4 activation is therefore a promising therapeutic avenue to address a substantial unmet clinical need.

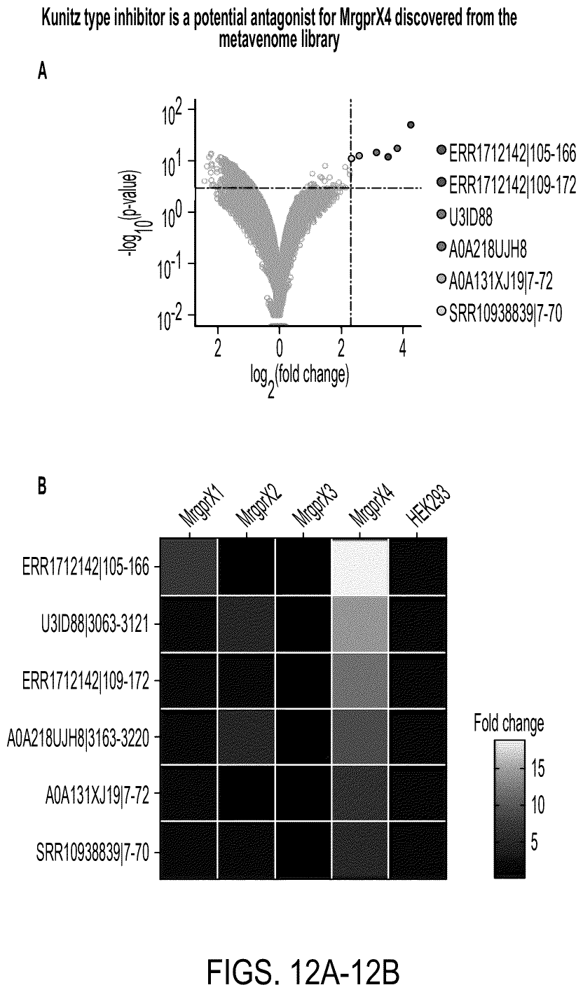

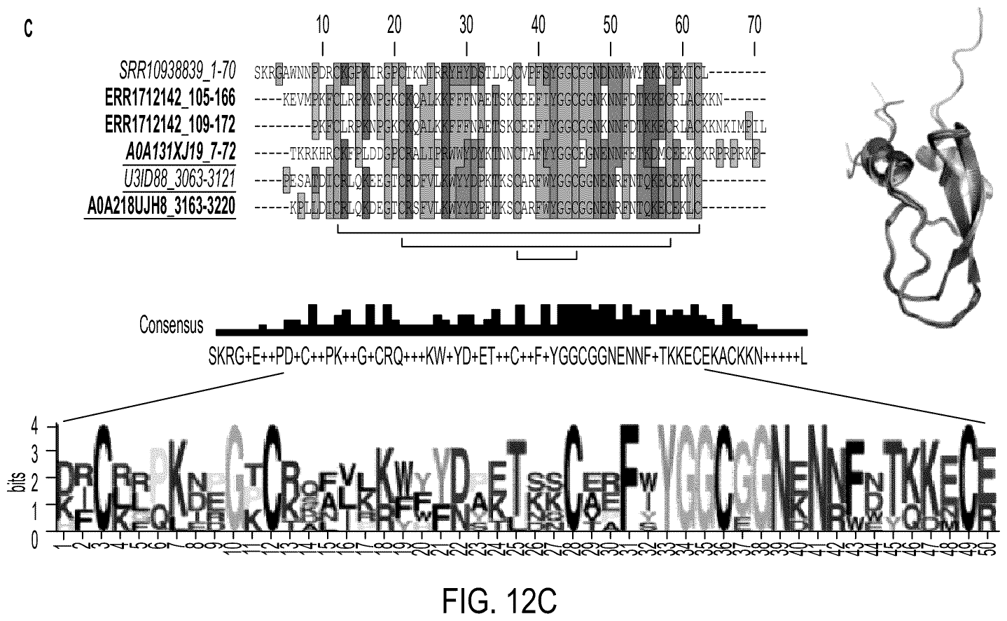

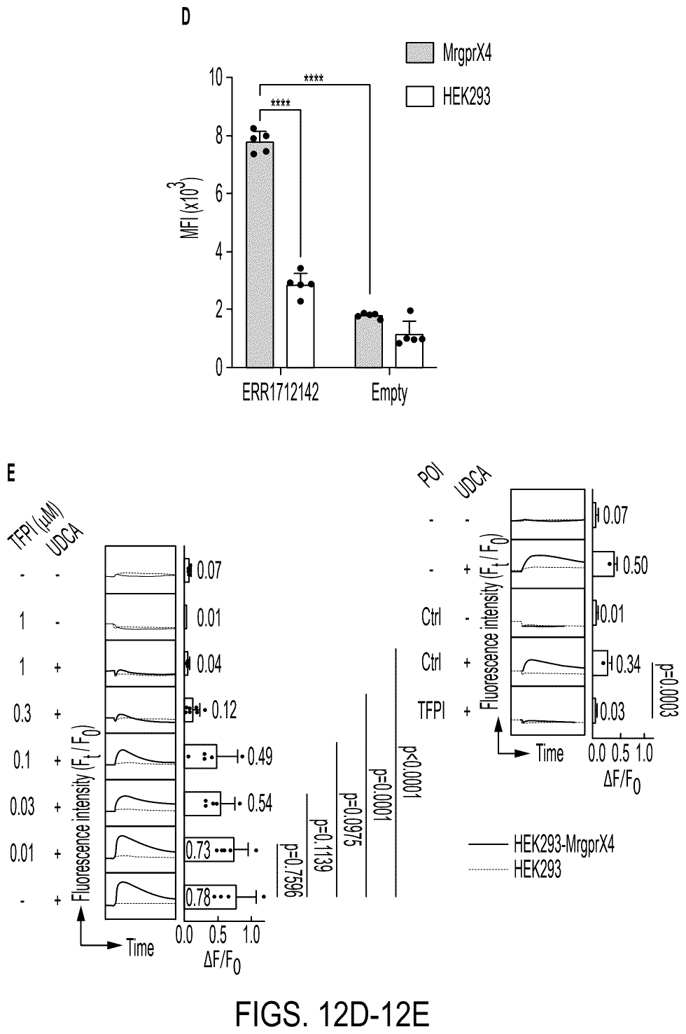

We conducted a screening campaign using our animal venom, metavenome, and secretome libraries against cells overexpressing MrgprX4 (HEK-MrgprX4) and related members of the MrgprX family (MrgprX1, MrgprX2, and MrgprX3) (FIG. 12A-B). We discovered six proteins from the metavenome library that demonstrated selective binding to MrgprX4 (FIG. 12B). These proteins contain a highly conserved motif, identified via multiple sequence alignment (FIG. 12C). High structural homology among these six proteins is also predicted by AlphaFold (FIG. 12C).

To confirm binding of putative MrgprX4 ligands, we produced the candidate hit with the highest fold-change value—ERR1712142|105-166—and evaluated binding to both MrgprX4 overexpressing cells and parent HEK293 cells. ERR1712142|105-166 had significantly higher binding to the MrgprX4 overexpressing cells compared to that of HEK293 cells (FIG. 12D).

In a search for endogenous homologues of these candidate ligands, we used Foldseek”, to find human structural homologs of ERR1712142|105-166. The databases interrogated included AlphaFoldDB (version 4: Proteomes and Swiss-Prot), AlphaFoldDB (version 4), CATH clustered at 50% sequence identity, ESM Atlas-HQ, and the Protein Data Bank (PDB). All 20 of the unique human protein sequences identified as structural homologs to ERR1712142|105-166 are members of the Kunitz-type protease inhibitor class (FIG. 29). Kunitz-type protease inhibitor class proteins contain a common domain, the Kunitz-type inhibitor domain, which is characterized by six cysteines forming a distinctive disulfide bond pattern of C1-C6, C2-C5, C3-C4. Kunitz-type inhibitor domain containing proteins are therefore candidate MrgprX4 ligands that that may have agonist or antagonist activities on MrgprX4 signaling.

Among the human Kunitz-type inhibitor domain containing proteins, tissue factor pathway inhibitor (TFPI) possesses three such domains that align with ERR1712142|105-166 (e-values lower than 10⁻³). Utilizing a calcium-signaling assay, we determined that TFPI does not activate MrgprX4 at concentrations up to 1 M. In contrast, TFPI effectively inhibited UDCA-induced activation of MrgprX4 at concentrations down to 0.3 μM. A negative control protein, osteoprotegerin (OPG), manufactured identically to the TFPI, had no obvious inhibitory activity on MrgprX4.

### References Example 4

1. Peigneur, S. & Tytgat, J. Toxins in drug discovery and pharmacology. Toxins (Basel). 10, 10-13 (2018).
2. Harvey, A. L. Toxins and drug discovery. Toxicon 92, 193-200 (2014).
3. Lewis, R. & Garcia, M. Therapeutic potential of venom peptides. Nat Rev Drug Discov 2, (2003).
4. Smallwood, T. B. & Clark, R. J. Advances in venom peptide drug discovery: where are we at and where are we heading? Expert Opin. Drug Discov. 16, 1163-1173 (2021).
5. Robinson, S. D., Undheim, E. A. B., Ueberheide, B. & King, G. F. Venom peptides as therapeutics: advances, challenges and the future of venom-peptide discovery. Expert Rev. Proteomics 14, 931-939 (2017).
6. Muttenthaler, M., King, G. F., Adams, D. J. & Alewood, P. F. Trends in peptide drug discovery. Nat. Rev. DrugDiscov. 2021 204 20, 309-325 (2021).
7. Nauck, M. A. & D'Alessio, D. A. Tirzepatide, a dual GIP/GLP-1 receptor co-agonist for the treatment of type 2 diabetes with unmatched effectiveness regrading glycaemic control and body weight reduction. Cardiovasc. Diabetol. 21, 1-16 (2022).
8. Frias, J. P. et al. Tirzepatide versus Semaglutide Once Weekly in Patients with Type 2 Diabetes. N. Engl. J Med. 385, 503-515 (2021).
9. King, G. F. Venoms as a platform for human drugs: translating toxins into therapeutics. https://doi.org 10.1517/14712598.2011.621940 11, 1469-1484 (2011).
10. Vetter, I. et al. Venomics: a new paradigm for natural products-based drug discovery. Amin. Acids 2010 401 40, 15-28 (2010).
11. Hauser, A. S., Attwood, M. M., Rask-Andersen, M., Schiöth, H. B. & Gloriam, D. E. Trends in GPCR drug discovery: New agents, targets and indications. Nat. Rev. Drug Discov. 16, 829-842 (2017).
12. Yin, H. & Flynn, A. D. Drugging Membrane Protein Interactions. https:1doi.org 10.1146 annurev-bioeng-092115-025322 18, 51-76 (2016).
13. Kelil, A., Gallo, E., Banerjee, S., Adams, J. J. & Sidhu, S. S. CellectSeq: In silico discovery of antibodies targeting integral membrane proteins combining in situ selections and next-generation sequencing. Commun. Biol. 4, 1-13 (2021).
14. Lavergne, V., J. Taft, R. & F. Alewood, P. Cysteine-Rich Mini-Proteins in Human Biology. Curr. Top. Med. Chem. 12, 1514-1533 (2012).
15. Correnti, C. E. et al. Screening, large-scale production and structure-based classification of cystine-dense peptides. Nat. Struct. Mol. Biol. (2018) doi:10.1038/s41594-018-0033-9.
16. Crook, Z. R., Nairn, N. W. & Olson, J. M. Miniproteins as a Powerful Modality in Drug Development. Trends Biochem. Sci. 45, 332-346 (2020).
17. Vazquez-Lombardi, R. et al. Challenges and opportunities for non-antibody scaffold drugs. Drug Discov. Today 20, 1271-1283 (2015).
18. Lobba, A. R. M. et al. A Kunitz-type inhibitor from tick salivary glands: A promising novel antitumor drug candidate. Front. Mol. Biosci. 9, 936107 (2022).
19. Simeon, R. & Chen, Z. In vitro-engineered non-antibody protein therapeutics. Protein Cell 9, 3-14 (2018).
20. Lewis, R. J. & Garcia, M. L. Therapeutic potential of venom peptides. Nat. Rev. Drug Discov. 2003 210 2, 790-802 (2003).
21. Jungo, F., Bougueleret, L., Xenarios, I. & Poux, S. The UniProtKB/Swiss-Prot Tox-Prot program: A central hub of integrated venom protein data. Toxicon 60, 551-557 (2012).
22. Bateman, A. et al. UniProt: A hub for protein information. Nucleic Acids Res. (2015) doi:10.1093/nar/gku989.
23. Jumper, J. et al. Highly accurate protein structure prediction with AlphaFold. Nat. 2021 5967873 596, 583-589 (2021).
24. Edgar, R. C. et al. Petabase-scale sequence alignment catalyses viral discovery. Nat. 2022 6027895 602, 142-147 (2022).
25. Rondot, S., Koch, J., Breitling, F. & Dubel, S. A helper phage to improve single-chain antibody presentation in phage display. Nat. Biotechnol. 19, 75-78 (2001).
26. Gabibov, A. G. & Belogurov, A. A. Probing Surface Membrane Receptors Using Engineered Bacteriophage Bioconjugates. (2019) doi:10.1021/acs.bioconjchem.9b00218.
27. Frenzel, A., Schirrmann, T. & Hust, M. Phage display-derived human antibodies in clinical development and therapy. MAbs 8, 1177 (2016).
28. Sanders, J. M., Wampole, M. E., Thakur, M. L. & Wickstrom, E. Molecular Determinants of Epidermal Growth Factor Binding: A Molecular Dynamics Study. PLoS One 8, e54136 (2013).
29. Meixiong, J., Vasavda, C., Snyder, S. H. & Dong, X. MRGPRX4 is a G protein-coupled receptor activated by bile acids that may contribute to cholestatic pruritus. Proc. Natl. Acad. Sci. U.S.A 116, 10525-10530 (2019).
30. Yu, H. et al. MRGPRX4 is a bile acid receptor for human cholestatic itch. Elife 8, (2019).
31. van Kempen, M. et al. Fast and accurate protein structure search with Foldseek. Nat. Biotechnol. 2023 1-4 (2023) doi:10.1038/s41587-023-01773-0.
32. Wang, J. et al. Scaffolding protein functional sites using deep learning. Science (80-.). 377, 387-394 (2022).
33. Dauparas, J. et al. Robust deep learning based protein sequence design using ProteinMPNN. doi: 10.1101/2022.06.03.494563.
34. Cao, L. et al. De novo design of picomolar SARS-CoV-2 miniprotein inhibitors. Science (80-.). 370, 426-431 (2020).
35. Credle, J. J. et al. Unbiased discovery of autoantibodies associated with severe COVID-19 via genome-scale self-assembled DNA-barcoded protein libraries. Nat. Biomed. Eng. 2022 68 6, 992-1003 (2022).
36. Zhu, J. et al. Protein interaction discovery using parallel analysis of translated ORFs (PLATO). Nat. Biotechnol. 2013 314 31, 331-334 (2013).
37. Lown, P. S. et al. Extended yeast surface display linkers enhance the enrichment of ligands in direct mammalian cell selections. Protein Eng. Des. Sel. 34, 1-9 (2021).
38. Lown, P. S. & Hackel, B. J. Magnetic Bead-Immobilized Mammalian Cells Are Effective Targets to Enrich Ligand-Displaying Yeast. ACS Comb. Sci. 22, 274-284 (2020).
39. Csizmar, C. M. et al. Multivalent Ligand Binding to Cell Membrane Antigens: Defining the Interplay of Affinity, Valency, and Expression Density. J Am. Chem. Soc. 141, 251-261 (2019).
40. Shen, W. & Ren, H. TaxonKit: A practical and efficient NCBI taxonomy toolkit. J Genet. Genomics 48, 844-850 (2021).
41. Robinson, M. D., McCarthy, D. J. & Smyth, G. K. edgeR: a Bioconductor package for differential expression analysis of digital gene expression data. Bioinformatics 26, 139-140 (2010).
42. McCarthy, D. J., Chen, Y. & Smyth, G. K. Differential expression analysis of multifactor RNA-Seq experiments with respect to biological variation. Nucleic Acids Res. 40, 4288-4297 (2012).
43. Klint, J. K. et al. Production of Recombinant Disulfide-Rich Venom Peptides for Structural and Functional Analysis via Expression in the Periplasm of E. coli. PLoS One 8, e63865 (2013).
44. Badaczewska-Dawid, A. E., Nithin, C., Wroblewski, K., Kurcinski, M. & Kmiecik, S. MAPIYA contact map server for identification and visualization of molecular interactions in proteins and biological complexes. Nucleic Acids Res. 50, W474-W482 (2022).

1. A method of identifying candidate bioactive compounds comprising:
preparing and cloning an oligonucleotide library encoding diverse toxin-like polypeptides from a multitude of scaffolds to create a polypeptide display library;
contacting the toxin-like polypeptide display library with target molecules;
removing members of the toxin-like polypeptide display library that do not sufficiently bind to the target molecules;
sequencing the members of the toxin-like polypeptide display library which bind to the target; and
identifying candidate bioactive compounds.

2. The method of claim 1, wherein the polypeptide display library is a mRNA display library, a ribosome display library, a PLATO library, a MIPSA library, a PepSeq library, a bacteriophage display library, a hyperphage display library, a bacterial display library, a yeast display library, an insect cell display library, an invertebrate cell display library or a mammalian cell display library.

3. The method of claim 2, wherein the bacteriophage display or hyperphage display library comprises an M13 bacteriophage display library or an M13 hyperphage display library.

4. The method of claim 3, wherein the toxin-like polypeptide is fused to the N-terminus of the p3 protein.

5-9. (canceled)

10. The method of claim 1, wherein the displayed toxin-like polypeptides are designed to contain between 2 and 20 cysteine residues, which are capable of forming disulfide bonds.

11-12. (canceled)

13. The method of claim 1, wherein the oligonucleotide library encoding diverse toxin-like polypeptides is mutagenized or undergoes error prone PCR in order to introduce library diversity.

14. The method of claim 1, wherein the number of rounds of target binding is one.

15. The method of claim 1, wherein the number of rounds of target binding is greater than one.

16. The method of claim 15, wherein the toxin-like polypeptide display library is mutagenized between rounds of target binding.

17. The method of claim 16, wherein the toxin-like polypeptide display library is sequenced after and/or between rounds of target binding.

18. The method of claim 1, wherein the target molecules are purified and conjugated to a surface during or after interaction with the toxin-like polypeptide display library.

19. The method of claim 1, wherein the target molecules are expressed in or on target cells.

20. The method of claim 1, wherein the library is sequenced using high throughput DNA sequencing.

21. The method of claim 1, wherein sequencing of the library is used to identify differential binding of toxin-like polypeptide library members to target molecules versus molecules or surfaces lacking the target or to different molecules that are related but not identical to the target molecules, or versus target molecules in the presence of a competing target-binding molecule.

22. The method of claim 1, wherein the oligonucleotide library encoding diverse toxin-like polypeptides is comprised of shuffled domains.

23. The method of claim 1, wherein the displayed toxin-like polypeptides are designed to also display library members that lack a specific or multiple specific cysteine residues to identify disulfide binds that are critical for binding to the target molecules.

24. The method of claim 1, wherein the displayed toxin-like polypeptides comprise versions of candidate bioactive compounds with targeted mutations, such as scanning mutagenesis, or tiled fragments, for the purpose of identifying key residues or regions that are critical for binding to the target molecules.

25-29. (canceled)

30. A method of identifying candidate therapeutic compounds comprising:
preparing and cloning an oligonucleotide library encoding candidate therapeutic polypeptides into a hyperphage phagemid and culturing in bacteria;
infecting the bacteria with a helper phage lacking a native P3 protein and generating a phage library;
contacting the phage library with a target protein or cells expressing a target protein;
sequencing of the phage library bound to the ligands or target proteins; and
identifying candidate therapeutic compounds.

31-37. (canceled)

38. A method for treating a mas-related G protein coupled receptor-mediated condition in a subject, comprising administering an effective amount of a compound comprising a Kunitz-type domain to the subject, thereby treating the G protein coupled receptor-mediated condition.

39. The method of claim 38 wherein the mas-related G protein coupled receptor-mediated condition is an adverse drug reaction, pruritus or other chronic itch condition or an autoimmune disease.

40-53. (canceled)


SEQUENCE LISTING (84 sequences)

SEQ ID NO: 1 - protein, 5 aa, synthetic construct
GGGGS

SEQ ID NO: 2 - protein, 6 aa, synthetic construct
HHHHHH

SEQ ID NO: 3 - DNA, 15 bp, synthetic construct
  source 1..15: mol_type = other DNA
ggaattccgctgcgt

SEQ ID NO: 4 - DNA, 15 bp, synthetic construct
  source 1..15: mol_type = other DNA
ccgagcattggcacc

SEQ ID NO: 5 - DNA, 15 bp, synthetic construct
  source 1..15: mol_type = other DNA
gtcgtgccagggaac

SEQ ID NO: 6 - DNA, 15 bp, synthetic construct
  source 1..15: mol_type = other DNA
gcctggagacgccac

SEQ ID NO: 7 - protein, 11 aa, unidentified
MQVIRILVTGA

SEQ ID NO: 8 - protein, 12 aa, synthetic construct
MAEPIRVIVTGI

SEQ ID NO: 9 - protein, 86 aa, Homo sapiens
  DISULFID 46..59: note = Intrachain Disulfide Bond
  DISULFID 54..70: note = Intrachain Disulfide Bond
  DISULFID 72..81: note = Intrachain Disulfide Bond
DLQEADLDLLRVTLSSKPQALATPNKEEHGKRKKKGKGLGKKRDPCLRKYKDFCIHGECKYVKELRAPSCICHPGYHGERCHGLSL

SEQ ID NO: 10 - protein, 42 aa, Danio rerio
  DISULFID 2..15: note = Intrachain Disulfide Bond
  DISULFID 10..26: note = Intrachain Disulfide Bond
  DISULFID 28..37: note = Intrachain Disulfide Bond
ACPKEYKHFCIHGVCRFIEEQNTPSCRCEKGYIGSRCEYLDI

SEQ ID NO: 11 - protein, 50 aa, Myrmecia gulosa
  DISULFID 9..22: note = Intrachain Disulfide Bond
  DISULFID 17..33: note = Intrachain Disulfide Bond
  DISULFID 35..44: note = Intrachain Disulfide Bond
DISDYGDPCSDDLKDYCIHGDCFFLKELNQPACRCYTGYYGSRCEHIDHN

SEQ ID NO: 12 - protein, 50 aa, Homo sapiens
  DISULFID 8..21: note = Intrachain Disulfide Bond
  DISULFID 16..32: note = Intrachain Disulfide Bond
  DISULFID 34..43: note = Intrachain Disulfide Bond
VVSHFNDCPDSHTQFCFHGTCRFLVQEDKPACVCHSGYVGARCEHADLLA

SEQ ID NO: 13 - protein, 53 aa, Homo sapiens
  DISULFID 6..20: note = Intrachain Disulfide Bond
  DISULFID 14..31: note = Intrachain Disulfide Bond
  DISULFID 33..42: note = Intrachain Disulfide Bond
NSDSECPLSHDGYCLHDGVCMYIEALDKYACNCVVGYIGERCQYRDLKWWELR

SEQ ID NO: 14 - protein, 47 aa, Rattus sp.
NSNTGCPPSYDGYCLNGGVCMYVESVDRYVCNCVIGYIGERCQHRDL

SEQ ID NO: 15 - protein, 54 aa, Dicentrarchus labrax
  DISULFID 15..28: note = Intrachain Disulfide Bond
  DISULFID 23..39: note = Intrachain Disulfide Bond
  DISULFID 41..50: note = Intrachain Disulfide Bond
DTIDTGQWNGHFSKCPEELTYYCIHGDCRYIKEQKAPSCRCHHGYIGSRCEYLD

SEQ ID NO: 16 - protein, 51 aa, Cowpox virus
DIPAIRLCGPEGDGYCLHGDCIHARDIDGMYCRCSHGYTGIRCQHVVLVDY

SEQ ID NO: 17 - protein, 80 aa, Homo sapiens
  DISULFID 15..38: note = Intrachain Disulfide Bond
  DISULFID 22..46: note = Intrachain Disulfide Bond
  DISULFID 51..62: note = Intrachain Disulfide Bond
DGNSTRSPETNGLLCGDPEENCAATTTQSKRKGHFSRCPKQYKHYCIKGRCRFVVAEQTPSCVCDEGYIGARCERVDLFY

SEQ ID NO: 18 - protein, 87 aa, Manica rubida
  DISULFID 30..46: note = Intrachain Disulfide Bond
  DISULFID 37..52: note = Intrachain Disulfide Bond
  DISULFID 65..67: note = Intrachain Disulfide Bond
MKNNYNRINTFIVYLMVTFSLISIISITECTPNHDPCPPQYAEALCLNGGTCFSVTIMGSDNYNCICAPGFRGWRCQEKDLDHPVNQ

SEQ ID NO: 19 - protein, 61 aa, Homo sapiens
KEVMPKFCLRPKNPGKCKQALKKFFFNAETSKCEEFIYGGCGGNKNNFDTKKECRLACKKN

SEQ ID NO: 20 - protein, 51 aa, Monkeypox virus; identical to SEQ ID NO: 16
  DISULFID 8..21: note = Intrachain Disulfide Bond
  DISULFID 16..32: note = Intrachain Disulfide Bond
  DISULFID 34..43: note = Intrachain Disulfide Bond

SEQ ID NO: 21 - protein, 44 aa, Rattus sp.
  DISULFID 6..20: note = Intrachain Disulfide Bond
  DISULFID 14..31: note = Intrachain Disulfide Bond
  DISULFID 33..42: note = Intrachain Disulfide Bond
NSITGCPPSYDGYCLNGGVCMYVESVDRYVCNCVIGYIGERCQH

SEQ ID NO: 22 - protein, 58 aa, Homo sapiens
DGAEDPRCLEALKPGNCGEYVVRWYYDKQVNSCARFWFSGGNGSGNRFNSEKECQETC

SEQ ID NO: 23 - protein, 40 aa, unidentified
  DISULFID 3..14: note = Intrachain Disulfide Bond
  DISULFID 8..25: note = Intrachain Disulfide Bond
  DISULFID 27..38: note = Intrachain Disulfide Bond
NLCTPTPCANGGTCILINANTDYFCNCPNTFPLGGKNCDQ

SEQ ID NO: 24 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMENGLKSNVLNDAIKMPINSEEPDTERLLACLLGNGRCSSNRDCCELTPVCKRGSCISSGPGLVGGILGGIL

SEQ ID NO: 25 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMENGLKSNVLNDAIKMSINSEEPDTERLLACLLGNGRCSSNRDCCELTPVCKRGSCISSGPGLVGGILGGIL

SEQ ID NO: 26 - protein, 45 aa, Hadronyche versuta
  DISULFID 4..18: note = Intrachain Disulfide Bond
  DISULFID 11..24: note = Intrachain Disulfide Bond
  DISULFID 17..29: note = Intrachain Disulfide Bond
LLACLFGNGRCSSNRDCCELTPVCKRGSCVSSGPGLVGGILGGIL

SEQ ID NO: 27 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMENGFESNALHDAIKMPINSEKPDTERLLACLFGNGRCSSNKDCCELTPVCKRGSCVSGGPGLVGGILGGIL

SEQ ID NO: 28 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMKNGFESNALHDAIKMPINSEKPDTERLLACLFGNGRCSSNKDCCELTPVCKRGSCVSGGPGLVGGILGGIL

SEQ ID NO: 29 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMENGFESNALHDAIEMPINSEKPDTERLLACLFGNGRCSSNKDCCELTPVCKRGSCVSGGPGLVGGILGGIL

SEQ ID NO: 30 - protein, 79 aa, unidentified
  DISULFID 38..52: note = Intrachain Disulfide Bond
  DISULFID 45..58: note = Intrachain Disulfide Bond
  DISULFID 51..63: note = Intrachain Disulfide Bond
GIKNEDFMENGLESNALHDAIKMPINSEKPDTERLLACLFGNGRCSSNKDCCELTPVCKRGSCVSGGPGLVGGILGGIL

SEQ ID NO: 31 - protein, 80 aa, Hadronyche infensa
  DISULFID 39..53: note = Intrachain Disulfide Bond
  DISULFID 46..59: note = Intrachain Disulfide Bond
  DISULFID 52..64: note = Intrachain Disulfide Bond
GKINEDFMENGLESHALHDEIRKPIDTEKADAERGVLDCVVNTLGCSSDKDCCGMTPSCTLGICAPSVGGIVGGLLGRAL

SEQ ID NO: 32 - protein, 80 aa, unidentified
  DISULFID 39..53: note = Intrachain Disulfide Bond
  DISULFID 46..59: note = Intrachain Disulfide Bond
  DISULFID 52..64: note = Intrachain Disulfide Bond
GKINEDFMENGLESHALHDEIRKPIDTEKADAERGVVGCVLNTLGCSSDKDCCGMTPSCTLGICAPSVGGLVGGLLGRAL

SEQ ID NO: 33 - protein, 45 aa, Atrax sp.
  DISULFID 5..18: note = Intrachain Disulfide Bond
  DISULFID 11..24: note = Intrachain Disulfide Bond
  DISULFID 17..29: note = Intrachain Disulfide Bond
RLLDCVLSRVCSPDANCCGLTPICKMGLCVPKVGGLLGGLLGGIL

SEQ ID NO: 34 - protein, 81 aa, Lychas mucronatus
GYLGLGYGGLYGAYGGLYGAGVPVGRAVAYSSSIRHPGFGGLGVLGGYGGYGYGLGLGAYGYGGYGLGYGLGLGLGHGKIW

SEQ ID NO: 35 - protein, 65 aa, unidentified
NLWQLGNLIGKYTKYLRIKFIDLGNWCGKGGGGTPVDYCDSCCYAHDKCYDANSTPSCFNYLATY

SEQ ID NO: 36 - protein, 50 aa, unidentified
LFSLSGVIRCVTRRSALLMYNNYGCFCGRGGWGAAPVDDTDRCCVAHDNC

SEQ ID NO: 37 - protein, 53 aa, unidentified
DLFSLSGVIRCVTRRSALLMYNNYGCFCGRGGWGAAPVDDTDRCCVAHDNCYA

SEQ ID NO: 38 - protein, 43 aa, unidentified
DLISFAFMMASKVKRCLFDYINYGNYCGVLGAGVPVDSIDKCC

SEQ ID NO: 39 - protein, 45 aa, unidentified
LISFAFMMASKVKRCLFDYINYGNYCGVLGAGVPVDSIDKCCETH

SEQ ID NO: 40 - protein, 35 aa, Bothrops pauloensis
SLWEFAQMILEETKRLPFPYYGAYGCYCGWGGQGQ

SEQ ID NO: 41 - protein, 43 aa, Strongylocentrotus purpuratus
LLEFAIMIKCHTGRWPLDYNGYGCYCGMGGEGRVIDEVDACCK

SEQ ID NO: 42 - protein, 63 aa, unidentified
  DISULFID 12..62: note = Intrachain Disulfide Bond
  DISULFID 21..58: note = Intrachain Disulfide Bond
  DISULFID 37..45: note = Intrachain Disulfide Bond
SKRGAWNNPDRCKGPKIRGPCTKNIRRYHYDSTLDQCVPFSYGGCGGNDNNWWYKKNCEKICL

SEQ ID NO: 43 - protein, 61 aa, unidentified; identical to SEQ ID NO: 19
  DISULFID 8..58: note = Intrachain Disulfide Bond
  DISULFID 17..54: note = Intrachain Disulfide Bond
  DISULFID 33..41: note = Intrachain Disulfide Bond

SEQ ID NO: 44 - protein, 63 aa, unidentified
  DISULFID 4..54: note = Intrachain Disulfide Bond
  DISULFID 13..50: note = Intrachain Disulfide Bond
  DISULFID 29..37: note = Intrachain Disulfide Bond
PKFCLRPKNPGKCKQALKKFFFNAETSKCEEFIYGGCGGNKNNFDTKKECRLACKKNKIMPIL

SEQ ID NO: 45 - protein, 65 aa, unidentified
  DISULFID 7..57: note = Intrachain Disulfide Bond
  DISULFID 16..53: note = Intrachain Disulfide Bond
  DISULFID 32..40: note = Intrachain Disulfide Bond
TKRKHRCKFPLDDGPCRALIPRWWYDYKTNNCTAFYYGGCEGNENNFETKDMCEEKCKRPRPRKP

SEQ ID NO: 46 - protein, 58 aa, unidentified
  DISULFID 8..58: note = Intrachain Disulfide Bond
  DISULFID 17..54: note = Intrachain Disulfide Bond
  DISULFID 33..41: note = Intrachain Disulfide Bond
PESATDICRLQKEEGTCRDFVLKWYYDPKTKSCARFWYGGCGGNENRFNTQKECEKVC

SEQ ID NO: 47 - protein, 57 aa, Lonchura domestica
  DISULFID 7..57: note = Intrachain Disulfide Bond
  DISULFID 16..53: note = Intrachain Disulfide Bond
  DISULFID 32..40: note = Intrachain Disulfide Bond
KPLLDICRLQKDEGTCRSFVLKWYYDPETKSCARFWYGGCGGNENRFNTQKECEKLC

SEQ ID NO: 48 - protein, 8 aa, synthetic construct
DYKDDDDK

SEQ ID NO: 49 - protein, 20 aa, synthetic construct
ASPAAPAPASPAAPAPSAPA

SEQ ID NO: 50 - protein, 40 aa, synthetic construct
ASPAAPAPASPAAPAPSAPAASPAAPAPASPAAPAPSAPA

SEQ ID NO: 51 - protein, 15 aa, synthetic construct
GGGGSGGGGSGGGGS

SEQ ID NO: 52 - protein, 111 aa, synthetic construct
APRPAAPAPASPAAPAPSAPAASPAAPAPASPAAPAPSAPAGGGGSGGGGSGGGGSPSPAAPAPASPAAPAPSAPAASPAAPAPASPAAPAPSAPAGGGGSGGGGSGGGGS

SEQ ID NO: 53 - protein, 221 aa, synthetic construct
APRPAAPAPASPAAPAPSAPAASPAAPAPASPAAPAPSAPAGGGGSGGGGSGGGGSPSPAAPAPASPAAPAPSAPAASPAAPAPASPAAPAPSAPAGGGGSGGGGSGGGGSPSPAAPAPASPAAPAPSAPAASPAAPAPASPAAPAPSAPAGGGGSGGGGSGGGGSPSPAAPAPASPAAPAPSAPAASPAAPAPASPAAPAPSAPAGGGGSGGGGSGGGGS

SEQ ID NO: 54 - protein, 52 aa, unidentified
FCLRPKNPGKCKQALKKFFFNAETSKCEEFIYGGCGGNKNNFDTKKECRLAC

SEQ ID NO: 55 - protein, 53 aa, unidentified
FCLRPKNPGKCKQALKKFFFNAETSKCEEFIYGGCGGNKNNFDTKKECRLACK

SEQ ID NO: 56 - protein, 51 aa, unidentified
CLRPKNPGKCKQALKKFFFNAETSKCEEFIYGGCGGNKNNFDTKKECRLAC

SEQ ID NO: 57 - protein, 58 aa, unidentified
MPKFCLRPKNPGKCKQALKKFFFNAETSKCEEFIYGGCGGNKNNFDTKKECRLACKKN

SEQ ID NO: 58 - protein, 59 aa, unidentified
EVMPKFCLRPKNPGKCKQALKKFFFNAETSKCEEFIYGGCGGNKNNFDTKKECRLACKK

SEQ ID NO: 59 - protein, 57 aa, unidentified
MPKFCLRPKNPGKCKQALKKFFFNAETSKCEEFIYGGCGGNKNNFDTKKECRLACKK

SEQ ID NO: 60 - protein, 57 aa, unidentified
PKFCLRPKNPGKCKQALKKFFFNAETSKCEEFIYGGCGGNKNNFDTKKECRLACKKN

SEQ ID NO: 61 - protein, 55 aa, unidentified
PKFCLRPKNPGKCKQALKKFFFNAETSKCEEFIYGGCGGNKNNFDTKKECRLACK

SEQ ID NO: 62 - protein, 45 aa, unidentified
PGKCKQALKKFFFNAETSKCEEFIYGGCGGNKNNFDTKKECRLAC

SEQ ID NO: 63 - protein, 55 aa, unidentified
MPKFCLRPKNPGKCKQALKKFFFNAETSKCEEFIYGGCGGNKNNFDTKKECRLAC

SEQ ID NO: 64 - protein, 58 aa, unidentified
KEVMPKFCLRPKNPGKCKQALKKFFFNAETSKCEEFIYGGCGGNKNNFDTKKECRLAC

SEQ ID NO: 65 - protein, 57 aa, unidentified
EVMPKFCLRPKNPGKCKQALKKFFFNAETSKCEEFIYGGCGGNKNNFDTKKECRLAC

SEQ ID NO: 66 - protein, 54 aa, unidentified
PKFCLRPKNPGKCKQALKKFFFNAETSKCEEFIYGGCGGNKNNFDTKKECRLAC

SEQ ID NO: 67 - protein, 52 aa, Homo sapiens
VCSEQAETGPCRAMISRWYFDVTEGKCAPFFYGGCGGNRNNFDTEEYCMAVC

SEQ ID NO: 68 - protein, 53 aa, Homo sapiens
VCSQEAMTGPCRAVMPRWYFDLSKGKCVRFIYGGCGGNRNNFESEDYCMAVCK

SEQ ID NO: 69 - protein, 51 aa, Homo sapiens
CSLPALQGPCKAYAPRWAYNSQTGQCQSFVYGGCEGNGNNFESREACEESC

SEQ ID NO: 70 - protein, 58 aa, Homo sapiens
LPNVCAFPMEKGPCQTYMTRWFFNFETGECELFAYGGCGGNSNNFLRKEKCEKFCKFT

SEQ ID NO: 71 - protein, 59 aa, Homo sapiens
KKIPSFCYSPKDEGLCSANVTRYYFNPRYRTCDAFTYTGCGGNDNNFVSREDCKRACAK

SEQ ID NO: 72 - protein, 52 aa, Homo sapiens
FCLVSKVVGRCRASMPRWWYNVTDGSCQLFVYGGCDGNSNNYLTKEECLKKC

SEQ ID NO: 73 - protein, 57 aa, Homo sapiens
MHSFCAFKADDGPCKAIMKRFFFNIFTRQCEEFIYGGCEGNQNRFESLEECKKMCTR

SEQ ID NO: 74 - protein, 57 aa, Homo sapiens
PDFCFLEEDPGICRGYITRYFYNNQTKQCERFKYGGCLGNMNNFETLEECKNICEDG

SEQ ID NO: 75 - protein, 57 aa, Homo sapiens
PSWCLTPADRGLCRANENRFYYNSVIGKCRPFKYSGCGGNENNFTSKQECLRACKKG

SEQ ID NO: 76 - protein, 52 aa, Homo sapiens
SCQLGYSAGPCMGMTSRYFYNGTSMACETFQYGGCMGNGNNFVTEKECLQTC

SEQ ID NO: 77 - protein, 55 aa, Homo sapiens
QDVCEMPKETGPCLAYFLHWWYDKKDNTCSMFVYGGCQGNNNNFQSKANCLNTCK

SEQ ID NO: 78 - protein, 45 aa, Homo sapiens
QGPCRGWEPRWAYSPLLQQCHPFVYGGCEGNGNNFHSRESCEDAC

SEQ ID NO: 79 - protein, 55 aa, Homo sapiens
FQEPCMLPVRHGNCNHEAQRWHFDFKNYRCTPFKYRGCEGNANNFLNEDACRTAC

SEQ ID NO: 80 - protein, 58 aa, Homo sapiens
DGAEDPRCLEALKPGNCGEYVVRWYYDKQVNSCARFWFSGCNGSGNRFNSEKECQETC

SEQ ID NO: 81 - protein, 53 aa, Homo sapiens
HCVDLPDTGLCKESIPRWYYNPFSEHCARFTYGGCYGNKNNFEEEQQCLESCR

SEQ ID NO: 82 - protein, 53 aa, Homo sapiens
PCKLDMNFGSCYEVHFRYFYNRTSKRCETFVFSGCNGNLNNFKLKIEREVACV

SEQ ID NO: 83 - protein, 62 aa, Homo sapiens
VGQPSHAYPVRCLLPSAHGSCADWAARWYFVASVGQCNRFWYGGCHGNANNFASEQECMSSC

SEQ ID NO: 84 - protein, 54 aa, Homo sapiens
TDICKLPKDEGTCRDFILKWYYDPNTKSCARFWYGGCGGNENKFGSQKECEKVC